SEQUENCE LISTING (144 sequences)

SEQ ID NO: 1 - DNA, 5777 bp, Homo sapiens
  misc_feature 1..5777: note = VEGFR-1
  source 1..5777: mol_type = genomic DNA
  CDS 250..4266
gcggacactcctctcggctcctccccggcagcggcggcggctcggagcgggctccggggctcgggtgcagcggccagcgggcctggcggcgaggattacccggggaagtggttgtctcctggctggagccgcgagacgggcgctcagggcgcggggccggcggcggcgaacgagaggacggactctggcggccgggtcgttggccgggggagcgcgggcaccgggcgagcaggccgcgtcgcgctcaccatggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagagatcaggaagcaccatacctcctgcgaaacctcagtgatcacacagtggccatcagcagttccaccactttagactgtcatgctaatggtgtccccgagcctcagatcacttggtttaaaaacaaccacaaaatacaacaagagcctggaattattttaggaccaggaagcagcacgctgtttattgaaagagtcacagaagaggatgaaggtgtctatcactgcaaagccaccaaccagaagggctctgtggaaagttcagcatacctcactgttcaaggaacctcggacaagtctaatctggagctgatcactctaacatgcacctgtgtggctgcgactctcttctggctcctattaaccctccttatccgaaaaatgaaaaggtcttcttctgaaataaagactgactacctatcaattataatggacccagatgaagttcctttggatgagcagtgtgagcggctcccttatgatgccagcaagtgggagtttgcccgggagagacttaaactgggcaaatcacttggaagaggggcttttggaaaagtggttcaagcatcagcatttggcattaagaaatcacctacgtgccggactgtggctgtgaaaatgctgaaagagggggccacggccagcgagtacaaagctctgatgactgagctaaaaatcttgacccacattggccaccatctgaacgtggttaacctgctgggagcctgcaccaagcaaggagggcctctgatggtgattgttgaatactgcaaatatggaaatctctccaactacctcaagagcaaacgtgacttattttttctcaacaaggatgcagcactacacatggagcctaagaaagaaaaaatggagccaggcctggaacaaggcaagaaaccaagactagatagcgtcaccagcagcgaaagctttgcgagctccggctttcaggaagataaaagtctgagtgatgttgaggaagaggaggattctgacggtttctacaaggagcccatcactatggaagatctgatttcttacagttttcaagtggccagaggcatggagttcctgtcttccagaaagtgcattcatcgggacctggcagcgagaaacattcttttatctgagaacaacgtggtgaagatttgtgattttggccttgcccgggatatttataagaaccccgattatgtgagaaaaggagatactcgacttcctctgaaatggatggctcccgaatctatctttgacaaaatctacagcaccaagagcgacgtgtggtcttacggagtattgctgtgggaaatcttctccttaggtgggtctccatacccaggagtacaaatggatgaggacttttgcagtcgcctgagggaaggcatgaggatgagagctcctgagtactctactcctgaaatctatcagatcatgctggactgctggcacagagacccaaaagaaaggccaagatttgcagaacttgtggaaaaactaggtgatttgcttcaagcaaatgtacaacaggatggtaaagactacatcccaatcaatgccatactgacaggaaatagtgggtttacatactcaactcctgccttctctgaggacttcttcaaggaaagtatttcagctccgaagtttaattcaggaagctctgatgatgtcagatatgtaaatgctttcaagttcatgagcctggaaagaatcaaaacctttgaagaacttttaccgaatgccacctccatgtttgatgactaccagggcgacagcagcactctgttggcctctcccatgctgaagcgcttcacctggactgacagcaaacccaaggcctcgctcaagattgacttgagagtaaccagtaaaagtaaggagtcggggctgtctgatgtcagcaggcccagtttctgccattccagctgtgggcacgtcagcgaaggcaagcgcaggttcacctacgaccacgctgagctggaaaggaaaatcgcgtgctgctccccgcccccagactacaactcggtggtcctgtactccaccccacccatctagagtttgacacgaagccttatttctagaagcacatgtgtatttatacccccaggaaactagcttttgccagtattatgcatatataagtttacacctttatctttccatgggagccagctgctttttgtgatttttttaatagtgctttttttttttgactaacaagaatgtaactccagatagagaaatagtgacaagtgaagaacactactgctaaatcctcatgttactcagtgttagagaaatccttcctaaacccaatgacttccctgctccaacccccgccacctcagggcacgcaggaccagtttgattgaggagctgcactgatcacccaatgcatcacgtaccccactgggccagccctgcagcccaaaacccagggcaacaagcccgttagccccaggggatcactggctggcctgagcaacatctcgggagtcctctagcaggcctaagacatgtgaggaggaaaaggaaaaaaagcaaaaagcaagggagaaaagagaaaccgggagaaggcatgagaaagaatttgagacgcaccatgtgggcacggagggggacggggctcagcaatgccatttcagtggcttcccagctctgacccttctacatttgagggcccagccaggagcagatggacagcgatgaggggacattttctggattctgggaggcaagaaaaggacaaatatcttttttggaactaaagcaaattttagacctttacctatggaagtggttctatgtccattctcattcgtggcatgttttgatttgtagcactgagggtggcactcaactctgagcccatacttttggctcctctagtaagatgcactgaaaacttagccagagttaggttgtctccaggccatgatggccttacactgaaaatgtcacattctattttgggtattaatatatagtccagacacttaactcaatttcttggtattattctgttttgcacagttagttgtgaaagaaagctgagaagaatgaaaatgcagtcctgaggagagttttctccatatcaaaacgagggctgatggaggaaaaaggtcaataaggtcaagggaagaccccgtctctataccaaccaaaccaattcaccaacacagttgggacccaaaacacaggaagtcagtcacgtttccttttcatttaatggggattccactatctcacactaatctgaaaggatgtggaagagcattagctggcgcatattaagcactttaagctccttgagtaaaaaggtggtatgtaatttatgcaaggtatttctccagttgggactcaggatattagttaatgagccatcactagaagaaaagcccattttcaactgctttgaaacttgcctggggtctgagcatgatgggaatagggagacagggtaggaaagggcgcctactcttcagggtctaaagatcaagtgggccttggatcgctaagctggctctgtttgatgctatttatgcaagttagggtctatgtattta

SEQ ID NO: 2 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLLIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 3 - DNA, 2292 bp, Homo sapiens
  source 1..2292: mol_type = genomic DNA
  CDS 1..2292
atggagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaa

SEQ ID NO: 4 - protein, 764 aa, Homo sapiens
MESKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLE

SEQ ID NO: 5 - DNA, 4195 bp, Homo sapiens
  source 1..4195: mol_type = genomic DNA
  CDS 20..3913
ccacgcgcagcggccggagatgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctgaactgcaccgtgtgggctgagtttaactcaggtgtcacctttgactgggactacccagggaagcaggcagagcggggtaagtgggtgcccgagcgacgctcccagcagacccacacagaactctccagcatcctgaccatccacaacgtcagccagcacgacctgggctcgtatgtgtgcaaggccaacaacggcatccagcgatttcgggagagcaccgaggtcattgtgcatgaaaatcccttcatcagcgtcgagtggctcaaaggacccatcctggaggccacggcaggagacgagctggtgaagctgcccgtgaagctggcagcgtaccccccgcccgagttccagtggtacaaggatggaaaggcactgtccgggcgccacagtccacatgccctggtgctcaaggaggtgacagaggccagcacaggcacctacaccctcgccctgtggaactccgctgctggcctgaggcgcaacatcagcctggagctggtggtgaatgtgcccccccagatacatgagaaggaggcctcctcccccagcatctactcgcgtcacagccgccaggccctcacctgcacggcctacggggtgcccctgcctctcagcatccagtggcactggcggccctggacaccctgcaagatgtttgcccagcgtagtctccggcggcggcagcagcaagacctcatgccacagtgccgtgactggagggcggtgaccacgcaggatgccgtgaaccccatcgagagcctggacacctggaccgagtttgtggagggaaagaataagactgtgagcaagctggtgatccagaatgccaacgtgtctgccatgtacaagtgtgtggtctccaacaaggtgggccaggatgagcggctcatctacttctatgtgaccaccatccccgacggcttcaccatcgaatccaagccatccgaggagctactagagggccagccggtgctcctgagctgccaagccgacagctacaagtacgagcatctgcgctggtaccgcctcaacctgtccacgctgcacgatgcgcacgggaacccgcttctgctcgactgcaagaacgtgcatctgttcgccacccctctggccgccagcctggaggaggtggcacctggggcgcgccacgccacgctcagcctgagtatcccccgcgtcgcgcccgagcacgagggccactatgtgtgcgaagtgcaagaccggcgcagccatgacaagcactgccacaagaagtacctgtcggtgcaggccctggaagcccctcggctcacgcagaacttgaccgacctcctggtgaacgtgagcgactcgctggagatgcagtgcttggtggccggagcgcacgcgcccagcatcgtgtggtacaaagacgagaggctgctggaggaaaagtctggagtcgacttggcggactccaaccagaagctgagcatccagcgcgtgcgcgaggaggatgcgggacgctatctgtgcagcgtgtgcaacgccaagggctgcgtcaactcctccgccagcgtggccgtggaaggctccgaggataagggcagcatggagatcgtgatccttgtcggtaccggcgtcatcgctgtcttcttctgggtcctcctcctcctcatcttctgtaacatgaggaggccggcccacgcagacatcaagacgggctacctgtccatcatcatggaccccggggaggtgcctctggaggagcaatgcgaatacctgtcctacgatgccagccagtgggaattcccccgagagcggctgcacctggggagagtgctcggctacggcgccttcgggaaggtggtggaagcctccgctttcggcatccacaagggcagcagctgtgacaccgtggccgtgaaaatgctgaaagagggcgccacggccagcgagcaccgcgcgctgatgtcggagctcaagatcctcattcacatcggcaaccacctcaacgtggtcaacctcctcggggcgtgcaccaagccgcagggccccctcatggtgatcgtggagttctgcaagtacggcaacctctccaacttcctgcgcgccaagcgggacgccttcagcccctgcgcggagaagtctcccgagcagcgcggacgcttccgcgccatggtggagctcgccaggctggatcggaggcggccggggagcagcgacagggtcctcttcgcgcggttctcgaagaccgagggcggagcgaggcgggcttctccagaccaagaagctgaggacctgtggctgagcccgctgaccatggaagatcttgtctgctacagcttccaggtggccagagggatggagttcctggcttcccgaaagtgcatccacagagacctggctgctcggaacattctgctgtcggaaagcgacgtggtgaagatctgtgactttggccttgcccgggacatctacaaagaccctgactacgtccgcaagggcagtgcccggctgcccctgaagtggatggcccctgaaagcatcttcgacaaggtgtacaccacgcagagtgacgtgtggtcctttggggtgcttctctgggagatcttctctctgggggcctccccgtaccctggggtgcagatcaatgaggagttctgccagcggctgagagacggcacaaggatgagggccccggagctggccactcccgccatacgccgcatcatgctgaactgctggtccggagaccccaaggcgagacctgcattctcggagctggtggagatcctgggggacctgctccagggcaggggcctgcaagaggaagaggaggtctgcatggccccgcgcagctctcagagctcagaagagggcagcttctcgcaggtgtccaccatggccctacacatcgcccaggctgacgctgaggacagcccgccaagcctgcagcgccacagcctggccgccaggtattacaactgggtgtcctttcccgggtgcctggccagaggggctgagacccgtggttcctccaggatgaagacatttgaggaattccccatgaccccaacgacctacaaaggctctgtggacaaccagacagacagtgggatggtgctggcctcggaggagtttgagcagatagagagcaggcatagacaagaaagcggcttcaggtagctgaagcagagagagagaaggcagcatacgtcagcattttcttctctgcacttataagaaagatcaaagactttaagactttcgctatttcttctactgctatctactacaaacttcaaagaggaaccaggaggacaagaggagcatgaaagtggacaaggagtgtgaccactgaagcaccacagggaaggggttaggcctccggatgactgcgggcaggcctggataatatccagcctcccacaagaagctggtggagcagagtgttccctgactcct

SEQ ID NO: 6 - protein, 1298 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFR

SEQ ID NO: 7 - DNA, 1713 bp, synthetic construct
  misc_feature 1..1713: note = Synthetic Construct
  misc_feature 1..1713: note = R-2 A
  source 1..1713: mol_type = other DNA
  CDS 1..1713
atgccgctgctgctactgctgcccctgctgtgggcaggggccctggctatggataagcttgctagcggatccttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaagatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 8 - protein, 570 aa, synthetic construct
  REGION 1..570: note = Synthetic Construct
MPLLLLLPLLWAGALAMDKLASGSLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 9 - DNA, 1416 bp, synthetic construct
  misc_feature 1..1416: note = Synthetic Construct
  misc_feature 1..1416: note = R-2 B
  source 1..1416: mol_type = other DNA
  CDS 1..1416
atgccgctgctgctactgctgcccctgctgtgggcaggggccctggctatggataagcttgctagcggtaccctcgaggatggccgcggatccttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtaggggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 10 - protein, 471 aa, synthetic construct
  REGION 1..471: note = Synthetic Construct
MPLLLLLPLLWAGALAMDKLASGTLEDGRGSLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 11 - DNA, 1434 bp, synthetic construct
  misc_feature 1..1434: note = Synthetic Construct
  misc_feature 1..1434: note = R-2 C
  source 1..1434: mol_type = other DNA
  CDS 1..1434
atgccgctgctgctactgctgcccctgctgtgggcaggggccctggctatggataagcttgctagcggtaccctcgaggatggccgcggatccttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 12 - protein, 477 aa, synthetic construct
  REGION 1..477: note = Synthetic Construct
MPLLLLLPLLWAGALAMDKLASGTLEDGRGSLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 13 - DNA, 1452 bp, synthetic construct
  misc_feature 1..1452: note = Synthetic Construct
  misc_feature 1..1452: note = R-2 D
  source 1..1452: mol_type = other DNA
  CDS 1..1452
atgccgctgctgctactgctgcccctgctgtgggcaggggccctggctatggataagcttgctagcggtaccctcgaggatggccgcggatccttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatgatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - protein, 483 aa, synthetic construct
  REGION 1..483: note = Synthetic Construct
MPLLLLLPLLWAGALAMDKLASGTLEDGRGSLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 15 - DNA, 1479 bp, synthetic construct
  misc_feature 1..1479: note = Synthetic Construct
  misc_feature 1..1479: note = R-2 E
  source 1..1479: mol_type = other DNA
  CDS 1..1479
atgccgctgctgctactgctgcccctgctgtgggcaggggccctggctatggataagcttgctagcggtaccctcgaggatggccgcggatccttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaaggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 16 - protein, 492 aa, synthetic construct
  REGION 1..492: note = Synthetic Construct
MPLLLLLPLLWAGALAMDKLASGTLEDGRGSLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 17 - DNA, 1113 bp, synthetic construct
  misc_feature 1..1113: note = Synthetic Construct
  misc_feature 1..1113: note = R-2 F
  source 1..1113: mol_type = other DNA
  CDS 1..1113
atgccgctgctgctactgctgcccctgctgtgggcaggggccctggctatggataagcttgctagcggtaccctcgaggatggccgcggatccttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 18 - protein, 370 aa, synthetic construct
  REGION 1..370: note = Synthetic Construct
MPLLLLLPLLWAGALAMDKLASGTLEDGRGSLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 19 - DNA, 1425 bp, synthetic construct
  misc_feature 1..1425: note = Synthetic Construct
  misc_feature 1..1425: note = R-2 G
  source 1..1425: mol_type = other DNA
  CDS 1..1425
atgccgctgctgctactgctgcccctgctgtgggcaggggccctggctatggataagcttgctagcgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaagatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 20 - protein, 474 aa, synthetic construct
  REGION 1..474: note = Synthetic Construct
MPLLLLLPLLWAGALAMDKLASVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 21 - DNA, 1107 bp, synthetic construct
  misc_feature 1..1107: note = Synthetic Construct
  misc_feature 1..1107: note = R-2 H
  source 1..1107: mol_type = other DNA
  CDS 1..1107
atgccgctgctgctactgctgcccctgctgtgggcaggggccctggctatggataagcttgctagcgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtaggggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 22 - protein, 368 aa, synthetic construct
  REGION 1..368: note = Synthetic Construct
MPLLLLLPLLWAGALAMDKLASVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 23 - DNA, 1125 bp, synthetic construct
  misc_feature 1..1125: note = Synthetic Construct
  misc_feature 1..1125: note = R-2 I
  source 1..1125: mol_type = other DNA
  CDS 1..1125
atgccgctgctgctactgctgcccctgctgtgggcaggggccctggctatggataagcttgctagcgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 24 - protein, 374 aa, synthetic construct
  REGION 1..374: note = Synthetic Construct
MPLLLLLPLLWAGALAMDKLASVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 25 - DNA, 1143 bp, synthetic construct
  misc_feature 1..1143: note = Synthetic Construct
  misc_feature 1..1143: note = R-2 J
  source 1..1143: mol_type = other DNA
  CDS 1..1143
atgccgctgctgctactgctgcccctgctgtgggcaggggccctggctatggataagcttgctagcgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatgatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 26 - protein, 380 aa, synthetic construct
  REGION 1..380: note = Synthetic Construct
MPLLLLLPLLWAGALAMDKLASVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 27 - DNA, 1170 bp, synthetic construct
  misc_feature 1..1170: note = Synthetic Construct
  misc_feature 1..1170: note = R-2 K
  source 1..1170: mol_type = other DNA
  CDS 1..1170
atgccgctgctgctactgctgcccctgctgtgggcaggggccctggctatggataagcttgctagcgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaaggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 28 - protein, 389 aa, synthetic construct
  REGION 1..389: note = Synthetic Construct
MPLLLLLPLLWAGALAMDKLASVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 29 - DNA, 1116 bp, synthetic construct
  misc_feature 1..1116: note = Synthetic Construct
  misc_feature 1..1116: note = R-2 L
  source 1..1116: mol_type = other DNA
  CDS 1..1116
atgccgctgctgctactgctgcccctgctgtgggcaggggccctggctatggataagcttgctagctataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaagatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 30 - protein, 371 aa, synthetic construct
  REGION 1..371: note = Synthetic Construct
MPLLLLLPLLWAGALAMDKLASYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 31 - DNA, 1368 bp, synthetic construct
  misc_feature 1..1368: note = Synthetic Construct
  misc_feature 1..1368: note = R-3 A
  source 1..1368: mol_type = other DNA
  CDS 1..1368
atgccgctgctgctactgctgcccctgctgtgggcaggggccctggctatggataagcttccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctgaactgcaccgtgtgggctgagtttaactcaggtgtcacctttgactgggactacccagggaagcaggcagagcggggtaagtgggtgcccgagcgacgctcccagcagacccacacagaactctccagcatcctgaccatccacaacgtcagccagcacgacctgggctcgtatgtgtgcaaggccaacaacggcatccagcgatttcgggagagcaccgaggtcattgtgcatgaggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 32 - protein, 455 aa, synthetic construct
  REGION 1..455: note = Synthetic Construct
MPLLLLLPLLWAGALAMDKLPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHEDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 33 - DNA, 1059 bp, synthetic construct
  misc_feature 1..1059: note = Synthetic Construct
  misc_feature 1..1059: note = R-3 B
  source 1..1059: mol_type = other DNA
  CDS 1..1059
atgccgctgctgctactgctgcccctgctgtgggcaggggccctggctatggataagcttccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 34 - protein, 352 aa, synthetic construct
  REGION 1..352: note = Synthetic Construct
MPLLLLLPLLWAGALAMDKLPFINKPDTLLVNRKDAMWVPCLVSIPGLNVTLRSQSSVLWPDGQEVVWDDRRGMLVSTPLLHDALYLQCETTWGDQDFLSNPFLVHITGDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 35 - DNA, 1422 bp, synthetic construct
  misc_feature 1..1422: note = Synthetic Construct
  misc_feature 1..1422: note = R-3 C
  source 1..1422: mol_type = other DNA
  CDS 1..1422
atgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctcgcggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 36 - protein, 473 aa, synthetic construct
  REGION 1..473: note = Synthetic Construct
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELADPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 37 - DNA, 1410 bp, synthetic construct
  misc_feature 1..1410: note = Synthetic Construct
  misc_feature 1..1410: note = R-3 D
  source 1..1410: mol_type = other DNA
  CDS 1..1410
atgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 38 - protein, 469 aa, synthetic construct
  REGION 1..469: note = Synthetic Construct
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 39 - DNA, 1404 bp, synthetic construct
  misc_feature 1..1404: note = Synthetic Construct
  misc_feature 1..1404: note = R-3 E
  source 1..1404: mol_type = other DNA
  CDS 1..1404
atgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacgcggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 40 - protein, 467 aa, synthetic construct
  REGION 1..467: note = Synthetic Construct
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHADPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 41 - DNA, 1395 bp, synthetic construct
  misc_feature 1..1395: note = Synthetic Construct
  misc_feature 1..1395: note = R-3 F
  source 1..1395: mol_type = other DNA
  CDS 1..1395
atgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcgcggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 42 - protein, 464 aa, synthetic construct
  REGION 1..464: note = Synthetic Construct
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFADPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 43 - DNA, 1719 bp, synthetic construct
  misc_feature 1..1719: note = Synthetic Construct
  misc_feature 1..1719: note = R-3 G
  source 1..1719: mol_type = other DNA
  CDS 1..1719
atgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctgaactgcaccgtgtgggctgagtttaactcaggtgtcacctttgactgggactacccagggaagcaggcagagcggggtaagtgggtgcccgagcgacgctcccagcagacccacacagaactctccagcatcctgaccatccacaacgtcagccagcacgacctgggctcgtatgtgtgcaaggccaacaacggcatccagcgatttcgggagagcaccgaggtcattgtgcatgaggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 44 - protein, 572 aa, synthetic construct [sequence omitted]

SEQ ID NO: 45 - DNA, 1131 bp, synthetic construct
  misc_feature 1..1131: note = Synthetic Construct
  misc_feature 1..1131: note = R-3 H
  source 1..1131: mol_type = other DNA
  CDS 1..1131
atgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 46 - protein, 376 aa, synthetic construct
  REGION 1..376: note = Synthetic Construct
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 47 - DNA, 1443 bp, synthetic construct
  misc_feature 1..1443: note = Synthetic Construct
  misc_feature 1..1443: note = R-3 I
  source 1..1443: mol_type = other DNA
  CDS 1..1443
atgcagcggggcgccgcgctgtgcctgcgactgtggctctgcctgggactcctggacggcctggtgagtggctactccatgacccccccgaccttgaacatcacggaggagtcacacgtcagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctgaactgcaccgtgtgggctgagtttaactcaggtgtcacctttgactgggactacccagggaagcaggcagagcggggtaagtgggtgcccgagcgacgctcccagcagacccacacagaactctccagcatcctgaccatccacaacgtcagccagcacgacctgggctcgtatgtgtgcaaggccaacaacggcatccagcgatttcgggagagcaccgaggtcattgtgcatgaggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 48 - protein, 480 aa, synthetic construct
  REGION 1..480: note = Synthetic Construct
MQRGAALCLRLWLCLGLLDGLVSGYSMTPPTLNITEESHVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHEDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 49 - DNA, 1197 bp, synthetic construct
  misc_feature 1..1197: note = Synthetic Construct
  misc_feature 1..1197: note = R-3 J
  source 1..1197: mol_type = other DNA
  CDS 1..1197
atgcagcggggcgccgcgctgtgcctgcgactgtggctctgcctgggactcctggacggcctggtgagtggctactccatgacccccccgaccttgaacatcacggaggagtcacacgtcagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaaggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 50 - protein, 398 aa, synthetic construct
  REGION 1..398: note = Synthetic Construct
MQRGAALCLRLWLCLGLLDGLVSGYSMTPPTLNITEESHVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 51 - DNA, 1146 bp, synthetic construct
  misc_feature 1..1146: note = Synthetic Construct
  misc_feature 1..1146: note = R-3 K
  source 1..1146: mol_type = other DNA
  CDS 1..1146
atgcagcggggcgccgcgctgtgcctgcgactgtggctctgcctgggactcctggacggcctggtgagtggctactccatgacccccccgaccttgaacatcacggaggagtcacacgtcagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctcgcggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 52 - protein, 381 aa, synthetic construct
  REGION 1..381: note = Synthetic Construct
MQRGAALCLRLWLCLGLLDGLVSGYSMTPPTLNITEESHVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELADPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 53 - DNA, 1128 bp, synthetic construct
  misc_feature 1..1128: note = Synthetic Construct
  misc_feature 1..1128: note = R-3 L
  source 1..1128: mol_type = other DNA
  CDS 1..1128
atgcagcggggcgccgcgctgtgcctgcgactgtggctctgcctgggactcctggacggcctggtgagtggctactccatgacccccccgaccttgaacatcacggaggagtcacacgtcagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacgcggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 54 - protein, 375 aa, synthetic construct
  REGION 1..375: note = Synthetic Construct
MQRGAALCLRLWLCLGLLDGLVSGYSMTPPTLNITEESHVRDFEQPFINKPDTLLVNRKDAMWVPCLVSIPGLNVTLRSQSSVLWPDGQEVVWDDRRGMLVSTPLLHDALYLQCETTWGDQDFLSNPFLVHADPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 55 - DNA, 1119 bp, synthetic construct
  misc_feature 1..1119: note = Synthetic Construct
  misc_feature 1..1119: note = R-3 M
  source 1..1119: mol_type = other DNA
  CDS 1..1119
atgcagcggggcgccgcgctgtgcctgcgactgtggctctgcctgggactcctggacggcctggtgagtggctactccatgacccccccgaccttgaacatcacggaggagtcacacgtcagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcgcggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 56 - protein, 372 aa, synthetic construct
  REGION 1..372: note = Synthetic Construct
MQRGAALCLRLWLCLGLLDGLVSGYSMTPPTLNITEESHVRDFEQPFINKPDTLLVNRKDAMWVPCLVSIPGLNVTLRSQSSVLWPDGQEVVWDDRRGMLVSTPLLHDALYLQCETTWGDQDFLSNPFADPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 57 - DNA, 1161 bp, synthetic construct
  misc_feature 1..1161: note = Synthetic Construct
  misc_feature 1..1161: note = R-3 N
  source 1..1161: mol_type = other DNA
  CDS 1..1161
atgcagcggggcgccgcgctgtgcctgcgactgtggctctgcctgggactcctggacggcctggtgagtggctactccatgacccccccgaccttgaacatcacggaggagtcacacgtcaacgagctctatgacatccagctgttgcccaggaagtcgctggagctgctggtaggggagaagctggtcctgaactgcaccgtgtgggctgagtttaactcaggtgtcacctttgactgggactacccagggaagcaggcagagcggggtaagtgggtgcccgagcgacgctcccagcagacccacacagaactctccagcatcctgaccatccacaacgtcagccagcacgacctgggctcgtatgtgtgcaaggccaacaacggcatccagcgatttcgggagagcaccgaggtcattgtgcatgaggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 58 - protein, 386 aa, synthetic construct
  REGION 1..386: note = Synthetic Construct
MQRGAALCLRLWLCLGLLDGLVSGYSMTPPTLNITEESHVNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHEDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 59 - DNA, 27 bp, synthetic construct
  misc_feature 1..27: note = Synthetic primer
  misc_feature 1..27: note = VEGFR-2 D1 reverse primer
  source 1..27: mol_type = other DNA
gctggatcttgaacatagacataaatg

SEQ ID NO: 60 - DNA, 27 bp, synthetic construct
  misc_feature 1..27: note = Synthetic primer
  misc_feature 1..27: note = VEGFR-2 D1-2 reverse primer #1
  source 1..27: mol_type = other DNA
ctaggatcccctacaacgacaactatg

SEQ ID NO: 61 - DNA, 27 bp, synthetic construct
  misc_feature 1..27: note = Synthetic primer
  misc_feature 1..27: note = VEGFR-2 D1-2 reverse primer #2
  source 1..27: mol_type = other DNA
ctaggatccacatcataaatcctatac

SEQ ID NO: 62 - DNA, 36 bp, synthetic construct
  misc_feature 1..36: note = Synthetic primer
  misc_feature 1..36: note = VEGFR-2 D1-2 reverse primer #3
  source 1..36: mol_type = other DNA
gcatggtctcggatcatgagaagacggactcagaac

SEQ ID NO: 63 - DNA, 26 bp, synthetic construct
  misc_feature 1..26: note = Synthetic primer
  misc_feature 1..26: note = VEGFR-2 D1-2 reverse primer #4
  source 1..26: mol_type = other DNA
ctaggatccttttctccaacagatag

SEQ ID NO: 64 - DNA, 29 bp, synthetic construct
  misc_feature 1..29: note = Synthetic primer
  misc_feature 1..29: note = VEGFR-2 D2 forward primer
  source 1..29: mol_type = other DNA
agcgctagcgttcaagattacagatctcc

SEQ ID NO: 65 - DNA, 21 bp, synthetic construct
  misc_feature 1..21: note = Synthetic primer
  misc_feature 1..21: note = VEGFR-2 D2-3 reverse primer
  source 1..21: mol_type = other DNA
atgtgtgaggttttgcacaag

SEQ ID NO: 66 - DNA, 27 bp, synthetic construct
  misc_feature 1..27: note = Synthetic primer
  misc_feature 1..27: note = VEGFR-2 D2 reverse primer #1
  source 1..27: mol_type = other DNA
ctaggatcccctacaacgacaactatg

SEQ ID NO: 67 - DNA, 27 bp, synthetic construct
  misc_feature 1..27: note = Synthetic primer
  misc_feature 1..27: note = VEGFR-2 D2 reverse primer #2
  source 1..27: mol_type = other DNA
ctaggatccacatcataaatcctatac

SEQ ID NO: 68 - DNA, 36 bp, synthetic construct; identical to SEQ ID NO: 62
  misc_feature 1..36: note = Synthetic primer
  misc_feature 1..36: note = VEGFR-2 D2 reverse primer #3
  source 1..36: mol_type = other DNA

SEQ ID NO: 69 - DNA, 26 bp, synthetic construct
  misc_feature 1..26: note = Synthetic primer
  misc_feature 1..26: note = VEGFR-2 D2 reverse primer #4
  source 1..26: mol_type = other DNA
ctaggatccttttctccaacagatag

SEQ ID NO: 70 - DNA, 27 bp, synthetic construct
  misc_feature 1..27: note = Synthetic primer
  misc_feature 1..27: note = VEGFR-2 D3 forward primer
  source 1..27: mol_type = other DNA
agcgctagctataggatttatgatgtg

SEQ ID NO: 71 - DNA, 21 bp, synthetic construct
  misc_feature 1..21: note = Synthetic primer
  misc_feature 1..21: note = VEGFR-2 D3 reverse primer
  source 1..21: mol_type = other DNA
atgtgtgaggttttgcacaag

SEQ ID NO: 72 - DNA, 30 bp, synthetic construct
  misc_feature 1..30: note = Synthetic primer
  misc_feature 1..30: note = VEGFR-2 D1-3 primer 1
  source 1..30: mol_type = other DNA
gcggatccttgcctagtgtttctcttgatc

SEQ ID NO: 73 - DNA, 40 bp, synthetic construct
  misc_feature 1..40: note = Synthetic primer
  misc_feature 1..40: note = VEGFR-2 D1-3 primer 2
  source 1..40: mol_type = other DNA
ccagtcacctgctccggatcttcatggaccctgacaaatg

SEQ ID NO: 74 - DNA, 25 bp, synthetic construct
  misc_feature 1..25: note = Synthetic primer
  misc_feature 1..25: note = VEGFR-3 D1-2 reverse primer 1
  source 1..25: mol_type = other DNA
tcaggatccgcgagctcgttgcctg

SEQ ID NO: 75 - DNA, 27 bp, synthetic construct
  misc_feature 1..27: note = Synthetic primer
  misc_feature 1..27: note = VEGFR-3 D1-2 reverse primer 2
  source 1..27: mol_type = other DNA
tacaggatcccctgtgatgtgcaccag

SEQ ID NO: 76 - DNA, 25 bp, synthetic construct
  misc_feature 1..25: note = Synthetic primer
  misc_feature 1..25: note = VEGFR-3 D1-2 reverse primer 3
  source 1..25: mol_type = other DNA
tcaggatccgcgtgcaccaggaagg

SEQ ID NO: 77 - DNA, 26 bp, synthetic construct
  misc_feature 1..26: note = Synthetic primer
  misc_feature 1..26: note = VEGFR-3 D1-2 reverse primer 4
  source 1..26: mol_type = other DNA
tcaggatccgcgaaggggttggaaag

SEQ ID NO: 78 - DNA, 60 bp, synthetic construct
  misc_feature 1..60: note = Synthetic primer
  misc_feature 1..60: note = VEGFR-3 Delta D1 primer 1
  source 1..60: mol_type = other DNA
ccttgaacatcacggaggagtcacacgtcagagactttgagcagccattcatcaacaagc

SEQ ID NO: 79 - DNA, 42 bp, synthetic construct
  misc_feature 1..42: note = Synthetic primer
  misc_feature 1..42: note = VEGFR-3 Delta D1 primer 2
  source 1..42: mol_type = other DNA
agctgctggtaggggagaaggatcctgaactgcaccgtgtgg

SEQ ID NO: 80 - DNA, 990 bp, Homo sapiens
  misc_feature 1..990: note = VEGF-A
  source 1..990: mol_type = genomic DNA
  CDS 57..629
cagtgtgctggcggcccggcgcgagccggcccggccccggtcgggcctccgaaaccatgaactttctgctgtcttgggtgcattggagcctcgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacaccgcggccgccagcacactg

SEQ ID NO: 81 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 82 - DNA, 1997 bp, Homo sapiens
  misc_feature 1..1997: note = VEGF-C
  source 1..1997: mol_type = genomic DNA
  CDS 352..1608
cccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 83 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 84 - DNA, 1645 bp, Homo sapiens
  misc_feature 1..1645: note = PIGF
  source 1..1645: mol_type = genomic DNA
  CDS 322..768
gggattcgggccgcccagctacgggaggacctggagtggcactgggcgcccgacggaccatccccgggacccgcctgcccctcggcgccccgccccgccgggccgctccccgtcgggttccccagccacagccttacctacgggctcctgactccgcaaggcttccagaagatgctcgaaccaccggccggggcctcggggcagcagtgagggaggcgtccagccccccactcagctcttctcctcctgtgccaggggctccccgggggatgagcatggtggttttccctcggagccccctggctcgggacgtctgagaagatgccggtcatgaggctgttcccttgcttcctgcagctcctggccgggctggcgctgcctgctgtgcccccccagcagtgggccttgtctgctgggaacggctcgtcagaggtggaagtggtacccttccaggaagtgtggggccgcagctactgccgggcgctggagaggctggtggacgtcgtgtccgagtaccccagcgaggtggagcacatgttcagcccatcctgtgtctccctgctgcgctgcaccggctgctgcggcgatgagaatctgcactgtgtgccggtggagacggccaatgtcaccatgcagctcctaaagatccgttctggggaccggccctcctacgtggagctgacgttctctcagcacgttcgctgcgaatgccggcctctgcgggagaagatgaagccggaaaggtgcggcgatgctgttccccggaggtaacccaccccttggaggagagagaccccgcacccggctcgtgtatttattaccgtcacactcttcagtgactcctgctggtacctgccctctatttattagccaactgtttccctgctgaatgcctcgctcccttcaagacgaggggcagggaaggacaggaccctcaggaattcagtgccttcaacaacgtgagagaaagagagaagccagccacagacccctgggagcttccgctttgaaagaagcaagacacgtggcctcgtgaggggcaagctaggccccagaggccctggaggtctccaggggcctgcagaaggaaagaagggggccctgctacctgttcttgggcctcaggctctgcacagacaagcagcccttgctttcggagctcctgtccaaagtagggatgcggattctgctggggccgccacggcctggtggtgggaaggccggcagcgggcggaggggattcagccacttccccctcttcttctgaagatcagaacattcagctctggagaacagtggttgcctgggggcttttgccactccttgtcccccgtgatctcccctcacactttgccatttgcttgtactgggacattgttctttccggccgaggtgccaccaccctgcccccactaagagacacatacagagtgggccccgggctggagaaagagctgcctggatgagaaacagctcagccagtggggatgaggtcaccaggggaggagcctgtgcgtcccagctgaaggcagtggcaggggagcaggttccccaagggccctggcacccccacaagctgtccctgcagggccatctgactgccaagccagattctcttgaataaagtattctagtgtggaaacgc

SEQ ID NO: 85 - protein, 149 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERCGDAVPRR

SEQ ID NO: 86 - DNA, 2029 bp, Homo sapiens
  misc_feature 1..2029: note = VEGF-D
  source 1..2029: mol_type = genomic DNA
  CDS 411..1472
gttgggttccagctttctgtagctgtaagcattggtggccacaccacctccttacaaagcaactagaacctgcggcatacattggagagatttttttaattttctggacatgaagtaaatttagagtgctttctaatttcaggtagaagacatgtccaccttctgattatttttggagaacattttgatttttttcatctctctctccccacccctaagattgtgcaaaaaaagcgtaccttgcctaattgaaataatttcattggattttgatcagaactgattatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagccaaaaaaaaaaaaaaaaaa

SEQ ID NO: 87 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 88 - DNA, 1830 bp, ORF Virus
  misc_feature 1..1830: note = VEGF-E
  source 1..1830: mol_type = genomic DNA
  CDS 312..755
cggccacgcggccgcgaactgcgcgctcgcgcgcgtggcgaccgcgctgacgcgccgcgtgcccgcgagccggcacggcctcgcggagggcggcacgccgccgtggacgctgctgctggcggtggccgcggtggcggtgctcggcgtggtggcaatttcgctgctgcgccgcgcgctaagaatacggtttagatactcaaagtctatccagacacttagagtgtaactttgagtaaaaaatgtaaatactaacgccaaaatttcgatagttgttaagcaatatataacatttttaaaacgtcatcaccagcatgaagttaacagctacgttacaagttgttgttgcattgttaatatgtatgtataatttgccagaatgcgtgtctcagagtaatgattcacctccttcaaccaatgactggatgcgtacactagacaaaagtggttgtaaacctagagatactgttgtttatttgggagaagaatatccagaaagcactaacctacaatataatccccggtgcgtaactgttaaacgatgcagtggttgctgtaacggtgacggtcaaatatgtacagcggttgaaacaagaaatacaactgtaacagtttcagtaaccggcgtgtctagttcgtctggtactaatagtggtgtatctactaaccttcaaagaataagtgttacagaacacacaaagtgcgattgtattggtagaacaacgacaacacctacgaccactagggaacctagacgataactaataacaaaaaatgtttatttttgtaaatacttaattattacacactttacaataatctcaaaaataaattgcgtgcccggacggctgcagctggtgacgctgctgtgtcacacactgcgtattcgattcaagttcactaacgccactaaactagttgtgcgtgtccgagtgttaaccgtacgtcaaactaacatcttacctgtccgtgacaagaactaaaacttgaaccacatatttttaaagtatatttaacaaaatcactcacactcacacaatcataaacaccacaaccacaaccaaacacgcatgagaattaatattcttacttatccgtaacactctatgctgtacatcaacgcatcagagcagtctgagtctgactaatggcggcaaacgggaacgcaggcgcgacataatcactgagaatctccgcagcaaccgctcaaggacatctctagcgctaacggctgtttgtcattcccccgtgtgttcatctcacacgacattgtgaccgtcgcaaagcacacattcaaagtgccgcatgtggaagaattcaccgtcgagacacacaccataattaaacaagatcagtgcataagagagattagcattctacagcacaccacgtgcgaatacggacctcgtaattgtttagactagaacacctctggtctaaacaacatgtccgatcttagaacagagtttatgacgcatatgtaactgtgttctttatgtagaagttatcttttatgtcactcccttgtcttagatgagttatacatgacatgatgtatgtgtcgcccgcggcggcgcggggcgctcggcggcggggctgctgcgcgcggcgggcccgcggtggcggcggctggcgcggcgctgcggccgcgggcgcgcggcggggtagcggcccgcccgcccgggcgcccgccgcagcccttgccccggaccaggcgccacggagcaaagtgaaaaaggaccgcctagcagtcgagaccctcccgccgcagccgcgacaccccacacccgccttccacccgccagacgccaacaccacagccaacaagcatgc

SEQ ID NO: 89 - protein, 148 aa, ORF Virus
MKLTATLQVVVALLICMYNLPECVSQSNDSPPSTNDWMRTLDKSGCKPRDTVVYLGEEYPESTNLQYNPRCVTVKRCSGCCNGDGQICTAVETRNTTVTVSVTGVSSSSGTNSGVSTNLQRISVTEHTKCDCIGRTTTTPTTTREPRR

SEQ ID NO: 90 - DNA, 815 bp, Homo sapiens
  misc_feature 1..815: note = 232 amino acid isoform of VEGF-A
  source 1..815: mol_type = genomic DNA
  CDS 69..767
gaattcgaattccagtgtgctggcggccgcgcgcgagccgcgccggccccggtcgggcctccgaaaccatgaactttctgctgtcttgggtgcattggagcctcgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggctggaggaaggagcctccctcagggtttcgggaaccagatcc

SEQ ID NO: 91 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 92 - DNA, 399 bp, ORF virus
  misc_feature 1..399: note = D1701 VEGF
  source 1..399: mol_type = genomic DNA
  CDS 1..399
atgaagtttctcgtcggcatactggtagctgtgtgcttgcaccagtatctgctgaacgcggacagcacgaaaacatggtccgaagtgtttgaaaacagcgggtgcaagccaaggccgatggtctttcgagtacacgacgagcacccggagctaacttctcagcggttcaacccgccgtgtgtcacgttgatgcgatgcggcgggtgctgcaacgacgagagcttagaatgcgtccccacggaagaggcaaacgtaacgatgcaactcatgggagcgtcggtctccggtggtaacgggatgcaacatctgagcttcgtagagcataagaaatgcgattgtaaaccaccactcacgaccacgccaccgacgaccacaaggccgcccagaagacgccgctag

SEQ ID NO: 93 - protein, 132 aa, ORF virus
MKFLVGILVAVCLHQYLLNADSTKTWSEVFENSGCKPRPMVFRVHDEHPELTSQRFNPPCVTLMRCGGCCNDESLECVPTEEANVTMQLMGASVSGGNGMQHLSFVEHKKCDCKPPLTTTPPTTTRPPRRRR

SEQ ID NO: 94 - DNA, 570 bp, Homo sapiens
  misc_feature 1..570: note = VEGF-B Isoform 1
  source 1..570: mol_type = genomic DNA
accatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtga

SEQ ID NO: 95 - protein, 188 aa, Homo sapiens
  REGION 1..188: note = MISC_FEATURE - VEGF-B Isoform 1
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDSPRPLCPRCTQHHQRPDPRTCRCRCRRRSFLRCQGRGLELNPDTCRCRKLRR

SEQ ID NO: 96 - DNA, 624 bp, Homo sapiens
  misc_feature 1..624: note = VEGF-B Isoform 2
  source 1..624: mol_type = genomic DNA
atgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgccgccgctgccgacgccgcagcttcctccgttgccaagggcggggcttag

SEQ ID NO: 97 - protein, 207 aa, Homo sapiens
  REGION 1..207: note = MISC_FEATURE - VEGF-B Isoform 2
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 98 - DNA, 2305 bp, Homo sapiens
  misc_feature 1..2305: note = PDGF-A
  source 1..2305: mol_type = genomic DNA
  CDS 404..991: note = PDGF-A
ttcttggggctgatgtccgcaaatatgcagaattaccggccgggtcgctcctgaagccagcgcggggagcgagcgcggcggcggccagcaccgggaacgcaccgaggaagaagcccagcccccgccctccgccccttccgtccccaccccctacccggcggcccaggaggctccccggctgcggcgcgcactccctgtttctcctcctcctggctggcgctgcctgcctctccgcactcactgctcgccgggcgccgtccgccagctccgtgctccccgcgccaccctcctccgggccgcgctccctaagggatggtactgaatttcgccgccacaggagaccggctggagcgcccgccccgcgcctcgcctctcctccgagcagccagcgcctcgggacgcgatgaggaccttggcttgcctgctgctcctcggctgcggatacctcgcccatgttctggccgaggaagccgagatcccccgcgaggtgatcgagaggctggcccgcagtcagatccacagcatccgggacctccagcgactcctggagatagactccgtagggagtgaggattctttggacaccagcctgagagctcacggggtccacgccactaagcatgtgcccgagaagcggcccctgcccattcggaggaagagaagcatcgaggaagctgtccccgctgtctgcaagaccaggacggtcatttacgagattcctcggagtcaggtcgaccccacgtccgccaacttcctgatctggcccccgtgcgtggaggtgaaacgctgcaccggctgctgcaacacgagcagtgtcaagtgccagccctcccgcgtccaccaccgcagcgtcaaggtggccaaggtggaatacgtcaggaagaagccaaaattaaaagaagtccaggtgaggttagaggagcatttggagtgcgcctgcgcgaccacaagcctgaatccggattatcgggaagaggacacggatgtgaggtgaggatgagccgcagccctttcctgggacatggatgtacatggcgtgttacattcctgaacctactatgtacggtgctttattgccagtgtgcggtctttgttctcctccgtgaaaaactgtgtccgagaacactcgggagaacaaagagacagtgcacatttgtttaatgtgacatcaaagcaagtattgtagcactcggtgaagcagtaagaagcttccttgtcaaaaagagagagagagagagagagagagaaaacaaaaccacaaatgacaaaaacaaaacggactcacaaaaatatctaaactcgatgagatggagggtcgccccgtgggatggaagtgcagaggtctcagcagactggatttctgtccgggtggtcacaggtgcttttttgccgaggatgcagagcctgctttgggaacgactccagaggggtgctggtgggctctgcagggcccgcaggaagcaggaatgtcttggaaaccgccacgcgaactttagaaaccacacctcctcgctgtagtatttaagcccatacagaaaccttcctgagagccttaagtggtttttttttttgtttttgttttgttttttttttttttgttttttttttttttttttttttttacaccataaagtgattattaagcttccttttactctttggctagctttttttttttttttttttttttttttttttaattatctcttggatgacatttacaccgataacacacaggctgctgtaactgtcaggacagtgcgacggtatttttcctagcaagatgcaaactaatgagatgtattaaaataaacatggtatacctacctatgcatcatttcctaaatgtttctggctttgtgtttctcccttaccctgctttatttgttaatttaagccattttgaaagaactatgcgtcaaccaatcgtacgccgtccctgcggcacctgccccagagcccgtttgtggctgagtgacaacttgttccccgcagtgcacacctagaatgctgtgttcccacgcggcacgtgagatgcattgccgcttctgtctgtgttgttggtgtgccctggtgccgtggtggcggtcactccctctgctgccagtgtttggacagaacccaaattctttatttttggtaagatattgtgctttacctgtattaacagaaatgtgtgtgtgtggtttgtttttttgtaaaggtgaagtttgtatgtttacctaatattacctgttttgtatacctgagagcctgctatgttcttcttttgttgatccaaaattaaaaaaaaaataccaccaac

SEQ ID NO: 99 - protein, 196 aa, Homo sapiens
MRTLACLLLLGCGYLAHVLAEEAEIPREVIERLARSQIHSIRDLQRLLEIDSVGSEDSLDTSLRAHGVHATKHVPEKRPLPIRRKRSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTDVR

SEQ ID NO: 100 - DNA, 2137 bp, Homo sapiens
  misc_feature 1..2137: note = PDGF-B
  source 1..2137: mol_type = genomic DNA
  CDS 983..1705
ccctgcctgcctccctgcgcacccgcagcctcccccgctgcctccctagggctcccctccggccgccagcgcccatttttcattccctagatagagatactttgcgcgcacacacatacatacgcgcgcaaaaaggaaaaaaaaaaaaaaaagcccaccctccagcctcgctgcaaagagaaaaccggagcagccgcagctcgcagctcgcagcccgcagcccgcagaggacgcccagagcggcgagcgggcgggcagacggaccgacggactcgcgccgcgtccacctgtcggccgggcccagccgagcgcgcagcgggcacgccgcgcgcgcggagcagccgtgcccgccgcccgggcccgccgccagggcgcacacgctcccgcccccctacccggcccgggcgggagtttgcacctctccctgcccgggtgctcgagctgccgttgcaaagccaactttggaaaaagttttttgggggagacttgggccttgaggtgcccagctccgcgctttccgattttgggggcctttccagaaaatgttgcaaaaaagctaagccggcgggcagaggaaaacgcctgtagccggcgagtgaagacgaaccatcgactgccgtgttccttttcctcttggaggttggagtcccctgggcgcccccacacggctagacgcctcggctggttcgcgacgcagccccccggccgtggatgctgcactcgggctcgggatccgcccaggtagcggcctcggacccaggtcctgcgcccaggtcctcccctgccccccagcgacggagccggggccgggggcggcggcgccgggggcatgcgggtgagccgcggctgcagaggcctgagcgcctgatcgccgcggacccgagccgagcccacccccctccccagccccccaccctggccgcgggggcggcgcgctcgatctacgcgttcggggccccgcggggccgggcccggagtcggcatgaatcgctgctgggcgctcttcctgtctctctgctgctacctgcgtctggtcagcgccgagggggaccccattcccgaggagctttatgagatgctgagtgaccactcgatccgctcctttgatgatctccaacgcctgctgcacggagaccccggagaggaagatggggccgagttggacctgaacatgacccgctcccactctggaggcgagctggagagcttggctcgtggaagaaggagcctgggttccctgaccattgctgagccggccatgatcgccgagtgcaagacgcgcaccgaggtgttcgagatctcccggcgcctcatagaccgcaccaacgccaacttcctggtgtggccgccctgtgtggaggtgcagcgctgctccggctgctgcaacaaccgcaacgtgcagtgccgccccacccaggtgcagctgcgacctgtccaggtgagaaagatcgagattgtgcggaagaagccaatctttaagaaggccacggtgacgctggaagaccacctggcatgcaagtgtgagacagtggcagctgcacggcctgtgacccgaagcccggggggttcccaggagcagcgagccaaaacgccccaaactcgggtgaccattcggacggtgcgagtccgccggccccccaagggcaagcaccggaaattcaagcacacgcatgacaagacggcactgaaggagacccttggagcctaggggcatcggcaggagagtgtgtgggcagggttatttaatatggtatttgctgtattgcccccatggggccttggagtagataatattgtttccctcgtccgtctgtctcgatgcctgattcggacggccaatggtgcctcccccacccctccacgtgtccgtccacccttccatcagcgggtctcctcccagcggcctccggctcttgcccagcagctcaagaagaaaaagaaggactgaactccatcgccatcttcttcccttaactccaagaacttgggataagagtgtgagagagactgatggggtcgctctttgggggaaacgggttccttcccctgcacctggcctgggccacacctgagcgctgtggactgtcctgaggagccctgaggacctctcagcatagcctgcctgatccctgaaccc

SEQ ID NO: 101 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 102 - DNA, 2108 bp, Homo sapiens
  misc_feature 1..2108: note = PDGF-C
  misc_feature 1..2108: note = n = a, c, g, or t
  misc_feature 1..2108: note = n = a, c, g, or t
  misc_feature 1..2108: note = n = a, c, g, or t
  misc_feature 1..2108: note = n = a, c, g, or t
  misc_feature 2002: note = n is a, c, g, or t
  misc_feature 2065: note = n is a, c, g, or t
  misc_feature 2070: note = n is a, c, g, or t
  misc_feature 2089: note = n is a, c, g, or t
  source 1..2108: mol_type = genomic DNA
ccccgccgtgagtgagctctcaccccagtcagccaaatgagcctcttcgggcttctcctggtgacatctgccctggccggccagagacgagggactcaggcggaatccaacctgagtagtaaattccagttttccagcaacaaggaacagaacggagtacaagatcctcagcatgagagaattattactgtgtctactaatggaagtattcacagcccaaggtttcctcatacttatccaagaaatacggtcttggtatggagattagtagcagtagaggaaaatgtatggatacaacttacgtttgatgaaagatttgggcttgaagacccagaagatgacatatgcaagtatgattttgtagaagttgaggaacccagtgatggaactatattagggcgctggtgtggttctggtactgtaccaggaaaacagatttctaaaggaaatcaaattaggataagatttgtatctgatgaatattttccttctgaaccagggttctgcatccactacaacattgtcatgccacaattcacagaagctgtgagtccttcagtgctacccccttcagctttgccactggacctgcttaataatgctataactgcctttagtaccttggaagaccttattcgatatcttgaaccagagagatggcagttggacttagaagatctatataggccaacttggcaacttcttggcaaggcttttgtttttggaagaaaatccagagtggtggatctgaaccttctaacagaggaggtaagattatacagctgcacacctcgtaacttctcagtgtccataagggaagaactaaagagaaccgataccattttctggccaggttgtctcctggttaaacgctgtggtgggaactgtgcctgttgtctccacaattgcaatgaatgtcaatgtgtcccaagcaaagttactaaaaaataccacgaggtccttcagttgagaccaaagaccggtgtcaggggattgcacaaatcactcaccgacgtggccctggagcaccatgaggagtgtgactgtgtgtgcagagggagcacaggaggatagccgcatcaccaccagcagctcttgcccagagctgtgcagtgcagtggctgattctattagagaacgtatgcgttatctccatccttaatctcagttgtttgcttcaaggacctttcatcttcaggatttacagtgcattctgaaagaggagacatcaaacagaattaggagttgtgcaacagctcttttgagaggaggcctaaaggacaggagaaaaggtcttcaatcgtggaaagaaaattaaatgttgtattaaatagatcaccagctagtttcagagttaccatgtacgtattccactagctgggttctgtatttcagttctttcgatacggcttagggtaatgtcagtacaggaaaaaaactgtgcaagtgagcacctgattccgttgccttgcttaactctaaagctccatgtcctgggcctaaaatcgtataaaatctggatttttttttttttttttgctcatattcacatatgtaaaccagaacattctatgtactacaaacctggtttttaaaaaggaactatgttgctatgaattaaacttgtgtcrtgctgataggacagactggatttttcatatttcttattaaaatttctgccatttagaagaagagaactacattcatggtttggaagagataaacctgaaaagaagagtggccttatcttcactttatcgataagtcagtttatttgtttcattgtgtacatttttatattctccttttgacattataactgttggcttttctaatcttgttaaatatatctatttttaccaaaggtatttaatattcttttttatgacaacttagatcaactatttttagcttggtaaatttttctaaacacaattgttatagccagaggaacaaagatggatataaaaatattgttgccctggacaaaaatacatgtatntccatcccggaatggtgctagagttggattaaacctgcattttaaaaaacctgaattgggaanggaanttggtaaggttggccaaancttttttgaaaataattaa

SEQ ID NO: 103 - protein, 345 aa, Homo sapiens [sequence omitted]

SEQ ID NO: 104 - DNA, 2253 bp, Homo sapiens
  misc_feature 1..2253: note = PDGF-D
  source 1..2253: mol_type = genomic DNA
cgctcggaaagttcagcatgcaggaagtttggggagagctcggcgattagcacagcgacccgggccagcgcagggcgagcgcaggcggcgagagcgcagggcggcgcggcgtcggtcccgggagcagaacccggctttttcttggagcgacgctgtctctagtcgctgatcccaaatgcaccggctcatctttgtctacactctaatctgcgcaaacttttgcagctgtcgggacacttctgcaaccccgcagagcgcatccatcaaagctttgcgcaacgccaacctcaggcgagatgagagcaatcacctcacagacttgtaccgaagagatgagaccatccaggtgaaaggaaacggctacgtgcagagtcctagattcccgaacagctaccccaggaacctgctcctgacatggcggcttcactctcaggagaatacacggatacagctagtgtttgacaatcagtttggattagaggaagcagaaaatgatatctgtaggtatgattttgtggaagttgaagatatatccgaaaccagtaccattattagaggacgatggtgtggacacaaggaagttcctccaaggataaaatcaagaacgaaccaaattaaaatcacattcaagtccgatgactactttgtggctaaacctggattcaagatttattattctttgctggaagatttccaacccgcagcagcttcagagaccaactgggaatctgtcacaagctctatttcaggggtatcctataactctccatcagtaacggatcccactctgattgcggatgctctggacaaaaaaattgcagaatttgatacagtggaagatctgctcaagtacttcaatccagagtcatggcaagaagatcttgagaatatgtatctggacacccctcggtatcgaggcaggtcataccatgaccggaagtcaaaagttgacctggataggctcaatgatgatgccaagcgttacagttgcactcccaggaattactcggtcaatataagagaagagctgaagttggccaatgtggtcttctttccacgttgcctcctcgtgcagcgctgtggaggaaattgtggctgtggaactgtcaactggaggtcctgcacatgcaattcagggaaaaccgtgaaaaagtatcatgaggtattacagtttgagcctggccacatcaagaggaggggtagagctaagaccatggctctagttgacatccagttggatcaccatgaacgatgcgattgtatctgcagctcaagaccacctcgataagagaatgtgcacatccttacattaagcctgaaagaacctttagtttaaggagggtgagataagagacccttttcctaccagcaaccaaacttactactagcctgcaatgcaatgaacacaagtggttgctgagtctcagccttgctttgttaatgccatggcaagtagaaaggtatatcatcaacttctatacctaagaatataggattgcatttaataatagtgtttgaggttatatatgcacaaacacacacagaaatatattcatgtctatgtgtatatagatcaaatgttttttttggtatatataaccaggtacaccagagcttacatatgtttgagttagactcttaaaatcctttgccaaaataagggatggtcaaatatatgaaacatgtctttagaaaatttaggagataaatttatttttaaattttgaaacacaaaacaattttgaatcttgctctcttaaagaaagcatcttgtatattaaaaatcaaaagatgaggctttcttacatatacatcttagttgattattaaaaaaggaaaaaggtttccagagaaaaggccaatacctaagcattttttccatgagaagcactgcatacttacctatgtggactgtaataacctgtctccaaaaccatgccataataatataagtgctttagaaattaaatcattgtgttttttatgcattttgctgaggcatccttattcatttaacacctatctcaaaaacttacttagaaggttttttattatagtcctacaaaagacaatgtataagctgtaacagaattttgaattgtttttctttgcaaaacccctccacaaaagcaaatcctttcaagaatggcatgggcattctgtatgaacctttccagatggtgttcagtgaaagatgtgggtagttgagaacttaaaaagtgaacattgaaacatcgacgtaactggaaaccg

SEQ ID NO: 105 - protein, 116 aa, Homo sapiens
  REGION 1..116: note = MISC_FEATURE - PDGF-D
GRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

SEQ ID NO: 106 - DNA, 456 bp, synthetic construct
  misc_feature 1..456: note = Synthetic construct
  misc_feature 1..456: note = hVEGFA109
  source 1..456: mol_type = other DNA
atggagacagacacactcctgctatgggtactgctgctctgggttccaggttccactggtgacgcggcccaggatcctgggcagaatcatcacgaagtggtgaaattcatggatgtctatcagcgcagctactgccatccgatcgagacactggtggacatcttccaggaataccctgatgagatcgagtacatcttcaagccatcctgcgtgcccctgatgagatgtgggggttgctgcaatgacgaagggctggagtgcgttcccaccgaggagtccaacatcaccatgcagattatgagaattaaacctcaccaagggcagcacatcggagagatgagctttctccagcataacaaatgtgaatgtagaccaaagaaagatttggtcttcgaacaaaaactcatctcagaagaggatctgaatagcgccgtcgaccatcatcatcatcatcat

SEQ ID NO: 107 - protein, 152 aa, synthetic construct
  REGION 1..152: note = Synthetic construct
  REGION 1..152: note = MISC_FEATURE - hVEGFA109
METDTLLLWVLLLWVPGSTGDAAQDPGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDLVFEQKLISEEDLNSAVDHHHHHH

SEQ ID NO: 108 - DNA, 504 bp, synthetic construct
  misc_feature 1..504: note = Synthetic construct
  misc_feature 1..504: note = hVEGFC109
  source 1..504: mol_type = other DNA
atggagacagacacactcctgctatgggtactgctgctctgggttccaggttccactggtgacgcggcccagccggccaggcgcgccgtacgaagcttggtaccgagctcggatccagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaagctggatttggtcttcgaacaaaaactcatctcagaagaggatctgaatagcgccgtcgaccatcatcatcatcatcat

SEQ ID NO: 109 - protein, 168 aa, synthetic construct
  REGION 1..168: note = Synthetic construct
  REGION 1..168: note = MISC_FEATURE - hVEGFC109
METDTLLLWVLLLWVPGSTGDAAQPARRAVRSLVPSSDPAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDLVFEQKLISEEDLNSAVDHHHHHH

SEQ ID NO: 110 - protein, 87 aa, synthetic construct
  REGION 1..87: note = Synthetic construct
  REGION 1..87: note = MISC_FEATURE - VHD motif
  REGION 1..87: note = misc_feature - Xaa = any or unknown amino acid
  REGION 1..87: note = misc_feature - Xaa = any amino acid or unknown amino acid or nothing
  REGION 1..87: note = misc_feature - Xaa = Proline, Serine or Arginine
  REGION 1..87: note = misc_feature - Xaa = any or unknown amino acid
  REGION 1..87: note = misc_feature - Xaa = Guanine, Serine, Threonine or Alanine
  REGION 1..87: note = misc_feature - Xaa = any or unknown amino acid
  REGION 1..87: note = misc_feature - Xaa = any or unknown amino acid
  REGION 1..87: note = misc_feature - Xaa = any amino acid or unknown amino acid or nothing
  REGION 2..25: note = misc_feature - Xaa can be any naturally occurring amino acid
  SITE 27: note = misc_feature - Xaa can be any naturally occurring amino acid
  REGION 30..32: note = misc_feature - Xaa can be any naturally occurring amino acid
  SITE 35: note = misc_feature - Xaa can be any naturally occurring amino acid
  REGION 39..44: note = misc_feature - Xaa can be any naturally occurring amino acid
  REGION 46..86: note = misc_feature - Xaa can be any naturally occurring amino acid
CXXXXXXXXXXXXXXXXXXXXXXXXPXCVXXXRCXGCCXXXXXXCXXXXXXXXXXXXXXXXXXXXXXXXXXXXXXXXXXXXXXXXXC

SEQ ID NO: 111 - protein, 13 aa, synthetic construct
  REGION 1..13: note = Synthetic construct
  REGION 1..13: note = MISC_FEATURE - PDGF motif
  REGION 1..13: note = misc_feature - Xaa = Proline or Serine
  REGION 1..13: note = misc_feature - Xaa = any or unknown amino acid
  REGION 1..13: note = misc_feature - Xaa = Arginine, Serine, Threonine or Alanine
  SITE 2: note = misc_feature - Xaa can be any naturally occurring amino acid
  REGION 5..7: note = misc_feature - Xaa can be any naturally occurring amino acid
  SITE 10: note = misc_feature - Xaa can be any naturally occurring amino acid
PXCVXXXRCXGCC

SEQ ID NO: 112 - DNA, 2772 bp, Homo sapiens
  source 1..2772: mol_type = genomic DNA
  CDS 1..2772
atggagagggggctgccgctcctctgcgccgtgctcgccctcgtcctcgccccggccggcgcttttcgcaacgatgaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaaattttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccccacagatgttgtggttgcagtattccccaaaccactgataactcgatttgtccgaatcaagcctgcaacttgggaaactggcatatctatgagatttgaagtatacggttgcaagataacagattatccttgctctggaatgttgggtatggtgtctggacttatttctgactcccagatcacatcatccaaccaaggagacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaacccatcctcatcaccatcatagccatgagcgccctgggggtcctcctgggggctgtctgtggggtcgtgctgtactgtgcctgttggcataatgggatgtcagaaagaaacttgtctgccctggagaactataactttgaacttgtggatggtgtgaagttgaaaaaagacaaactgaatacacagagtacttattcggaggcatga

SEQ ID NO: 113 - protein, 923 aa, Homo sapiens
MERGLPLLCAVLALVLAPAGAFRNDECGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLEPILITIIAMSALGVLLGAVCGVVLYCACWHNGMSERNLSALENYNFELVDGVKLKKDKLNTQSTYSEA

SEQ ID NO: 114 - DNA, 2781 bp, Homo sapiens
  source 1..2781: mol_type = genomic DNA
  CDS 1..2781
atggatatgtttcctctcacctgggttttcttagccctctacttttcaagacaccaagtgagaggccaaccagacccaccgtgcggaggtcgtttgaattccaaagatgctggctatatcacctctcccggttacccccaggactacccctcccaccagaactgcgagtggattgtttacgcccccgaacccaaccagaagattgtcctcaacttcaaccctcactttgaaatcgagaagcacgactgcaagtatgactttatcgagattcgggatggggacagtgaatccgcagacctcctgggcaaacactgtgggaacatcgccccgcccaccatcatctcctcgggctccatgctctacatcaagttcacctccgactacgcccggcagggggcaggcttctctctgcgctacgagatcttcaagacaggctctgaagattgctcaaaaaacttcacaagccccaacgggaccatcgaatctcctgggtttcctgagaagtatccacacaacttggactgcacctttaccatcctggccaaacccaagatggagatcatcctgcagttcctgatctttgacctggagcatgaccctttgcaggtgggagagggggactgcaagtacgattggctggacatctgggatggcattccacatgttggccccctgattggcaagtactgtgggaccaaaacaccctctgaacttcgttcatcgacggggatcctctccctgacctttcacacggacatggcggtggccaaggatggcttctctgcgcgttactacctggtccaccaagagccactagagaactttcagtgcaatgttcctctgggcatggagtctggccggattgctaatgaacagatcagtgcctcatctacctactctgatgggaggtggacccctcaacaaagccggctccatggtgatgacaatggctggacccccaacttggattccaacaaggagtatctccaggtggacctgcgctttttaaccatgctcacggccatcgcaacacagggagcgatttccagggaaacacagaatggctactacgtcaaatcctacaagctggaagtcagcactaatggagaggactggatggtgtaccggcatggcaaaaaccacaaggtatttcaagccaacaacgatgcaactgaggtggttctgaacaagctccacgctccactgctgacaaggtttgttagaatccgccctcagacctggcactcaggtatcgccctccggctggagctcttcggctgccgggtcacagatgctccctgctccaacatgctggggatgctctcaggcctcattgcagactcccagatctccgcctcttccacccaggaatacctctggagccccagtgcagcccgcctggtcagcagccgctcgggctggttccctcgaatccctcaggcccagcccggtgaggagtggcttcaggtagatctgggaacacccaagacagtgaaaggtgtcatcatccagggagcccgcggaggagacagtatcactgctgtggaagccagagcatttgtgcgcaagttcaaagtctcctacagcctaaacggcaaggactgggaatacattcaggaccccaggacccagcagccaaagctgttcgaagggaacatgcactatgacacccctgacatccgaaggtttgaccccattccggcacagtatgtgcgggtatacccggagaggtggtcgccggcggggattgggatgcggctggaggtgctgggctgtgactggacagactccaagcccacggtaaaaacgctgggacccactgtgaagagcgaagagacaaccaccccctaccccaccgaagaggaggccacagagtgtggggagaactgcagctttgaggatgacaaagatttgcagctcccttcgggattcaattgcaacttcgatttcctcgaggagccctgtggttggatgtatgaccatgccaagtggctccggaccacctgggccagcagctccagcccaaacgaccggacgtttccagatgacaggaatttcttgcggctgcagagtgacagccagagagagggccagtatgcccggctcatcagcccccctgtccacctgccccgaagcccggtgtgcatggagttccagtaccaggccacgggcggccgcggggtggcgctgcaggtggtgcgggaagccagccaggagagcaagttgctgtgggtcatccgtgaggaccagggcggcgagtggaagcacgggcggatcatcctgcccagctacgacatggagtaccagattgtgttcgagggagtgatagggaaaggacgttccggagagattgccattgatgacattcggataagcactgatgtcccactggagaactgcatggaacccatctcggcttttgcagtggacatcccagaaatacatgagagagaaggatatgaagatgaaattgatgatgaatacgaggtggactggagcaattcttcttctgcaacctcagggtctggcgccccctcgaccgacaaagaaaagagctggctgtacaccctggatcccatcctcatcaccatcatcgccatgagctcactgggcgtcctcctgggggccacctgtgcaggcctcctgctctactgcacctgttcctactcgggcctgagctcccgaagctgcaccacactggagaactacaacttcgagctctacgatggccttaagcacaaggtcaagatgaaccaccaaaagtgctgctccgaggcatga

SEQ ID NO: 115 - protein, 926 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVKTLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 116 - DNA, 6375 bp, Homo sapiens
  misc_feature 1..6375: note = n is a, c, g, or t
  misc_feature 1..6375: note = n is a, c, g, or t
  misc_feature 4476: note = n is a, c, g, or t
  misc_feature 4499: note = n is a, c, g, or t
  source 1..6375: mol_type = genomic DNA
  CDS 129..3398
ttggagctacagggagagaaacagaggaggagactgcaagagatcattggaggccgtgggcacgctctttactccatgtgtgggacattcattgcggaataacatcggaggagaagtttcccagagctatggggacttcccatccggcgttcctggtcttaggctgtcttctcacagggctgagcctaatcctctgccagctttcattaccctctatccttccaaatgaaaatgaaaaggttgtgcagctgaattcatccttttctctgagatgctttggggagagtgaagtgagctggcagtaccccatgtctgaagaagagagctccgatgtggaaatcagaaatgaagaaaacaacagcggcctttttgtgacggtcttggaagtgagcagtgcctcggcggcccacacagggttgtacacttgctattacaaccacactcagacagaagagaatgagcttgaaggcaggcacatttacatctatgtgccagacccagatgtagcctttgtacctctaggaatgacggattatttagtcatcgtggaggatgatgattctgccattataccttgtcgcacaactgatcccgagactcctgtaaccttacacaacagtgagggggtggtacctgcctcctacgacagcagacagggctttaatgggaccttcactgtagggccctatatctgtgaggccaccgtcaaaggaaagaagttccagaccatcccatttaatgtttatgctttaaaagcaacatcagagctggatctagaaatggaagctcttaaaaccgtgtataagtcaggggaaacgattgtggtcacctgtgctgtttttaacaatgaggtggttgaccttcaatggacttaccctggagaagtgaaaggcaaaggcatcacaatgctggaagaaatcaaagtcccatccatcaaattggtgtacactttgacggtccccgaggccacggtgaaagacagtggagattacgaatgtgctgcccgccaggctaccagggaggtcaaagaaatgaagaaagtcactatttctgtccatgagaaaggtttcattgaaatcaaacccaccttcagccagttggaagctgtcaacctgcatgaagtcaaacattttgttgtagaggtgcgggcctacccacctcccaggatatcctggctgaaaaacaatctgactctgattgaaaatctcactgagatcaccactgatgtggaaaagattcaggaaataaggtatcgaagcaaattaaagctgatccgtgctaaggaagaagacagtggccattatactattgtagctcaaaatgaagatgctgtgaagagctatacttttgaactgttaactcaagttccttcatccattctggacttggtcgatgatcaccatggctcaactgggggacagacggtgaggtgcacagctgaaggcacgccgcttcctgatattgagtggatgatatgcaaagatattaagaaatgtaataatgaaacttcctggactattttggccaacaatgtctcaaacatcatcacggagatccactcccgagacaggagtaccgtggagggccgtgtgactttcgccaaagtggaggagaccatcgccgtgcgatgcctggctaagaatctccttggagctgagaaccgagagctgaagctggtggctcccaccctgcgttctgaactcacggtggctgctgcagtcctggtgctgttggtgattgtgatcatctcacttattgtcctggttgtcatttggaaacagaaaccgaggtatgaaattcgctggagggtcattgaatcaatcagcccagatggacatgaatatatttatgtggacccgatgcagctgccttatgactcaagatgggagtttccaagagatggactagtgcttggtcgggtcttggggtctggagcgtttgggaaggtggttgaaggaacagcctatggattaagccggtcccaacctgtcatgaaagttgcagtgaagatgctaaaacccacggccagatccagtgaaaaacaagctctcatgtctgaactgaagataatgactcacctggggccacatttgaacattgtaaacttgctgggagcctgcaccaagtcaggccccatttacatcatcacagagtattgcttctatggagatttggtcaactatttgcataagaatagggatagcttcctgagccaccacccagagaagccaaagaaagagctggatatctttggattgaaccctgctgatgaaagcacacggagctatgttattttatcttttgaaaacaatggtgactacatggacatgaagcaggctgatactacacagtatgtccccatgctagaaaggaaagaggtttctaaatattccgacatccagagatcactctatgatcgtccagcctcatataagaagaaatctatgttagactcagaagtcaaaaacctcctttcagatgataactcagaaggccttactttattggatttgttgagcttcacctatcaagttgcccgaggaatggagtttttggcttcaaaaaattgtgtccaccgtgatctggctgctcgcaacgttctcctggcacaaggaaaaattgtgaagatctgtgactttggcctggccagagacatcatgcatgattcgaactatgtgtcgaaaggcagtacctttctgcccgtgaagtggatggctcctgagagcatctttgacaacctctacaccacactgagtgatgtctggtcttatggcattctgctctgggagatcttttcccttggtggcaccccttaccccggcatgatggtggattctactttctacaataagatcaagagtgggtaccggatggccaagcctgaccacgctaccagtgaagtctacgagatcatggtgaaatgctggaacagtgagccggagaagagaccctccttttaccacctgagtgagattgtggagaatctgctgcctggacaatataaaaagagttatgaaaaaattcacctggacttcctgaagagtgaccatcctgctgtggcacgcatgcgtgtggactcagacaatgcatacattggtgtcacctacaaaaacgaggaagacaagctgaaggactgggagggtggtctggatgagcagagactgagcgctgacagtggctacatcattcctctgcctgacattgaccctgtccctgaggaggaggacctgggcaagaggaacagacacagctcgcagacctctgaagagagtgccattgagacgggttccagcagttccaccttcatcaagagagaggacgagaccattgaagacatcgacatgatggacgacatcggcatagactcttcagacctggtggaagacagcttcctgtaactggcggattcgaggggttccttccacttctggggccacctctggatcccgttcagaaaaccactttattgcaatgcggaggttgagaggaggacttggttgatgtttaaagagaagttcccagccaagggcctcggggagcctttctaaatatgaatgaatgggatattttgaaatgaactttgtcagtgttgcctcttgcaatgcctcagtagcatctcagtggtgtgtgaagtttggagatagatggataagggaataataggccacagaaggtgaactttctgcttcaaggacattggtgagagtccaacagacacaatttatactgcgacagaacttcagcattgtaattatgtaaataactctaaccacggctgtgtttagattgtattaactatcttctttggacttctgaagagaccactcaatccatccatgtacttccctcttgaaacctgatgtcagctgctgttgaactttttaaagaagtgcatgaaaaaccatttttgaccttaaaaggtactggtactatagcattttgctatcttttttagtgttaaagagataaagaataataattaaccaaccttgtttaatagatttgggtcatttagaagcctgacaactcattttcatattgtaatctatgtttataatactactactgttatcagtaatgctaaatgtgtaataatgtaacatgatttccctccacacaaagcacaatttaaaaacaatccttactaagtaggtgatgagtttgacagtttttgacatttatattaaataacatgtttctctataaagtatggtaatagctttagtgaattaaatttagttgagcatagagaacaaagtaaaagtagtgttgtccaggaagtcagaatttttaactgtactgaataggttccccaatccatcgtattaaaaaacaattaactgccctctgaaataatgggattagaaacaaacaaaactcttaagtcctaaaagttctcaatgtagaggcataaacctgtgctgaacataacttctcatgtatattacccaatggaaaatataatgatcagcgcanaaagactggatttgcagaagttnttttttttttttcttcttgcctgatgaaagctttggcgaccccaatatatgtattttttgaatctatgaacctgaaaagggtcacaaaggatgcccagacatcagcctccttctttcaccccttaccccaaagagaaagagtttgaaactcgagaccataaagatattctttagtggaggctggaagtgcattagcctgatcctcagttctcaaatgtgtgtggcagccaggtagactagtacctgggtttccatccttgagattctgaagtatgaagtctgagggaaaccagagtctgtatttttctaaactccctggctgttctgatcggccaggtttcggaaacactgacttaggtttcaggaagttgccatgggaaacaaataatttgaactttggaacagggttcttaagttggtgcgtccttcggatgataaatttaggaaccgaagtccaatcactgtaaattacggtagatcgatcgttaacgctggaattaaattgaaaggtcagaatcgactccgactctttcgatttcaaaccaaaactgtccaaaaggttttcatttctacgatgaagggtgacataccccctctaacttgaaaggggcagagggcagaagagcggagggtgaggtatggggcggttcctttccgtacatgtttttaatacgttaagtcacaaggttcagagacacattggtcgagtcacaaaaccaccttttttgtaaaattcaaaatgactattaaactccaatctaccctcctacttaacagtgtagataggtgtgacagtttgtccaaccacacccaagtaaccgtaagaaacgttatgacgaattaacgactatggtatacttactttgtacccgacactaatgacgttagtgacacgatagccgtctactacgaaaccttctacgtcttcgttattatttcatgaactgatggatgaccacattagagttacgttcggggttgaaagaataggttgaaaaagtatcattcacgcttctgactcggtctaaccggttaatttttcttttggactgatccaagacatctcggttaatctgaactttatgcaaacacaaagatcttagtgtcgagttcgtaagacaaatagcgagtgagagggaacatgtcggaataaaacaaccacgaaacgtaaaactataacgacactcggaacgtactgtagtactccggcctactttgaagagtcaggtcgtcaaaggtcaggattgtttacgagggtggacttaaacatatactgacgtaaacacccacacacacacaaaagtcgtttaaggtctaaacaaaggaaaaccggaggacgtttcagaggtcttcttttaaacggttagaaaggatgaaagataaaaatactactgttagtttcggccggactctttgtgataaacactgaaaaatttgctaatcactacaggaattttacaccagacggttagacatgttttaccaggataaaaacacttctccctgtattctattttactacaatatgtagttatacatatatacataaagatatatctgaacctcttatgacggttttgtaaatactgttcgacatagtgacggaagcaaatataaaaaaattgacactattaggggtgtccgtgtaattgacaacgtgaaaacttacaggttttaaatataaaatctttattatttttctttctatgaatgtacaagggttttgttaccacaccacttacacactctttttgattgaactatcccagatggttatgttttacataatgcttacggggacaagtacaaaaacaaaattttgcacatttacttctagaaatataaagttatttactatatattaaatttccttaag

SEQ ID NO: 117 - protein, 1089 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 118 - DNA, 5427 bp, Homo sapiens
  source 1..5427: mol_type = genomic DNA
  CDS 187..3507
tgttctcctgagccttcaggagcctgcaccagtcctgcctgtccttctactcagctgttacccactctgggaccagcagtctttctgataactgggagagggcagtaaggaggacttcctggagggggtgactgtccagagcctggaactgtgcccacaccagaagccatcagcagcaaggacaccatgcggcttccgggtgcgatgccagctctggccctcaaaggcgagctgctgttgctgtctctcctgttacttctggaaccacagatctctcagggcctggtcgtcacacccccggggccagagcttgtcctcaatgtctccagcaccttcgttctgacctgctcgggttcagctccggtggtgtgggaacggatgtcccaggagcccccacaggaaatggccaaggcccaggatggcaccttctccagcgtgctcacactgaccaacctcactgggctagacacgggagaatacttttgcacccacaatgactcccgtggactggagaccgatgagcggaaacggctctacatctttgtgccagatcccaccgtgggcttcctccctaatgatgccgaggaactattcatctttctcacggaaataactgagatcaccattccatgccgagtaacagacccacagctggtggtgacactgcacgagaagaaaggggacgttgcactgcctgtcccctatgatcaccaacgtggcttttctggtatctttgaggacagaagctacatctgcaaaaccaccattggggacagggaggtggattctgatgcctactatgtctacagactccaggtgtcatccatcaacgtctctgtgaacgcagtgcagactgtggtccgccagggtgagaacatcaccctcatgtgcattgtgatcgggaatgatgtggtcaacttcgagtggacatacccccgcaaagaaagtgggcggctggtggagccggtgactgacttcctcttggatatgccttaccacatccgctccatcctgcacatccccagtgccgagttagaagactcggggacctacacctgcaatgtgacggagagtgtgaatgaccatcaggatgaaaaggccatcaacatcaccgtggttgagagcggctacgtgcggctcctgggagaggtgggcacactacaatttgctgagctgcatcggagccggacactgcaggtagtgttcgaggcctacccaccgcccactgtcctgtggttcaaagacaaccgcaccctgggcgactccagcgctggcgaaatcgccctgtccacgcgcaacgtgtcggagacccggtatgtgtcagagctgacactggttcgcgtgaaggtggcagaggctggccactacaccatgcgggccttccatgaggatgctgaggtccagctctccttccagctacagatcaatgtccctgtccgagtgctggagctaagtgagagccaccctgacagtggggaacagacagtccgctgtcgtggccggggcatgccgcagccgaacatcatctggtctgcctgcagagacctcaaaaggtgtccacgtgagctgccgcccacgctgctggggaacagttccgaagaggagagccagctggagactaacgtgacgtactgggaggaggagcaggagtttgaggtggtgagcacactgcgtctgcagcacgtggatcggccactgtcggtgcgctgcacgctgcgcaacgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttacgagatccgatggaaggtgattgagtctgtgagctctgacggccatgagtacatctacgtggaccccatgcagctgccctatgactccacgtgggagctgccgcgggaccagcttgtgctgggacgcaccctcggctctggggcctttgggcaggtggtggaggccacagctcatggtctgagccattctcaggccacgatgaaagtggccgtcaagatgcttaaatccacagcccgcagcagtgagaagcaagcccttatgtcggagctgaagatcatgagtcaccttgggccccacctgaacgtggtcaacctgttgggggcctgcaccaaaggaggacccatctatatcatcactgagtactgccgctacggagacctggtggactacctgcaccgcaacaaacacaccttcctgcagcaccactccgacaagcgccgcccgcccagcgcggagctctacagcaatgctctgcccgttgggctccccctgcccagccatgtgtccttgaccggggagagcgacggtggctacatggacatgagcaaggacgagtcggtggactatgtgcccatgctggacatgaaaggagacgtcaaatatgcagacatcgagtcctccaactacatggccccttacgataactacgttccctctgcccctgagaggacctgccgagcaactttgatcaacgagtctccagtgctaagctacatggacctcgtgggcttcagctaccaggtggccaatggcatggagtttctggcctccaagaactgcgtccacagagacctggcggctaggaacgtgctcatctgtgaaggcaagctggtcaagatctgtgactttggcctggctcgagacatcatgcgggactcgaattacatctccaaaggcagcacctttttgcctttaaagtggatggctccggagagcatcttcaacagcctctacaccaccctgagcgacgtgtggtccttcgggatcctgctctgggagatcttcaccttgggtggcaccccttacccagagctgcccatgaacgagcagttctacaatgccatcaaacggggttaccgcatggcccagcctgcccatgcctccgacgagatctatgagatcatgcagaagtgctgggaagagaagtttgagattcggccccccttctcccagctggtgctgcttctcgagagactgttgggcgaaggttacaaaaagaagtaccagcaggtggatgaggagtttctgaggagtgaccacccagccatccttcggtcccaggcccgcttgcctgggttccatggcctccgatctcccctggacaccagctccgtcctctatactgccgtgcagcccaatgagggtgacaacgactatatcatccccctgcctgaccccaaacctgaggttgctgacgagggcccactggagggttcccccagcctagccagctccaccctgaatgaagtcaacacctcctcaaccatctcctgtgacagccccctggagccccaggacgaaccagagccagagccccagcttgagctccaggtggagccggagccggagctggaacagttgccggattcggggtgccctgcgcctcgggcggaagcagaggatagcttcctgtagggggctggcccctaccctgccctgcctgaagctcccccgctgccagcacccagcatctcctggcctggcctggccgggcttcctgtcagccaggctgcccttatcagctgtccccttctggaagctttctgctcctgacgtgttgtgccccaaaccctggggctggcttaggaggcaagaaaactgcaggggccgtgaccagccctctgcctccagggaggccaactgactctgagccagggttcccccagggaactcagttttcccatatgtaagatgggaaagttaggcttgatgacccagaatctaggattctctccctggctgacaggtggggagaccgaatccctccctgggaagattcttggagttactgaggtggtaaattaacttttttctgttcagccagctacccctcaaggaatcatagctctctcctcgcacttttatccacccaggagctagggaagagaccctagcctccctggctgctggctgagctagggcctagccttgagcagtgttgcctcatccagaagaaagccagtctcctccctatgatgccagtccctgcgttccctggcccgagctggtctggggccattaggcagcctaattaatgctggaggctgagccaagtacaggacacccccagcctgcagcccttgcccagggcacttggagcacacgcagccatagcaagtgcctgtgtccctgtccttcaggcccatcagtcctggggctttttctttatcaccctcagtcttaatccatccaccagagtctagaaggccagacgggccccgcatctgtgatgagaatgtaaatgtgccagtgtggagtggccacgtgtgtgtgccagatatggccctggctctgcattggacctgctatgaggctttggaggaatccctcaccctctctgggcctcagtttccccttcaaaaaatgaataagtcggacttattaactctgagtgccttgccagcactaacattctagagtatccaggtggttgcacatttgtccagatgaagcaaggccatataccctaaacttccatcctgggggtcagctgggctcctgggagattccagatcacacatcacactctggggactcaggaaccatgccccttccccaggcccccagcaagtctcaagaacacagctgcacaggccttgacttagagtgacagccggtgtcctggaaagcccccagcagctgccccagggacatgggaagaccacgggacctctttcactacccacgatgacctccgggggtatcctgggcaaaagggacaaagagggcaaatgagatcacctcctgcagcccaccactccagcacctgtgccgaggtctgcgtcgaagacagaatggacagtgaggacagttatgtcttgtaaaagacaagaagcttcagatgggtaccccaagaaggatgtgagaggtgggcgctttggaggtttgcccctcacccaccagctgccccatccctgaggcagcgctccatgggggtatggttttgtcactgcccagacctagcagtgacatctcattgtccccagcccagtgggcattggaggtgccaggggagtcagggttgtagccaagacgcccccgcacggggagggttgggaagggggtgcaggaagctcaacccctctgggcaccaaccctgcattgcaggttggcaccttacttccctgggatcccagagttggtccaaggagggagagtgggttctcaatacggtaccaaagatataatcacctaggtttacaaatatttttaggactcacgttaactcacatttatacagcagaaatgctattttgtatgctgttaagtttttctatctgtgtacttttttttaagggaaagattttaatattaaacctggtgcttctcactcac

SEQ ID NO: 119 - protein, 1106 aa, Homo sapiens [sequence omitted]

SEQ ID NO: 120 - DNA, 4795 bp, Homo sapiens
  source 1..4795: mol_type = genomic DNA
  CDS 20..4111
ccacgcgcagcggccggagatgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctgaactgcaccgtgtgggctgagtttaactcaggtgtcacctttgactgggactacccagggaagcaggcagagcggggtaagtgggtgcccgagcgacgctcccagcagacccacacagaactctccagcatcctgaccatccacaacgtcagccagcacgacctgggctcgtatgtgtgcaaggccaacaacggcatccagcgatttcgggagagcaccgaggtcattgtgcatgaaaatcccttcatcagcgtcgagtggctcaaaggacccatcctggaggccacggcaggagacgagctggtgaagctgcccgtgaagctggcagcgtaccccccgcccgagttccagtggtacaaggatggaaaggcactgtccgggcgccacagtccacatgccctggtgctcaaggaggtgacagaggccagcacaggcacctacaccctcgccctgtggaactccgctgctggcctgaggcgcaacatcagcctggagctggtggtgaatgtgcccccccagatacatgagaaggaggcctcctcccccagcatctactcgcgtcacagccgccaggccctcacctgcacggcctacggggtgcccctgcctctcagcatccagtggcactggcggccctggacaccctgcaagatgtttgcccagcgtagtctccggcggcggcagcagcaagacctcatgccacagtgccgtgactggagggcggtgaccacgcaggatgccgtgaaccccatcgagagcctggacacctggaccgagtttgtggagggaaagaataagactgtgagcaagctggtgatccagaatgccaacgtgtctgccatgtacaagtgtgtggtctccaacaaggtgggccaggatgagcggctcatctacttctatgtgaccaccatccccgacggcttcaccatcgaatccaagccatccgaggagctactagagggccagccggtgctcctgagctgccaagccgacagctacaagtacgagcatctgcgctggtaccgcctcaacctgtccacgctgcacgatgcgcacgggaacccgcttctgctcgactgcaagaacgtgcatctgttcgccacccctctggccgccagcctggaggaggtggcacctggggcgcgccacgccacgctcagcctgagtatcccccgcgtcgcgcccgagcacgagggccactatgtgtgcgaagtgcaagaccggcgcagccatgacaagcactgccacaagaagtacctgtcggtgcaggccctggaagcccctcggctcacgcagaacttgaccgacctcctggtgaacgtgagcgactcgctggagatgcagtgcttggtggccggagcgcacgcgcccagcatcgtgtggtacaaagacgagaggctgctggaggaaaagtctggagtcgacttggcggactccaaccagaagctgagcatccagcgcgtgcgcgaggaggatgcgggacgctatctgtgcagcgtgtgcaacgccaagggctgcgtcaactcctccgccagcgtggccgtggaaggctccgaggataagggcagcatggagatcgtgatccttgtcggtaccggcgtcatcgctgtcttcttctgggtcctcctcctcctcatcttctgtaacatgaggaggccggcccacgcagacatcaagacgggctacctgtccatcatcatggaccccggggaggtgcctctggaggagcaatgcgaatacctgtcctacgatgccagccagtgggaattcccccgagagcggctgcacctggggagagtgctcggctacggcgccttcgggaaggtggtggaagcctccgctttcggcatccacaagggcagcagctgtgacaccgtggccgtgaaaatgctgaaagagggcgccacggccagcgagcaccgcgcgctgatgtcggagctcaagatcctcattcacatcggcaaccacctcaacgtggtcaacctcctcggggcgtgcaccaagccgcagggccccctcatggtgatcgtggagttctgcaagtacggcaacctctccaacttcctgcgcgccaagcgggacgccttcagcccctgcgcggagaagtctcccgagcagcgcggacgcttccgcgccatggtggagctcgccaggctggatcggaggcggccggggagcagcgacagggtcctcttcgcgcggttctcgaagaccgagggcggagcgaggcgggcttctccagaccaagaagctgaggacctgtggctgagcccgctgaccatggaagatcttgtctgctacagcttccaggtggccagagggatggagttcctggcttcccgaaagtgcatccacagagacctggctgctcggaacattctgctgtcggaaagcgacgtggtgaagatctgtgactttggccttgcccgggacatctacaaagaccctgactacgtccgcaagggcagtgcccggctgcccctgaagtggatggcccctgaaagcatcttcgacaaggtgtacaccacgcagagtgacgtgtggtcctttggggtgcttctctgggagatcttctctctgggggcctccccgtaccctggggtgcagatcaatgaggagttctgccagcggctgagagacggcacaaggatgagggccccggagctggccactcccgccatacgccgcatcatgctgaactgctggtccggagaccccaaggcgagacctgcattctcggagctggtggagatcctgggggacctgctccagggcaggggcctgcaagaggaagaggaggtctgcatggccccgcgcagctctcagagctcagaagagggcagcttctcgcaggtgtccaccatggccctacacatcgcccaggctgacgctgaggacagcccgccaagcctgcagcgccacagcctggccgccaggtattacaactgggtgtcctttcccgggtgcctggccagaggggctgagacccgtggttcctccaggatgaagacatttgaggaattccccatgaccccaacgacctacaaaggctctgtggacaaccagacagacagtgggatggtgctggcctcggaggagtttgagcagatagagagcaggcatagacaagaaagcggcttcagctgtaaaggacctggccagaatgtggctgtgaccagggcacaccctgactcccaagggaggcggcggcggcctgagcggggggcccgaggaggccaggtgttttacaacagcgagtatggggagctgtcggagccaagcgaggaggaccactgctccccgtctgcccgcgtgactttcttcacagacaacagctactaagcagcatcggacaagacccccagcacttgggggttcaggcccggcagggcgggcagagggctggaggcccaggctgggaactcatctggttgaactctggtggcacaggagtgtcctcttccctctctgcagacttcccagctaggaagagcaggactccaggcccaaggctcccggaattccgtcaccacgactggccagggcacgctccagctgccccggcccctccccctgagattcagatgtcatttagttcagcatccgcaggtgctggtcccggggccagcacttccatgggaatgtctctttggcgacctcctttcatcacactgggtggtggcctggtccctgttttcccacgaggaatctgtgggtctgggagtcacacagtgttggaggttaaggcatacgagagcagaggtctcccaaacgccctttcctcctcaggcacacagctactctccccacgagggctggctggcctcacccacccctgcacagttgaagggaggggctgtgtttccatctcaaagaaggcatttgcagggtcctcttctgggcctgaccaaacagccaactagcccctggggtggccaccagtatgacagtattatacgctggcaacacagaggcagcccgcacacctgcgcctgggtgttgagagccatcctgcaagtctttttc

SEQ ID NO: 121 - protein, 1363 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFSCKGPGQNVAVTRAHPDSQGRRRRPERGARGGQVFYNSEYGELSEPSEEDHCSPSARVTFFTDNSY

SEQ ID NO: 122 - DNA, 33 bp, synthetic construct
  misc_feature 1..33: note = Synthetic primer
  source 1..33: mol_type = other DNA
tacttggcagtacatctacgtattagtcatcgc

SEQ ID NO: 123 - DNA, 39 bp, synthetic construct
  misc_feature 1..39: note = Synthetic primer
  source 1..39: mol_type = other DNA
cggagatctgtagtcttgcacgtacacgtaggagctggc

SEQ ID NO: 124 - DNA, 1752 bp, Homo sapiens
  source 1..1752: mol_type = genomic DNA
atgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgtacgtgcaagactacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaagatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 125 - protein, 583 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 126 - DNA, 81 bp, Homo sapiens
  source 1..81: mol_type = genomic DNA
tacaattgaggacaagcgtatgtccacgaagtagtttaactggacgaggcgtgcttatttgcacatcataaatcctatacc

SEQ ID NO: 127 - DNA, 1752 bp, Homo sapiens; identical to SEQ ID NO: 124
  source 1..1752: mol_type = genomic DNA

SEQ ID NO: 128 - protein, 583 aa, Homo sapiens; identical to SEQ ID NO: 125

SEQ ID NO: 129 - DNA, 25 bp, synthetic construct
  misc_feature 1..25: note = Synthetic primer
  source 1..25: mol_type = other DNA
ctgagagggatcgataattacaaga

SEQ ID NO: 130 - DNA, 24 bp, synthetic construct
  misc_feature 1..24: note = Synthetic primer
  source 1..24: mol_type = other DNA
attggcactcaaatttgttttcag

SEQ ID NO: 131 - DNA, 24 bp, synthetic construct
  misc_feature 1..24: note = Synthetic primer
  source 1..24: mol_type = other DNA
atgtttgtaccagacgtccttacg

SEQ ID NO: 132 - DNA, 17 bp, synthetic construct
  misc_feature 1..17: note = Synthetic primer
  source 1..17: mol_type = other DNA
tgcgaagcaccctggaa

SEQ ID NO: 133 - DNA, 25 bp, synthetic construct
  misc_feature 1..25: note = Synthetic primer
  source 1..25: mol_type = other DNA
cccaacttccaatgctctcctaatg

SEQ ID NO: 134 - DNA, 25 bp, synthetic construct
  misc_feature 1..25: note = Synthetic primer
  source 1..25: mol_type = other DNA
gcacactaggtttgccgagtagacc

SEQ ID NO: 135 - DNA, 20 bp, synthetic construct
  misc_feature 1..20: note = Synthetic primer
  source 1..20: mol_type = other DNA
taagggttacttgggttgcc

SEQ ID NO: 136 - DNA, 19 bp, synthetic construct
  misc_feature 1..19: note = Synthetic primer
  source 1..19: mol_type = other DNA
tatccagagggtcttcagc

SEQ ID NO: 137 - DNA, 24 bp, synthetic construct
  misc_feature 1..24: note = Synthetic primer
  source 1..24: mol_type = other DNA
ctgtgcctcagcctcttctcattc

SEQ ID NO: 138 - DNA, 24 bp, synthetic construct
  misc_feature 1..24: note = Synthetic primer
  source 1..24: mol_type = other DNA
ttgggaacttctcctccttgttgg

SEQ ID NO: 139 - DNA, 19 bp, synthetic construct
  misc_feature 1..19: note = Synthetic primer
  source 1..19: mol_type = other DNA
gaggtgaacaacccacaga

SEQ ID NO: 140 - DNA, 20 bp, synthetic construct
  misc_feature 1..20: note = Synthetic primer
  source 1..20: mol_type = other DNA
tattggcacactctctaccc

SEQ ID NO: 141 - DNA, 18 bp, synthetic construct
  misc_feature 1..18: note = Synthetic primer
  source 1..18: mol_type = other DNA
ccctggacccaaggcagt

SEQ ID NO: 142 - DNA, 20 bp, synthetic construct
  misc_feature 1..20: note = Synthetic primer
  source 1..20: mol_type = other DNA
aggcttagagtgcttccggg

SEQ ID NO: 143 - DNA, 21 bp, synthetic construct
  misc_feature 1..21: note = Synthetic primer
  source 1..21: mol_type = other DNA
gcttgtttgctgtgcggagac

SEQ ID NO: 144 - DNA, 21 bp, synthetic construct
  misc_feature 1..21: note = Synthetic primer
  source 1..21: mol_type = other DNA
gtttctgaagtaggcgaacat

(19) United States
(12) Patent Application Publication
(10) Pub. No.: US 2024/0368248 A1
(43) Pub. Date: Nov. 7, 2024

(54) GROWTH FACTOR ANTAGONISTS FOR ORGAN TRANSPLANT ALLOIMMUNITY AND ARTERIOSCLEROSIS

(71) Applicant: VEGENICS PTY LIMITED, South Yarra (AU)

(72) Inventors: Kari Alitalo, Helsinki (FI); Karl B. Lemström, Helsinki (FI); Antti I. Nykänen, Helsinki (FI)

(21) Appl. No.: 18/775,969

(22) Filed: Jul. 17, 2024

Related U.S. Application Data
(63) Continuation of application No. 17/153,636, filed on Jan. 20, 2021, now abandoned.
Application No. 17/153,636 is a continuation of application No. 15/820,212, filed on Nov. 21, 2017, now abandoned.
Application No. 15/820,212 is a division of application No. 14/687,556, filed on Apr. 15, 2015, now Pat. No. 9,896,499.
Application No. 14/687,556 is a division of application No. 12/024,454, filed on Feb. 1, 2008, now Pat. No. 9,073,997.
(60) Provisional application No. 60/888,067, filed on Feb. 2, 2007.
(60) Provisional application No. 60/888,305, filed on Feb. 5, 2007.

(51) Int. Cl. C07K 14/71; A61K 39/00; A61K 39/395; A61K 45/06; C07K 16/22; C07K 16/28; C12N 15/113

(52) U.S. Cl. CPC C07K 14/71; A61K 39/3955; A61K 45/06; C07K 16/22; C07K 16/2863; C12N 15/1136; C12N 15/1138; A61K 2039/505; C07K 2317/24; C07K 2317/73; C07K 2317/76; C07K 2319/30; C12N 2310/11; C12N 2320/30

(57) ABSTRACT

The present invention provides materials and methods for antagonizing the function of vascular endothelial growth factor receptors, platelet derived growth factor receptors and other receptors, to prevent, inhibit, or ameliorate allograft rejection or arteriosclerosis in organisms that receive an organ transplant.

### CROSS-REFERENCE TO RELATED APPLICATIONS

The present application is a continuation of U.S. patent application Ser. No. 17/153,636, filed on Jan. 20, 2021, which is a continuation of U.S. patent application Ser. No. 15/820,212, filed on Nov. 21, 2017; which is a divisional of U.S. patent application Ser. No. 14/687,556, filed on Apr. 15, 2015, now U.S. Pat. No. 9,896,499; which is a divisional of U.S. patent application Ser. No. 12/024,454, filed on Feb. 1, 2008, now U.S. Pat. No. 9,073,997; which claims priority benefit under § 119 of U.S. Provisional Application No. 60/888,067, filed on Feb. 2, 2007, and U.S. Provisional Application No. 60/888,305, filed on Feb. 5, 2007. The disclosure of each priority application is incorporated herein by reference in its entirety.

### INCORPORATION BY REFERENCE OF MATERIAL SUBMITTED ELECTRONICALLY

This application contains, as a separate part of disclosure, a sequence listing in computer-readable form (filename: 42606F_Seqlisting.xml; 326,220 bytes; created Jul. 16, 2024) which is incorporated by reference in its entirety.

### BACKGROUND

Interaction of innate and adaptive immunity leads to alloimmune responses that may be detrimental to cardiac allografts and heart transplant recipients. Antigen-presenting cells (APC) initiate allorecognition by processing foreign peptides, migrating to secondary lymphoid tissue, and presenting these peptides to recipient lymphocytes. After recognition, alloreactive T lymphocytes proliferate and migrate to their target tissue. Although the current immunosuppressive regiments effectively inhibit the proliferation of alloreactive T lymphocytes, they have several metabolic, infectious, renal and malignant side-effects. In addition, the long-term survival of heart transplant patients is decreased by gradual concentric intimal thickening of large and small allograft coronary arteries—cardiac allograft arteriosclerosis—despite the use of modern immunosuppression.

The lymphatic network forms a conduct system that transfers interstitial fluids and inflammatory cells from the target tissue to secondary LN, and is essential in the activation of adaptive immunity. Vascular endothelial growth factor C (VEGF-C) and its receptor VEGFR-3 are the key regulators for lymphatic growth. VEGF-C is essential in the development and maintenance of the lymphatic system, and improper lymphangiogenesis is related to many pathological conditions. Lymphatic vascular insufficiency leads to lymphedema, whereas extensive lymphangiogenesis is often seen in tumor metastasis and inflammatory situations. During inflammation, macrophages are a rich source for VEGF-C, and pro-inflammatory cytokines such as TNF-α, IL-1α and -β[(15) as well as TGF-β (16)] induce VEGF-C expression. Dendritic cells (DC) may express VEGFR-3 during inflammation (Hamrah et al., (2003), Am J Pathol., 163: 57-6817) which renders them responsive for VEGF-C-induced migration (Chen et al., (2004), Nat. Med., 10: 813-81518). Also, lymphatic endothelial cells (EC)—in contrast to vascular EC—secrete CCL21 chemokine that mediates CCR7+ inflammatory cell traffic to lymphoid organs and peripheral effector sites. (See Kriehuber et al., (2001), J. Exp. Med., 194: 797-808; Saeki et al., (1999), J. Immunol. 162: 2472-2475; Campbell et al., (1998), J. Cell. Biol. 141: 1053-1059; and Lo et al., (2003), J. Clin. Invest. 112: 1495-1505.

Corneal transplant is currently the most successful tissue transplantation procedures in humans, with a first year survival rate as high as 90%, even in the absence of routine HLA tying and with minimal immunosuppressive therapy. The healthy cornea is generally a non-vascular tissue. DeVries, U.S. Patent Publication No. 2003/0180294 purports to describe use of a VEGFR-3 inhibitor to reduce lymhangiogenesis in a transplanted cornea to extend its survival. Chen et al., (2004), Nat. Med., 10: 813-81518, purport to describe that blockade of VEGFR-3 in corneal transplants suppresses corneal antigen presenting dendritic cells, delaying rejection of corneal transplants. The same research group previously reported that dendritic cells in the cornea were VEGFR-3⁺, whereas similar dendritic cells were absent in the skin, even though the cornea shares embryological origins with the skin.

A need exists for all transplanted tissues and organs, especially vascularized tissues and organs, for new materials and methods for slowing, reducing, or eliminating rejection and also for slowing, reducing, or eliminating graft arteriosclerosis.

### SUMMARY OF THE INVENTION

The present invention provides materials and methods to improve the outcomes of transplant recipients, e.g., by postponing or inhibiting or reducing or ameliorating an immune reaction against the transplant (rejection), and/or by postponing or inhibiting or reducing or ameliorating arteriosclerosis or other deleterious side effects often associated with transplants.

Thus, in one embodiment, the invention is a method for inducing tolerance or inhibiting rejection of a cell, tissue, or organ transplant, or for inhibiting arteriosclerosis in a transplant recipient. For example, one such method comprises administering to a mammalian transplant recipient a composition that comprises a growth factor inhibitor, such as an endothelial growth factor inhibitor, in an amount effective to induce tolerance for the transplant by the recipient, or inhibit rejection, or inhibit arteriosclerosis. “Transplant recipient” refers to the mammalian subject or patient that receives the transplanted cells, tissue, or organ, from a donor. Preferred mammalian donors and recipients are human donors and recipients. The method also may be practiced with pets (dogs, cats), racing animals (dogs, horses); agriculturally important animals (cows, pigs); non-human primates (chimps, gorillas, etc.); and important lab animals (e.g., rodents). The method may be practiced with xenografts of from one donor species to a different recipient species.

In a related embodiment, the invention is a method for inducing tolerance or inhibiting rejection of a cell, tissue, or organ transplant, or for inhibiting arteriosclerosis in a transplant recipient, comprising: administering to a mammalian transplant recipient a composition that comprises an nucleic acid that comprises a nucleotide sequence that encodes a growth factor inhibitor, such as an endothelial growth factor inhibitor, wherein the nucleic acid is expressible in cells of the recipient or expressible in the transplanted cell, tissue, or organ to produce an amount of the endothelial growth factor inhibitor effective to induce tolerance for the transplant by the recipient, or inhibit rejection, or inhibit arteriosclerosis. To facilitate expression of the encoded inhibitor, the nucleic acid preferably comprises at least one expression control sequence operatively connected to the sequence that encodes the endothelial growth factor inhibitor. Exemplary expression control sequences include promoters and enhancers, for example. In some preferred variations, the method comprises administering an expression vector that comprises the nucleic acid to the transplant recipient. For example, the vector comprises a replication deficient viral vector; such as a retrovirus, an adenovirus, an adeno-associated virus, a vaccinia virus or a herpesvirus vector. In some variations, the vector is inducible by administration of an exogenous pharmaceutical agent. In other variations, expression of the vector is induced by an endogenous stress in the organ transplant recipient, such as an elevation of a biological marker correlated with rejection. In still other variations, the vector is constitutively expressed.

In still related embodiments, the method is directed to a method for reducing a transplant recipients dependence or need for immunosuppressive drugs, by administering such inhibitors to the recipient, in an amount effective to reduce the dose or doing of one or more immunosuppressant drugs administered to the transplant recipient.

For all embodiments of the invention, whether therapeutic agent is a polypeptide, an antibody, a polynucleotide, a small molecule, or some combination thereof, the administered composition preferably further includes a pharmaceutically acceptable carrier. The composition may include one inhibitor or may include a combination of inhibitors, or may include one inhibitor that targets multiple growth factor or growth factor receptor targets described herein.

Methods of the invention can be practiced with respect to all variations of transplanted cells or tissue. For example, in some variations, the transplant is a xenograft, and the method induces tolerance for the xenograft or inhibits xenograft rejection, or reduces xenograft-related arteriosclerosis. In other preferred variations, the transplant is an allograft transplant, and the composition is administered in an amount effective to induce tolerance for the allograft or inhibit alloimmunity, or reduce graft related arteriosclerosis.

In some variations, the transplant may be limited to specified cell types or to a tissue transplant. For example, the cell or tissue comprises embryonic stem cells, pluripotent stem cells, hematopoietic precursor cells, neuronal precursor cells, or endothelial precursor cells. In some variations, the cell or tissue comprises a member selected from the group consisting of pancreatic islet cells, cardiac myocytes, bone marrow cells, endothelial cells, and skin cells.

In some variations of the invention, treatment of corneal transplant patients is specifically excluded from the invention.

In many preferred embodiments, the transplant is an organ or organ fragment capable of performing functions of the organ or capable of regenerating into the organ. For example, the method is practiced on a recipient of at least one transplanted organ, or fragment thereof, selected from the group consisting of a heart, a kidney, a lung, a liver, an intestine, a pancreas, skin, and bone. In some highly preferred variations, the method is practiced on the recipient of at least one transplanted organ selected from the group consisting of heart, lung, liver, and kidney. Treatment of cardiac (heart) transplant recipients is highly preferred.

All variety of formulations and routes of administration are contemplated. For example, in some variations, the composition is administered locally to the transplanted cell, tissue, or organ in the recipient. In some variations, the composition is administered systemically to the recipient. In some variations of the method, the composition is administered intravenously, intramuscularly, or intraperitoneally, or perorally.

To provide just a few exemplary variations, pharmacological agents may preferably be administered systemically. Monoclonal antibodies may be administered intravenously, intramuscularly, or intraperitoneally. Receptor tyrosine kinase inhibitors may be administered perorally or intravenously. Nucleic acid or vectors preferably would be administered intravascularly or intraparenchymally to the transplanted organ, optionally during the organ procurement.

All variations of timing of administration also are contemplated.

For example, in some variations, the method further comprises administering the composition to the organ or the organ donor before the transplant. (In other variations, the inhibitor composition administered at this stage contains a different inhibitor from the composition administered to the recipient.) As described below in detail, donor cells are implicated in graft rejection, and administering the inhibitors to the donor organ or donor prior to the transplant is contemplated to have beneficial effects during the critical perioperative period.

In some variations, the method further comprises repeated administration of the composition to the recipient.

The composition may be administered to the recipient perioperatively, relative to the transplant operation. The composition may be administered for varying lengths of time after the transplant operation for prophylaxis, e.g., 1, 2, 3, 4, 5, 6, 7, 10, 14, 21, 28, 30, 31, 45, 56, 60, 90, 120, 180 days post-transplant. All durations from one day to fifty years post-transplant are specifically contemplated.

In other variations, the method is practiced at discrete times to ameliorate acute rejection events. For example, in some variations, the method is practiced upon detection of symptoms of rejection, and the inhibitor is administered in an amount effective to alleviate the symptoms. In some variations, the method comprises a step of screening the organ transplant recipient for symptoms of an acute rejection reaction; where the composition that contains the inhibitor is administered to the recipient upon detection of symptoms of acute rejection, in an amount effective to inhibit the rejection.

A wide variety of growth factor (including endothelial growth factor) inhibitors are described below in detail for practice of the invention. Some of the inhibitors bind to a growth factor or to a receptor, and may be described below as binding constructs. Other inhibitors may act indirectly, e.g., at the level of effecting gene or protein expression, or inhibiting downstream signaling by an activated receptor.

In some variations, the endothelial growth factor inhibitor comprises a compound that inhibits stimulation of at least one receptor selected from the group consisting of VEGFR-1, VEGFR-2, VEGFR-3, PDGFR-alpha, and PDGFR-beta by a growth factor ligand of said at least one receptor. In some highly preferred variations, the endothelial growth factor inhibitor comprises a compound that inhibits stimulation of VEGFR-3 by VEGF-C or inhibits stimulation of VEGFR-3 by VEGF-D.

For example, in some embodiments, the compound comprises an antibody substance selected from the group consisting of antibody substances that immunoreact with VEGFR-3, antibody substances that immunoreact with VEGF-C, and antibody substances that immunoreact with VEGF-D. The term antibody substance is intended to refer to traditional antibodies and also to the wide variety of engineered antibody fragments and variants that are engineered for therapeutic purposes. For example, exemplary preferred antibody substances include a humanized antibody, a human antibody, a monoclonal antibody, a fragment of an antibody that retains antigen binding characteristics, and a polypeptide that comprises an antigen binding fragment of an antibody. A preferred antibody substance is a monoclonal antibody (preferably humanized or fully human) that binds VEGFR-3 or VEGF-C or VEGF-D and inhibits binding between VEGFR-3 and VEGF-C or -D.

Yet another preferred class of inhibitor substances are soluble receptor constructs that are capable of binding circulating endothelial cell growth factor molecules and preventing them from binding and stimulating receptors expressed on endothelial or other cell surfaces. Thus, in some embodiments, the endothelial growth factor inhibitor comprises a soluble receptor that binds to at least one endothelial cell growth factor. In a preferred variation, the endothelial growth factor inhibitor comprises a soluble VEGFR-3 polypeptide that binds to VEGF-C or VEGF-D. For example, the soluble VEGFR-3 polypeptide comprises the VEGFR-3 extracellular domain, or a fragment thereof sufficient to bind VEGF-C or VEGF-D. Exemplary fragments include the first and second immunoglobulin-like domains of the VEGFR-3; or include the first, second, and third immunoglobulin-like domains of the VEGFR-3. In some preferred variations, the soluble receptor is fused to an immunoglobulin constant domain to increase serum half life. Such constructs can be expressed recombinantly, as fusion proteins.

In still another variation, the inhibitor comprises an antisense nucleic acid or an interfering RNA nucleic acid that inhibits expression of an endothelial cell growth factor or endothelial cell growth factor receptor. Preferred examples include a short interfering RNA that inhibits expression of a protein selected from the group consisting of VEGFR-3, VEGF-C, and VEGF-D; and an antisense nucleic acid that inhibits expression of a protein selected from the group consisting of VEGFR-3, VEGF-C, and VEGF-D.

In still other variations of the invention, the inhibitor compound comprises bevacizumab (Avastin®) or Ranibizumab (Lucentis®), both marketed by Genentech.

In some variations of the invention, a composition that comprises two different inhibitors is administered; or a two or more inhibitor compositions are administered. Combinations that include an inhibitor of VEGFR-3 (or VEGF-C or VEGF-D) in combination with an inhibitor of one or more of the following growth factor receptors (or their ligands) are particularly preferred: VEGFR-1, VEGFR-2, PDGFR-alpha, and PDGFR-beta.

In still other variations of the invention, the compound is a multivalent inhibitor of two or more receptors selected from the group consisting of VEGFR-1, VEGFR-2, VEGFR-3, PDGFR-alpha, and PDGFR-beta. For example, in some variations, the method of the invention comprises administering to the transplant recipient a composition that inhibits ligand binding to VEGFR-2 and inhibits ligand binding to VEGFR-3.

It is contemplated that the inhibitors of the invention protect the transplant recipient by different mechanisms than traditional or existing immunosuppressive regimens. In some variations of the invention, the inhibitors of the invention are co-administered with immunosuppressive therapy. The combination is expected to provide at least additive, and preferably synergistic, effects compared to either type of agent alone. The synergistic effects can be in the form of increased efficacy for survival of the transplant; and also for reduced side effects, possibly due to the need for reduced dosing of the immunosuppressive agents.

Thus, in some variations, the method of the invention further comprises administering an immunosuppressive agent to the organ transplant recipient. Exemplary classes of immunosuppressive agents include corticosteriods, calcineurine inhibitors, antiproliferative agents, monoclonal antilymphocyte antibodies, and polyclonal antilymphocyte antibodies. Exemplary immunosuppressive agents include Tacrolimus, Mycophenolic acid, Prednisone, Ciclosporin, Azathioprine, Basiliximab, Daclizumab, Muromonab-CD3, Mycophenolate Mofetil, Sirolimus, Methylprednisolone, Thymoglobulin, OKT3, Rapamycin, Azathioprine, Cyclosporine, and Interleukin-2 Receptor Antagonist. These agents can be administered singly or in combination.

In yet another variation, the method of the invention further comprising administering an antibiotic or antifungal agent to the recipient, to protect the recipient from infections.

In still further variations of the invention, the transplant recipient is helped by pre-treating the donor (or the tissue or organ to be transplanted), with the inhibitory agent. Thus, in some embodiments, the method of the invention further comprises administering to a donor organism a composition that comprises an endothelial growth factor inhibitor, prior to harvesting a cell, tissue, or organ for transplantation into the recipient. In other embodiments, the method further comprises contacting a cell, tissue, or organ with a composition that comprises an endothelial growth factor inhibitor, prior to transplanting the cell, tissue, or organ into the mammalian organ transplant recipient. In still other embodiments, the method further comprises administering to a donor organism, prior to harvesting cells, tissue, or an organ for transplantation, a composition that comprises an nucleic acid that comprises a nucleotide sequence that encodes an endothelial growth factor inhibitor, wherein the nucleic acid is expressible in cells of the tissue or organ to be transplanted. In still further variations, the method further comprises contacting a cell, tissue, or organ with a composition that comprises an nucleic acid that comprises a nucleotide sequence that encodes an endothelial growth factor inhibitor, prior to transplanting the cell, tissue, or organ into the recipient.

Other embodiments of the invention do not require administration of a growth factor inhibitor to the recipient at all. For example, one embodiment of the invention is a method of preparing a donor cell, tissue, or organ for allograft or xenograft transplantation comprising contacting the cell, tissue, or organ with a composition that comprises a growth factor inhibitor, such as an endothelial growth factor inhibitor, prior to transplanting the cell, tissue, or organ into a mammalian organ transplant recipient. A related embodiment is a method of preparing a donor cell, tissue, or organ for allograft or xenograft transplantation comprising contacting the cell, tissue, or organ with a composition that comprises an nucleic acid that comprises a nucleotide sequence that encodes a growth factor inhibitor, such as an endothelial growth factor inhibitor, prior to transplanting the cell, tissue, or organ into a mammalian organ transplant recipient.

Other variations of the invention are directed to material, useful for practicing methods of the invention. For example, in one embodiment, the invention is a composition that comprises an endothelial growth factor inhibitor, an immunosuppressant, and a pharmaceutically acceptable carrier. Preferably, the inhibitor and the immunosuppressant are present in the composition in synergistically effective amounts.

In a related variation, the invention is a kit or unit dose in which the inhibitor and the immunosuppressant are packaged together, but not in admixture.

The present invention relates to compositions and methods of use thereof for the inhibition of graft (e.g., allograft) rejection and graft-related arteriosclerosis, and inhibition of other effects of members of the PDGF/VEGF family of growth factors: VEGF-A, VEGF-B, VEGF-C, VEGF-D, VEGF-E, PIGF, PDGF-A, PDGF-B, PDGF-C, and PDGF-D, each of which is able to bind at least one growth factor receptor tyrosine kinase and stimulate phosphorylation of the same. The compositions of the invention include binding constructs that bind one or more PDGF/VEGF molecules. The binding constructs include one or more binding units. Likewise, many of the inhibitors for use in practicing the invention are described herein as binding units or binding constructs.

In some embodiments, the binding unit comprises a polypeptide, e.g., a fragment of a growth factor receptor tyrosine kinase extracellular domain. The invention also provides nucleic acids encoding such binding constructs, and uses thereof. Binding units are not limited to receptor fragments, nor are they limited to polypeptides, but rather comprise any species that binds a growth factor or binds a receptor, and thereby inhibits the circulating growth factor from binding or stimulating the receptor naturally expressed on the surface of cells. Administration of the compositions of the invention to patients inhibits growth factor stimulation of VEGF receptors and/or PDGF receptors (e.g., inhibits phosphorylation of the receptors) and thereby inhibits biological responses mediated through the receptors including, but not limited to, PDGFR—and/or VEGFR-mediated angiogenesis and lymphangiogenesis.

Each member of the growth factor genus described above binds with high affinity to, and stimulation phosphorylation of, at least one PDGF receptor or VEGF receptor (or receptor heterodimer) selected from VEGFR-1, VEGFR-2, VEGFR-3, PDGFR-alpha, and PDGFR-beta. This statement refers to well known properties of the growth factors toward their cognate receptors, and is not meant as a limiting feature per se of the binding constructs of the invention. (For example, VEGF-A has been shown to bind to VEGFR-1 and VEGFR-2 and induce tyrosine phosphorylation of both receptors and initiate downstream receptor signaling.) However, preferred binding units of the invention do more than simply bind their target growth factors: a preferred binding construct also inhibits the growth factor(s) to which it binds from stimulating phosphorylation of at least one (and preferably all) of the receptor tyrosine kinases to which the growth factor(s) bind. Stimulation of tyrosine phosphorylation is readily measured using in vitro cell-based assays and anti-phosphotyrosine antibodies. Because phosphorylation of the receptor tyrosine kinases is an initial step in a signaling cascade, it is a convenient indicator of whether the binding construct is capable of inhibiting growth factor-mediated signal transduction that leads to cell migration, cell growth, and other responses. A number of other cell based and in vivo assays can be used to confirm the growth factor neutralizing properties of binding constructs of the invention.

As described herein, binding constructs can be chemically modified (e.g., heterologous peptide fusions, glycosylation, pegylation, etc.) to impart desired characteristics, while maintaining their specific growth factor binding properties. An exemplary peptide fusion comprises a immunoglobulin constant domain fragment. Exemplary desired characteristics imparted by chemical modifications include increased serum half life, increased solubility in an aqueous medium, and the ability to target a specific cell population, e.g., cancer cells.

Binding constructs and units that are “specific” for a particular growth factor are binding constructs and units that specifically recognize a circulating, active form of the growth factor. Preferably, the binding constructs specifically bind other forms of the growth factors as well. By way of example, VEGF-A exists in multiple isoforms, some of which circulate and others of which associate with heparin sulfate proteoglycans on cell surfaces. Binding constructs that are specific for VEGF-A bind to at least a circulating isoform, preferably all circulating isoforms, and more preferably, bind other major isoforms as well. By way of another example, VEGF-C is translated as a prepro-molecule with extensive amino-terminal and carboxy-terminal propeptides that are cleaved to yield a “fully processed” form of VEGF-C that binds and stimulates VEGFR-2 and VEGFR-3. Binding constructs specific for VEGF-C bind to at least the fully processed form of VEGF-C, and preferably also bind to partly processed forms and unprocessed forms.

Additional description is used herein when a more specialized meaning is intended. For example, VEGF-B167 is heparin bound whereas VEGF-B186 is freely secreted. An binding construct of the invention that minimally binds the circulating isoform is said to be specific for VEGF-B, and such a binding construct preferably also binds the heparin bound form. A binding construct of the invention that is “specific for heparin-bound VEGF-B” or “specific for VEGF-B167” is a binding construct that differentially recognizes the heparin bound isoform, compared to the freely circulating isoform. A binding construct of the invention that is specific for VEGF-B186” is a binding construct that differentially recognizes the circulating form, compared to the heparin bound form. Binding constructs specific for each isoform of a growth factor are contemplated as components of some embodiments of the binding constructs of the invention.

The designations “first” and “second” and “third” in respect to the binding units of the binding constructs is for ease and clarity in description only, and is not meant to signify a particular order, e.g., order in the amino acid sequence of a polypeptide binding construct.

A binding construct comprising two or more binding units may further comprise a linker connecting adjacent binding units. The linker may take on a number of different forms. Preferably, the linker comprises a peptide which allows adjacent binding units to be linked to form a single polypeptide.

The invention also includes compositions comprising a polypeptide, binding construct, or nucleic acid encoding the same, together with a pharmaceutically acceptable carrier. Such compositions may further comprise a pharmaceutically acceptable diluent, adjuvant, or carrier medium.

Nucleic acids (polynucleotides) of the invention include nucleic acids that constitute binding units, e.g., aptamers, and also nucleic acids that encode polypeptide binding units and constructs, which may be used for such applications as gene therapy and recombinant in vitro expression of polypeptide binding constructs. In some embodiments, nucleic acids are purified or isolated. In some embodiments, polynucleotides further comprise a promoter sequence operatively connected to a nucleotide sequence encoding a polypeptide, wherein the promoter sequence promotes transcription of the sequence that encodes the polypeptide in a host cell. Polynucleotides may also comprise a polyadenylation sequence. Other nucleic acids of the invention (e.g., antisense nucleic acids, interfering RNA nucleic acids) operate to inhibit transcription or translation of growth factor genes or receptor genes.

Vectors comprising polynucleotides are also aspects of the invention. Such vectors may comprise an expression control sequence operatively connected to the sequence that encodes the polypeptide, and the vector may be selected from the group consisting of a lentivirus vector, an adeno-associated viral vector, an adenoviral vector, a liposomal vector, and combinations thereof. In some embodiments, the vector comprises a replication-deficient adenovirus, said adenovirus comprising the polynucleotide operatively connected to a promoter and flanked by adenoviral polynucleotide sequences. Host cells comprising the polynucleotides, vectors and other nucleic acids, and methods for using the same to express and isolate the binding constructs and units are also aspects of the invention.

For binding units of a binding construct that comprises an aptamer, the aptamer may be generated by preparing a library of nucleic acids; contacting the library of nucleic acids with a growth factor, wherein nucleic acids having greater binding affinity for the growth factor (relative to other library nucleic acids) are selected and amplified to yield a mixture of nucleic acids enriched for nucleic acids with relatively higher affinity and specificity for binding to the growth factor. The processes may be repeated, and the selected nucleic acids mutated and rescreened, whereby a growth factor aptamer is be identified. Nucleic acids may be screened to select for molecules that bind to more than growth factor.

In one aspect of the invention, the binding construct comprises a purified polypeptide comprising an amino acid sequence at least 95% identical to a vascular endothelial growth factor receptor 3(VEGFR-3) fragment, wherein the VEGFR-3 fragment comprises an amino acid sequence consisting of a portion of SEQ ID NO: 6, wherein the carboxy-terminal residue of the fragment is selected from the group consisting of positions 211 to 247 of SEQ ID NO: 6. The fragment, and the polypeptide comprising the same, specifically bind to at least one growth factor selected from the group consisting of human vascular endothelial growth factor-C (VEGF-C), and human vascular endothelial growth factor-D (VEGF-D). In some embodiments the VEGFR-3 fragments has an amino terminal amino acid selected from the group consisting of positions 1 to 47 of SEQ ID NO: 6. In some embodiments, the polypeptide comprises an amino acid sequence selected from the group consisting of SEQ ID NOS: 36 and 38. In some embodiments, the fragment has an amino acid sequence selected from the group consisting of positions 1-226 and 1-229 of SEQ ID NO: 6. In some embodiments, the polypeptide is part of a binding construct, and the polypeptide is operatively connected with a second polypeptide that binds at least one growth factor selected from the group consisting of VEGF-A, VEGF-B, VEGF-C, VEGF-D, VEGF-E, PIGF, PDGF-A, PDGF-B, PDGF-C, and PDGF-D. In some embodiments, the second polypeptide is selected from the group consisting of a polypeptide comprising a vascular endothelial growth factor receptor extracellular domain fragment, a platelet derived growth factor receptor extracellular domain fragment, and a polypeptide comprising an antigen binding fragment of an antibody that immunoreacts with the at least one of said growth factors. In some embodiments, at least one of the polypeptides is encoded by a polynucleotide comprising a nucleotide sequence selected from the group consisting of SEQ ID NOS: 35 and 37.

In another aspect of the invention, a binding construct comprises a purified polypeptide comprising an amino acid sequence at least 95% identical to a VEGFR-2 fragment, wherein the VEGFR-2 fragment comprises an amino acid sequence consisting of a portion of SEQ ID NO: 4, wherein the amino terminal amino acid of the VEGFR-2 fragment is selected from the group consisting of positions 106-145 of SEQ ID NO: 4, wherein the carboxy terminal amino acid of the VEGFR-2 fragment is selected from the group consisting of positions 203 to 240 of SEQ ID NO: 4, and wherein the VEGFR-2 fragment and the polypeptide bind VEGF-C or VEGF-D. In some embodiments, the polypeptide comprises an amino acid sequence selected from the group consisting of SEQ ID NO: 22, 24, and 26. In some embodiments, the fragment consists of an amino acid sequence selected from the group consisting of residues 118-220, 118-226, and 118-232 of SEQ ID NO: 4. In some embodiments, the polypeptide is part of a binding construct, and the polypeptide is operatively connected with a second polypeptide that binds at least one growth factor selected from the group consisting of VEGF-A, VEGF-B, VEGF-C, VEGF-D, VEGF-E, PIGF, PDGF-A, PDGF-B, PDGF-C, and PDGF-D. In some embodiments, the second polypeptide is selected from the group consisting of a polypeptide comprising a vascular endothelial growth factor receptor extracellular domain fragment, a platelet derived growth factor receptor extracellular domain fragment, and a polypeptide comprising an antigen binding fragment of an antibody that immunoreacts with the at least one of said growth factors. In some embodiments, at least one of the polypeptides is encoded by a polynucleotide comprising a nucleotide sequence selected from the group consisting of SEQ ID NOS: 21, 23, and 25.

In still another aspect, the invention provides a binding construct comprising a first polypeptide operatively connected to a second polypeptide. The first and second polypeptides each binds at least one growth factor selected from the group consisting of VEGF-A, VEGF-B, VEGF-C, VEGF-D, VEGF-E, PIGF, PDGF-A, PDGF-B, PDGF-C, and PDGF-D polypeptides. The amino acid sequence of the first polypeptide differs from the amino acid sequence of the second polypeptide. The first and second polypeptides comprise members independently selected from the group consisting of:
(a) a polypeptide comprising an amino acid sequence at least 90% identical to the VEGFR-1 extracellular domain amino acid sequence comprising positions 27-758 of SEQ ID NO: 2;
(b) a fragment of (a) that binds VEGF-A, VEGF-B, or PIGF;
(c) a polypeptide comprising an amino acid sequence at least 90% identical to the VEGFR-2 extracellular domain amino acid sequence comprising positions 20-764 of SEQ ID NO: 4;
(d) a fragment of (c) that binds VEGF-A, VEGF-C, VEGF-E or VEGF-D;
(e) a polypeptide comprising an amino acid sequence at least 90% identical to the VEGFR-3 extracellular domain amino acid sequence comprising residues 24-775 of SEQ ID NO: 6;
(f) a fragment of (e) that binds VEGF-C or VEGF-D;
(g) a polypeptide comprising an amino acid sequence at least 90% identical to the neuropilin-1 extracellular domain amino acid sequence comprising residues 22-856 of SEQ ID NO: 113;
(h) a fragment of (g) that binds VEGF-A, VEGF-B, VEGF-C, VEGF-E, or PIGF;
(i) a polypeptide comprising an amino acid sequence at least 90% identical to the neuropilin-2 extracellular domain amino acid sequence comprising residues 21-864 of SEQ ID NO: 115;
(j) a fragment of (i) that binds VEGF-A, VEGF-C, or PIGF;
(k) a polypeptide comprising an amino acid sequence at least 90% identical to the platelet derived growth factor receptor alpha extracellular domain amino acid sequence comprising residues 24-524 of SEQ ID NO: 117;
(l) a fragment of (k) that binds PDGF-A, PDGF-B, or PDGF-C;
(m) a polypeptide comprising an amino acid sequence at least 90% identical to the platelet derived growth factor beta extracellular domain amino acid sequence comprising residues 33 to 531 of SEQ ID NO: 119;
(n) a fragment of (m) that binds PDGF-B or PDGF-D; and
(o) a polypeptide comprising an antigen binding fragment of an antibody that binds to at least one growth factor selected from the group consisting of VEGF-A, VEGF-B, VEGF-C, VEGF-D, VEGF-E, PIGF, PDGF-A, PDGF-B, PDGF-C, and PDGF-D.

Still further examples of polypeptides that comprise binding units of the invention are antibodies and antibody fragments that immunoreact with one or more receptors selected frm VEGFR-1, VEGFR-2, VEGFR-3, PDGFR-alpha, and PDGFR-beta.

In one embodiment, the binding construct of the invention comprises a first polypeptide comprising a fragment of a polypeptide comprising an amino acid sequence at least 90% identical to the VEGFR-2 extracellular domain amino acid sequence comprising positions 20-764 of SEQ ID NO: 4, wherein the fragment binds VEGF-A, VEGF-C, VEGF-E or VEGF-D. Optionally, the binding construct further comprises a second polypeptide comprising a fragment of a polypeptide comprising an amino acid sequence at least 90% identical to the VEGFR-1 extracellular domain amino acid sequence comprising positions 27-758 of SEQ ID NO: 2; wherein the fragment binds VEGF-A, VEGF-B, or PIGF. Additionally, the binding construct optionally further comprises a third polypeptide operatively connected to the first or second polypeptide, wherein the third polypeptide comprises a fragment of a polypeptide comprising an amino acid sequence at least 90% identical to the VEGFR-3 extracellular domain amino acid sequence comprising residues 24-775 of SEQ ID NO: 6, wherein the fragment binds VEGF-C or VEGF-D.

As described herein in greater detail, the extracellular domain of VEGFR or PDGFR have immunoglobulin-like domain structure. In a related embodiment, the binding construct of the invention comprises a first, second and third polypeptide as described above, wherein: (a) the first polypeptide comprises an amino acid sequence at least 90% identical to a fragment of the VEGFR-2 extracellular domain, wherein the fragment comprises immunoglobulin-like domain 2 amino acid sequence; (b) the second polypeptide comprises an amino acid sequence at least 90% identical to a fragment of the VEGFR-1 extracellular domain, wherein the fragment comprises immunoglobulin-like domain 3 amino acid sequence; and (c) the third polypeptide comprises an amino acid sequence at least 90% identical to a fragment of the VEGFR-3 extracellular domain, wherein said fragment comprises VEGFR-3 immunoglobulin-like domain 1 amino acid sequence.

In another aspect, the invention involves use of a binding construct comprising: a) a first amino acid sequence at least 90% identical to a fragment of the VEGFR-3 extracellular domain, wherein said fragment comprises VEGFR-3 immunoglobulin-like domain 1 amino acid sequence; (b) a second amino acid sequence at least 90% identical to a fragment of the VEGFR-2 extracellular domain, wherein the fragment comprises immunoglobulin-like domain 2 amino acid sequence; and, (c) a third amino acid sequence at least 90% identical to a fragment of the VEGFR-1 extracellular domain, wherein the fragment comprises immunoglobulin-like domain 3 amino acid sequence; wherein the first, second, and third amino acid sequences are operatively connected, and wherein the binding construct binds to at least VEGF-A and VEGF-C. In one embodiment, the binding construct comprises an amino acid sequence at least 95% identical to the amino acid sequence set out in SEQ ID NO: 128. In a related embodiment, the binding construct comprises the amino acid sequence of SEQ ID NO: 128.

In some embodiments, the binding construct of the invention comprises a first polypeptide comprising a fragment of a polypeptide comprising an amino acid sequence at least 90% identical to the VEGFR-3 extracellular domain amino acid sequence comprising residues 24-775 of SEQ ID NO: 6, wherein the fragment binds VEGF-C or VEGF-D. It is contemplated that the binding construct of the invention comprises a second polypeptide comprising a fragment of a polypeptide comprising an amino acid sequence at least 90% identical to the VEGFR-2 extracellular domain amino acid sequence comprising positions 20-764 of SEQ ID NO: 4, wherein the fragment binds VEGF-A, VEGF-C, VEGF-E or VEGF-D.

In a related embodiment, the binding construct of the invention comprises a first and second polypeptide as described above, wherein: (a) the first polypeptide comprises an amino acid sequence at least 90% identical to a fragment of the VEGFR-3 extracellular domain, wherein said fragment comprises VEGFR-3 immunoglobulin-like domain 1 amino acid sequence; and, (b) the second polypeptide comprises an amino acid sequence at least 90% identical to a fragment of the VEGFR-2 extracellular domain, wherein the fragment comprises immunoglobulin-like domains 2 and 3 amino acid sequence.

In another aspect, the invention provides a binding construct comprising: a) a first amino acid sequence at least 90% identical to a fragment of the VEGFR-3 extracellular domain, wherein said fragment comprises VEGFR-3 immunoglobulin-like domain 1 amino acid sequence; and, (b) a second amino acid sequence at least 90% identical to a fragment of the VEGFR-2 extracellular domain, wherein the fragment comprises immunoglobulin-like domain 2 amino acid sequence; and an immunoglobulin-like domain 3 amino acid sequence; wherein the first, second, and third amino acid sequences are operatively connected, and wherein the binding construct binds to at least VEGF-A and VEGF-C. It is further contemplated that the construct binds VEGF-D. In one embodiment, the binding construct comprises an amino acid sequence at least 95% identical to the amino acid sequence set out in SEQ ID NO: 125. In a related embodiment, the binding construct comprises the amino acid sequence of SEQ ID NO: 125.

In some variations, the binding unit or units of a binding comprise antibodies or antibody antigen binding fragments. In some embodiments, the binding construct comprises at least one non-antigen binding fragment binding unit. In some embodiments, the binding units all comprise antigen binding fragments of antibodies. Exemplary Bispecific antibodies are provided in U.S. patent application Ser. No. 11/075,400, published as U.S. Patent Publication No. 2005/0282233, and related, co-filed International Patent Application No. PCT/US2005/007742, published as WO 2005/087812 (Attorney Docket No. 28967/39820B), both applications incorporated herein by reference it their entirety. Antibodies that target the growth factors identified herein, and antibodies that target the receptors identified herein, all are useful for practicing the invention. Monoclonal antibody therapeutics are preferred. Humanized and fully human antibodies are highly preferred, as are fragments of such antibodies.

One aspect of the invention is a method for inhibiting allograft rejection or graft-related arteriosclerosis comprising administering to a mammalian subject in need of said inhibition a binding construct according to the invention, in an amount effective to inhibit the allograft rejection or the arteriosclerosis.

The method may also comprise the step of screening an organ transplant recipient mammal to identify elevated levels of at least one growth factor selected from the group consisting of VEGF-A, VEGF-B, VEGF-C, VEGF-D, VEGF-E, PIGF, PDGF-A, PDGF-B, PDGF-C, and PDGF-D polypeptides. In some embodiments, the screening step comprises obtaining a serum sample, a fluid sample, or a tissue sample from the transplanted organ and detecting elevated levels of at least one growth factor selected from the group consisting of VEGF-A, VEGF-B, VEGF-C, VEGF-D, VEGF-E, PIGF, PDGF-A, PDGF-B, PDGF-C, and PDGF-D polypeptides, or elevated levels of at least one receptor capable of binding the same.

The methods of the invention may also be carried out with another therapeutic. For example, other therapeutics that may be used alone, or in combination with the binding constructs of the invention, include anti-sense RNA, RNA interference, bispecific antibodies, other antibody types, and small molecules, e.g., chemotherapeutic agents, which target growth factors and/or their receptors. Combination therapies are preferably synergistic, but they need not be, and additive therapies are also considered aspects of the invention.

In addition to their use in methods, the binding constructs may be combined or packaged with other therapeutics in kits or as unit doses.

This summary of the invention is not intended to be limiting or comprehensive, and additional embodiments are described in the drawings and detailed description, including the examples. All such embodiments are aspects of the invention. Moreover, for the sake of brevity, various details that are applicable to multiple embodiments have not been repeated for every embodiment. Variations reflecting combinations and rearrangements of the embodiments described herein are intended as aspects of the invention. In addition to the foregoing, the invention includes, as an additional aspect, all embodiments of the invention narrower in scope in any way than the variations specifically mentioned above. For example, for aspects described as a genus or range, every subgenus, subrange or species is specifically contemplated as an embodiment of the invention.

### BRIEF DESCRIPTION OF THE DRAWING

FIG. 1 shows that VEGFR-3 inhibition markedly improves long-term survival of rat cardiac allografts in suboptimally-immunosuppressed recipients.

### DETAILED DESCRIPTION

The present invention provides binding constructs, compositions, and materials and methods for making and using the same. The binding constructs bind growth factors that have been shown or are hypothesized to contribute to allograft rejection or arteriosclerosis in allograft recipients in vivo, and are useful for inhibiting those effects.

### I. Binding Constructs

For the purposes of this invention, a “binding construct” comprises one or more binding units associated with each other by covalent or other forms of attachment. A “binding unit” binds a growth factor receptor or a growth factor ligand, i.e., binds to one or more growth factor polypeptides or growth factor receptor polypeptides, and preferably does so with high affinity. A binding unit preferably comprises at least one peptide or polypeptide, but other embodiments are possible as well, including organic small molecules, aptamers, and combinations of the same. While a binding unit preferably comprises a single polypeptide, it may comprise multiple polypeptides if a single polypeptide is not sufficient for binding a particular growth factor. When more than one binding unit or polypeptide segment is in a given binding construct, the binding units may be joined directly (i.e., through a covalent bond, e.g., a peptide, ester, or sulfhydryl bond, or non-covalently, e.g., hydrophobically) together via a linker. A binding construct may further include a heterologous peptide or other chemical moieties. Such additions are can modify binding construct properties such as stability, solubility, toxicity, serum half-life, immunogenicity, detectability, or other properties.

The term “high affinity” is used in a physiological context pertaining to the relative affinity of the binding construct for the growth factor ligand(s) or receptor(s) in vivo in a mammal, such as a laboratory test animal, a domesticated farm or pet animal, or a human. The targeted growth factors of the invention, e.g., the VEGF/PDGF family members, have characteristic affinities for their receptors in vivo, typically measured in terms of sub-nanomolar dissociation constants (K_{d}). For the purposes of this invention, a binding construct can bind to its target growth factor(s) or receptor(s) with a K_{d} less than or equal to 1000 times the K_{d} of the natural growth factor-receptor pair, while retaining the specificity of the natural pair. A binding unit that binds a growth factor with a K_{d} less than or equal to 10 times the K_{d} of the natural growth factor-receptor pair, while retaining the specificity of the natural pair, is considered high affinity. While high affinity is preferred, it is not a requirement. In a preferred embodiment, the affinity of the binding unit for the growth factor or receptor equals or exceeds the affinity of the natural receptor for the growth factor (or vice versa). Such affinities may be readily determined using conventional techniques, such as by using a BIAcore instrument or by radioimmunoassay using radiolabeled target antigen. Affinity data may be analyzed, for example, by the method of Scatchard et al., Ann N.Y. Acad. Sci., 51:660 (1949).

By binding activity is meant the ability to bind to a ligand, receptor, or binding construct, and does not require the retention of biological activity in so far as enzymatic activity or signaling is concerned. Binding may include either binding to a monomer or a dimer, homodimers or heterodimers, whether of receptors or ligands. Polypeptides for use according to the present invention can be used in the form of a protein dimer, particularly a disulfide-linked dimer. Mechanistic descriptions of binding constructs, e.g., as ligand traps, are not meant to be limiting. For example, a binding construct comprising a receptor extracellular domain fragment may function by forming inactive dimers with an endogenous receptor monomer.

In some embodiments, a binding construct comprises a first binding unit (e.g., a polypeptide) operatively associated with a second binding unit (e.g., a polypeptide), wherein each binding unit binds a growth factor selected from the group consisting of VEGF-A, VEGF-B, VEGF-C, VEGF-D, VEGF-E, PIGF, PDGF-A, PDGF-B, PDGF-C, PDGF-D, D1701 VEGF, NZ2 VEGF, NZ7 VEGF, and fallotein. In some embodiments the first and second binding units act together to bind a single ligand molecule (wherein the ligand may comprise a monomer or dimer). In some embodiments, the binding units act independently, i.e., each polypeptide binds a separate ligand molecule. In some embodiments, the first and second binding units are capable of either acting together or acting independently to bind one or more ligand polypeptides. In some embodiments, a binding unit of a first binding construct is able to interact with a binding unit on a second binding construct, e.g., to form dimers between binding units.

In some embodiments, a binding construct comprises a first binding unit operatively associated with a second binding unit, wherein each binding unit binds to a growth factor receptor selected from the group consisting of VEGFR-1, VEGFR-2, VEGFR-3, PDGFR-alpha, PDGFR-beta, and the neuropilins.

In some embodiments, the binding construct comprises a first polypeptide operatively connected to a second polypeptide, wherein the first and second polypeptides each binds at least one growth factor selected from the group consisting of VEGF-A, VEGF-B, VEGF-C, VEGF-D, VEGF-E, and PIGF polypeptides; or bind at least one growth factor receptor selected from VEGFR-1, VEGFR-2, VEGFR-3, PDGFR-alpha, PDGFR-beta, and the neuropilins, wherein the amino acid sequence of the first polypeptide differs from the amino acid sequence of the second polypeptide; and wherein the first and second polypeptides comprise members independently selected from the group consisting of:
(a) a polypeptide comprising an amino acid sequence at least 35% identical to the VEGFR-1 extracellular domain amino acid sequence comprising positions 27-758 of SEQ ID NO: 2;
(b) a fragment of (a) that binds VEGF-A, VEGF-B, or PIGF;
(c) a polypeptide comprising an amino acid sequence at least 35% identical to the VEGFR-2 extracellular domain amino acid sequence comprising positions 20-764 of SEQ ID NO: 4;
(d) a fragment of (c) that binds VEGF-A, VEGF-C, VEGF-E or VEGF-D;
(e) a polypeptide comprising an amino acid sequence at least 35% identical to the VEGFR-3 extracellular domain amino acid sequence comprising residues 24-775 of SEQ ID NO: 6;
(f) a fragment of (e) that binds VEGF-C or VEGF-D;
(g) a polypeptide comprising an amino acid sequence at least 35% identical to the neuropilin-1 extracellular domain amino acid sequence comprising residues 22-856 of SEQ ID NO: 113;
(h) a fragment of (g) that binds VEGF-A, VEGF-B, VEGF-C, VEGF-E, or PIGF;
(i) a polypeptide comprising an amino acid sequence at least 35% identical to the neuropilin-2 extracellular domain amino acid sequence comprising residues 21-864 of SEQ ID NO: 115;
(j) a fragment of (i) that binds VEGF-A, VEGF-C, or PIGF;
(k) a polypeptide comprising an amino acid sequence at least 35% identical to the platelet derived growth factor receptor alpha extracellular domain amino acid sequence comprising residues 24-524 of SEQ ID NO: 117;
(l) a fragment of (k) that binds PDGF-A, PDGF-B, or PDGF-C;
(m) a polypeptide comprising an amino acid sequence at least 35% identical to the platelet derived growth factor beta extracellular domain amino acid sequence comprising residues 33 to 531 of SEQ ID NO: 119;
(n) a fragment of (m) that binds PDGF-B or PDGF-D;
(o) an antibody that binds to at least one growth factor or receptor selected from the group consisting of VEGF-A, VEGF-B, VEGF-C, VEGF-D, VEGF-E, PIGF, PDGF-A, PDGF-B, PDGF-C, PDGF-D; VEGFR-1, VEGFR-2, VEGFR-3, PDGFR-alpha, and PDGFR-beta;
(p) a polypeptide comprising an antigen binding fragment of an antibody that binds to at least one growth factor selected from the group consisting of VEGF-A, VEGF-B, VEGF-C, VEGF-D, VEGF-E, PIGF, PDGF-A, PDGF-B, PDGF-C, and PDGF-D; or of an antibody that binds to at least one growth factor receptor selected from the group consisting of VEGFR-1, VEGFR-2, VEGFR-3, PDGFR-alpha, and PDGFR-beta;
(q) a polypeptide that binds at least one growth factor selected from the group consisting of VEGF-A, VEGF-B, VEGF-C, VEGF-D, VEGF-E, PIGF, PDGF-A, PDGF-B, PDGF-C, and PDGF-D polypeptides, wherein the polypeptide is generated using phage display;
(r) compounds that comprises peptide fragments of one or more of VEGF-A, VEGF-B, VEGF-C, VEGF-D, VEGF-E, PIGF, PDGF-A, PDGF-B, PDGF-C, and PDGF-D, and that inhibit the binding between such growth factors and their receptors; and
(s) an organic molecule that mimics the binding properties of (a)-(r).

In some embodiments, the binding units all comprise antigen binding fragments. Exemplary bispecific antibodies are provided in U.S. patent application Ser. No. 11/075,400, published as U.S. Patent Publication No. 2005/0282233, and related International Patent Application No. PCT/US2005/007742, published as WO 2005/087812 (Attorney Docket No. 28967/39820B), both applications incorporated herein by reference it their entirety.

In some embodiments, one or more of the polypeptides of a binding construct is replaced with another type of molecule, e.g., a nucleic acid, that mimics the binding properties of any of the polypeptides described above in (a) through (p). Such nucleic acids include, for example, aptamers.

### A. Binding Units

The growth factors that are the targets of the binding constructs of the invention exert their physiological effects in vivo by binding to the extracellular domains of growth factor receptors. Accordingly, growth factor receptors and fragments thereof constitute examples of binding units. Exemplary human nucleotide and amino acid sequences, for relevant ligands and receptors are set forth in the sequence listing as summarized below:

TABLE 1A
RECEPTOR SEQUENCES

| RECEPTOR | SEQ ID NOS: |
|---|---|
| VEGFR-1 | 1 and 2 |
| VEGFR-2 | 3 and 4 |
| VEGFR-3 short | 5 and 6 |
| VEGFR-3 long | 120 and 121 |
| PDGFR-α | 116 and 117 |
| PDGFR-β | 118 and 119 |
| Neuropilin-1 | 112 and 113 |
| Neuropilin-2 | 114 and 115 |

TABLE 1B
LIGAND SEQUENCES

| LIGAND | SEQ ID NOS: |
|---|---|
| VEGF-A | 80 and 81 |
| VEGF-A 232 isoform | 90 and 91 |
| VEGF-B isoform 1 | 94 and 95 |
| VEGF-B isoform 2 | 96 and 97 |
| VEGF-C | 82 and 83 |
| VEGF-D | 86 and 87 |
| VEGF-E (NZ7) | 88 and 89 |
| PlGF | 84 and 85 |
| D1701 VEGF | 92 and 93 |
| PDGF-A | 98 and 99 |
| PDGF-B | 100 and 101 |
| PDGF-C | 102 and 103 |
| PDGF-D | 104 and 105 |

Other VEGF growth factors members include snake venom VEGFs (e.g., EMBL. AY033151, AY033152, and AY42981), various VEGF-E (orf virus VEGF homologs, some of which are presented in Table 1B) molecules including VEGF-E NZ2 [S67520], VEGF-E NZ7, VEGF-E D1701, VEGF-E Orf-11, and VEGF-E OV-IA82. [See generally, WO 00/25085.]

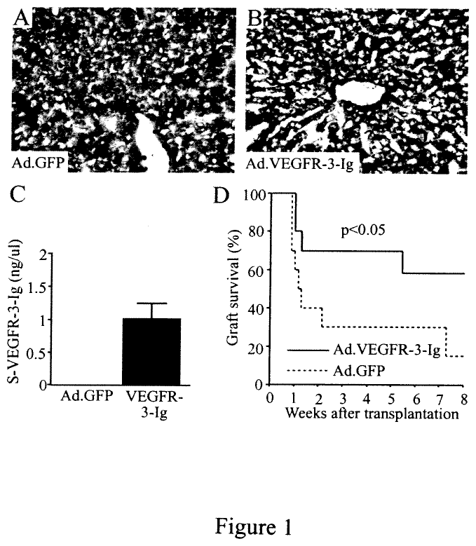

Members of the PDGF/VEGF family are characterized by a number of structural motifs including a conserved PDGF motif defined by the sequence: P-[PS]-C—V-X(3)-R-C-[GSTA]-G-C-C(SEQ ID NO: 111), where the brackets indicate a variable position that can be any one of the amino acids within the brackets. The number contained within the parentheses indicates the number of amino acids that separate the “V” and “R” residues. This conserved motif falls within a large domain of 70-150 amino acids defined in part by eight highly conserved cysteine residues that form inter- and intramolecular disulfide bonds. This domain forms a cysteine knot motif composed of two disulfide bonds which form a covalently linked ring structure between two adjacent 13 strands, and a third disulfide bond that penetrates the ring [see for example, FIG. 1 in Muller et al., Structure 5:1325-1338 (1997)], similar to that found in other cysteine knot growth factors, e.g., transforming growth factor-β (TGF-β). The amino acid sequence of all known PDGF/VEGF proteins, with the exception of VEGF-E, contains the PDGF domain. The PDGF/VEGF family proteins are predominantly secreted glycoproteins that form either disulfide-linked or non-covalently bound homo- or heterodimers whose subunits are arranged in an anti-parallel manner [Stacker and Achen, Growth Factors 17:1-11 (1999); Muller et al., Structure 5:1325-1338 (1997)]. Binding constructs of the invention include those that bind VEGF/PDGF growth factor monomers, homodimers, and heterodimers.

The VEGF subfamily is composed of members that share a VEGF homology domain (VHD) characterized by the sequence: C-X(22-24)-P-[PSR]-C—V-X(3)-R-C-[GSTA]-G-C-C-X(6)-C-X(32-41)-C. (SEQ ID: 110) The VHD domain, determined through analysis of the VEGF subfamily members, comprises the PDGF motif but is more specific. The VEGF subfamily of growth factors and receptors regulate the development and growth of the vascular endothelial system. VEGF family members include, but are not limited to VEGF-A, VEGF-B, VEGF-C, VEGF-D and PIGF [Li, X. and U. Eriksson, “Novel VEGF Family Members: VEGF-B, VEGF-C and VEGF-D,” Int. J. Biochem. Cell. Biol., 33(4):421-6 (2001))] Other VEGFs are bacterial or viral, the “VEGF-Es.” Other VEGFs are derived from snake venom, the “NZ” series. [See e.g., Komori, et al. Biochemistry, 38(36):11796-803 (1999); Gasmi, et al., Biochem Biophys Res Commun, 268(1):69-72 (2002); Gasmi, et al., J Biol Chem; 277(33):29992-8 (2002); de Azevedo, et al., J. Biol. Chem., 276: 39836-39842 (2001)].

At least seven cell surface receptors that interact with PDGF/VEGF family members have been identified. These include PDGFR-α [See e.g., GenBank Acc. No. NM006206; Swiss Prot No. P16234], PDGFR-β [See e.g., GenBank Acc. No. NM002609; Swiss Prot. No. P09619], VEGFR-1/Flt-1 (fins-like tyrosine kinase-1; hereinafter “R-1”) [GenBank Acc. No. X51602; De Vries, et al., Science 255:989-991 (1992)]; VEGFR-2/KDR/Flk-1 (kinase insert domain containing receptor/fetal liver kinase-1, hereinafter “R-2”) [GenBank Acc. Nos. X59397 (Flk-1) and L04947 (KDR); Terman, et al., Biochem. Biophys. Res. Comm. 187:1579-1586 (1992); Matthews, et al., Proc. Natl. Acad. Sci. USA 88:9026-9030 (1991)]; VEGFR-3/Flt4 (fins-like tyrosine kinase 4; hereinafter “R-3”) [U.S. Pat. No. 5,776,755 and GenBank Acc. No. X68203 and S66407; Pajusola et al., Oncogene 9:3545-3555 (1994); Hughes, et al., J. Mol. Evol. 52(2):77-79 (2001); Pajusola, et al., Oncogene 8(11):2931-37) (1993); Borg, et al., Oncogene 10(5):973-984 (1995), neuropilin-1 [Gen Bank Acc. No. NM003873], and neuropilin-2 [Gen Bank Acc. No. NM003872; SwissProt 060462]. The two PDGF receptors mediate signaling of PDGFs. Non-human VEGF and PDGF receptors may also be employed as part of the invention, e.g., chicken VEGFR-1 may be used alone or in hybrid form with human R-1 for improved expression.

VEGF121, VEGF165, VEGF-B, PIGF-1 and PIGF-2 bind VEGF-R1; VEGF121, VEGF145, VEGF165, (fully processed mature) VEGF-C, (fully processed mature) VEGF-D, VEGF-E, and NZ2 VEGF bind VEGF-R2; VEGF-C and VEGF-D bind VEGFR-3; VEGF165, VEGF-C, PIGF-2, and NZ2 VEGF bind neuropilin-1; and VEGF165 and VEGF-C binds neuropilin-2. [Neufeld, et al., FASEB. J. 13:9-22 (1999); Stacker and Achen, Growth Factors 17:1-11 (1999); Ortega, et al., Fron. Biosci. 4:141-152 (1999); Zachary, Intl. J. Biochem. Cell. Bio. 30:1169-1174 (1998); Petrova, et al., Exp. Cell. Res. 253:117-130 (1999); U.S. Pat. Appl. Pub. No. 20030113324]. PDGF-A, PDGF-B, and PDGF-C bind PDGFR-α. PDGF-B and PDGF-D bind PDGF-β.

Both the ligands and the receptors generally exist as dimers, including both homodimers and heterodimers. Such dimers can influence binding. For example, for the PDGFs, PDGF-AA binds PDGFR-α/α. PDGF-AB and PDGF-CC bind PDGFR-α/α and PDGFR-α/β. PDGFR-BB binds both of the homodimers and the heterodimeric PDGF receptor. PDGF-DD binds PDGF receptor heterodimers and beta receptor homodimers. [See, e.g., Pietras, et al., Cancer Cell, 3:439-443 (2003).] VEGF-A can heterodimerize with VEGF-B and PIGF. The VEGFs, PDGFs, and PIGFs, may exist as two or more isoforms, e.g., splice variants, and not all isoforms of a particular growth factor will share the same binding profile, or ability to dimerize with particular molecules. Certain isoforms of the same growth factor may also dimerize with each other. For example the 167 and 186 isoforms of VEGF-B can heterodimerize with each other.

Growth factor receptor tyrosine kinases generally comprise three principal domains: an extracellular domain, a transmembrane domain, and an intracellular domain. The extracellular domain binds ligands, the transmembrane domain anchors the receptor to a cell membrane, and the intracellular domain possesses one or more tyrosine kinase enzymatic domains and interacts with downstream signal transduction molecules. The vascular endothelial growth factor receptors (VEGFRs) and platelet derived growth factor receptors (PDGFRs) bind their ligand through their extracellular domains (ECDs), which are comprised of multiple immunoglobulin-like domains (Ig-domains). Ig-domains are identified herein using the designation “D #.” For example “D1” refers to the first Ig-domain of a particular receptor ECD. “D1-3” refers to a construct containing at least the first three Ig-domains, and intervening sequence between domains 1 and 2 and 2 and 3, of a particular construct. Table 2 defines the boundaries of the Ig-domains for VEGFR-1, VEGFR-2, and VEGFR-3 of the invention. These boundaries are significant as the boundaries chosen can be used to form constructs, and so can influence the binding properties of the resulting constructs. This relationship is discussed in Example 1.

The complete ECD of PDGFRs and VEGFRs is not required for ligand (growth factor) binding. The ECD of VEGFR-1 (R-1) and VEGFR-2 (R-2) consists of seven Ig-like domains and the ECD of VEGFR-3 (R-3) has six intact Ig-like domains—D5 of R-3 is cleaved post-translationally into disulfide linked subunits leaving VEGFR-3. Veikkola, T., et al., Cancer Res. 60:203-212 (2000). In general, receptor fragments of at least the first three Ig-domains for this family are sufficient to bind ligand. The PDGFRs have five Ig-domains.

TABLE 2
IMMUNOGLOBULIN-LIKE DOMAINS FOR
VEGFR-1, VEGFR-2 AND VEGFR-3

| | R-1 | R-1 | R-2 | R-2 | R-3 | R-3 |
|---|---|---|---|---|---|---|
| | SEQ ID | SEQ ID | SEQ ID | SEQ ID | SEQ ID | SEQ ID |
| | NO: 1 | NO: 2 | NO: 3 | NO: 4 | NO: 5 | NO: 6 |
| | positions | positions | positions | positions | positions | positions |
| D1 | 394-580 | 49-111 | 145-316 | 48-105 | 158-364 | 47-115 |
| D2 | 709-880 | 154-211 | 436-610 | 145-203 | 479-649 | 154-210 |
| D3 | 990-1192 | 248-315 | 724-931 | 241-310 | 761-961 | 248-314 |
| D4 | 1303-1474 | 352-409 | 1039-1204 | 346-401 | 1070-1228 | 351-403 |
| D5 | 1957-1864 | 450-539 | 1321-1600 | 440-533 | 1340-1633 | 441-538 |
| D6 | 1966-2167 | 573-640 | 1699-1936 | 566-645 | 1739-1990 | 574-657 |
| D7 | 2281-2452 | 678-735 | 2050-2221 | 683-740 | 2102-2275 | 695-752 |

In some embodiments, a binding unit of a binding construct comprises the ECD of a growth factor receptor. A binding unit may comprise at least one Ig-domain of a VEGFR as described in Table 2, to as many as seven. Ig-domain information for PDGFR-α and PDGFR-β is provided in Lokker, et al., J. Biol. Chem. 272: 33037-33044 (1997), which is incorporated by reference in its entirety. A binding unit may include sequence before the N-terminal most Ig-domain, may include sequence beyond the C-terminal most Ig-domain, and may include sequence between the Ig-domains as well. Binding units may also comprise variants, e.g., with one or more amino acid substitutions, additions, or deletions of an amino acid residue. Binding units also may comprise chimeras, e.g., combinations of Ig-domains from different receptors. In some embodiments, the first or second polypeptide comprises a receptor fragment comprising at least the first three Ig domains of a receptor tyrosine kinase.

The binding of a binding unit to a particular growth factor ligand refers to the ability to bind at least one natural isoform of at least one target growth factor, especially processed forms that are secreted from cells and circulate in vivo and/or bind heparin moieties. For example, “capable of binding VEGF-A” refers to the ability to bind at least one isoform of VEGF-A under physiological conditions. At least five human VEGF-A isoforms of 121, 145, 165, 189 or 206 amino acids in length (VEGF121-VEGF206), encoded by distinct mRNA splice variants, have been described, all of which are capable of stimulating mitogenesis in endothelial cells. [See generally, Ferrara, J. Mol. Med. 77:527-543 (1999).] Two VEGF-B isoforms generated by alternative mRNA splicing exist, VEGF-B186 and VEGF-B167, with the first isoform accounting for about 80% of the total VEGF-B transcripts [Li, X., et al., Growth Factor, 19:49-59 (2001); Grimmond, et al., Genome Res., 6:124-131 (1996); Olofsson, et al., J. Biol. Chem., 271:19310-19317 (1996).] Three isoforms of PIGF produced by alternative mRNA splicing have been described [Hauser, et al., Growth Factors 9:259-268 (1993); Maglione, et al., Oncogene 8:925-931 (1993)]. PDGF-A and PDGF-B can homodimerize or heterodimerize to produce three different isoforms: PDGF-AA, PDGF-AB, or PDGF-BB.

The term “identity”, as known in the art, refers to a relationship between the sequences of two or more polypeptide molecules or two or more nucleic acid molecules, as determined by comparing the sequences. In the art, “identity” also means the degree of sequence relatedness nucleic acid molecules or polypeptides sequences, as the case may be, as determined by the match between strings of two or more nucleotide or two or more amino acid sequences. “Identity” measures the percent of identical matches between the smaller of two or more sequences with gap alignments (if any) addressed by particular a mathematical model of computer program (i.e., “algorithms”). Appropriate algorithms for determining the percent identies of the invention include BLASTP and BLASTN, using the most common and accepted default parameters.

### 1. VEGFR-1-Derived Binding Units

In some embodiments, a binding unit comprises a polypeptide similar or identical in amino acid sequence to a VEGFR-1 polypeptide or fragment thereof, preferably from the same species as the targeted growth factor(s). Thus, for binding to human growth factors, a binding unit preferably comprises a polypeptide that comprises an amino acid similar or identical to a fragment of SEQ ID NO: 2, wherein the fragment and the polypeptide binds one or more growth factors selected from the group consisting of VEGF-A, VEGF-B, and PIGF. The fragment minimally comprises enough of the VEGFR-1 sequence to bind the ligand, and may comprise the complete receptor. Extracellular domain fragments are preferred. Preferred polypeptides have an amino acid sequence at least 80% identical to a ligand binding fragment thereof. Fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated.

Preferred polypeptides may also be described as having an amino acid sequence encoded by a nucleic acid sequence at least 80% identical to a fragment of SEQ ID NO:1 encoding a ligand binding fragment of VEGFR-1. Nucleic acid fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated. A genus of similar polypeptides can alternatively be defined by the ability of encoding polynucleotides to hybridize to the complement of a nucleotide sequence that corresponds to the cDNA sequence encoding the R-1 receptor. For example, a preferred binding unit polypeptide comprises an amino acid sequence that binds one or more R-1 ligands and that is encoded by a nucleotide sequence that hybridizes to the complement of SEQ ID NO: 1 under moderately or highly stringent conditions discussed herein.

Exemplary R1 fragments for use as binding unit polypeptides (or for use as a starting point for designing R-1 analogs) have an amino terminal residue selected from the group consisting of positions 1 to 129 of SEQ ID NO: 2, and a carboxy terminal residue selected from the group consisting of positions 229 to 758 of SEQ ID NO: 2, wherein the VEGFR-1 fragment binds at least one of VEGF-A, VEGF-B, and PIGF.

### 2. VEGFR-2-Derived Binding Units

In some embodiments, a binding unit comprises a polypeptide similar or identical in amino acid sequence to a VEGFR-2 polypeptide or fragment thereof, preferably from the same species as the targeted growth factor(s). Thus, for binding to human growth factors, a binding unit preferably comprises a polypeptide that comprises an amino acid similar or identical to a fragment of SEQ ID NO: 4, wherein the fragment and the polypeptide binds one or more growth factors selected from the group consisting of VEGF-A, VEGF-C, VEGF-D, or VEGF-E. The fragment minimally comprises enough of the VEGFR-2 sequence to bind the ligand, and may comprise the complete receptor. Extracellular domain fragments are preferred. Preferred polypeptides have an amino acid sequence at least 80% identical to a ligand binding fragment thereof. Fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated.

Preferred polypeptides may also be described as having an amino acid sequence encoded by a nucleic acid sequence at least 80% identical to a fragment of SEQ ID NO:3 encoding a ligand binding fragment of VEGFR-2. Nucleic acid fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated. A genus of similar polypeptides can alternatively be defined by the ability of encoding polynucleotides to hybridize to the complement of a nucleotide sequence that corresponds to the cDNA sequence encoding the R-2 receptor. For example, a preferred binding unit polypeptide comprises an amino acid sequence that binds one or more R-2 ligands and that is encoded by a nucleotide sequence that hybridizes to the complement of SEQ ID NO: 3 under moderately or highly stringent conditions discussed herein.

Exemplary R2 fragments for use as binding unit polypeptides (or for use as a starting point for designing R-2 analogs) have an amino terminal residue selected from the group consisting of positions 1 to 118 of SEQ ID NO: 4, and a carboxy terminal residue selected from the group consisting of positions 326 to 764 of SEQ ID NO: 4, wherein VEGFR-2 fragment binds at least one of VEGF-A, VEGF-C, VEGF-D, and VEGF-E. Exemplary R2 fragments for use as binding unit polypeptides (or for use as a starting point for designing R-2 analogs) may alternatively have an amino terminal residue selected from the group consisting of positions 1 to 192 of SEQ ID NO: 4, and a carboxy terminal residue selected from the group consisting of positions 393 to 764 of SEQ ID NO: 4, wherein the VEGFR-2 fragment binds at least one of VEGF-A, VEGF-C, VEGF-D, and VEGF-E. Exemplary R2 fragments for use as binding unit polypeptides (or for use as a starting point for designing R-2 analogs) may also have an amino terminal residue selected from the group consisting of positions 1 to 48 of SEQ ID NO: 4, and a carboxy terminal residue selected from the group consisting of positions 214 to 764 of SEQ ID NO: 4, wherein the VEGFR-2 fragment binds at least one of VEGF-A, VEGF-C, VEGF-D, and VEGF-E.

In some embodiments, a binding unit of the binding construct comprises a fragment of R-2, SEQ ID NO: 4, selected from the group consisting of positions 24-326 (SEQ ID NO: 8), 118-326 (SEQ ID NO: 20), positions 118-220 (SEQ ID NO: 22), positions 118-226 (SEQ ID NO: 24), and positions 118-232 (SEQ ID NO: 26). In some embodiments, a binding unit of the binding construct comprises a fragment of R-2, SEQ ID NO: 4, selected from the group consisting of positions 106-240, positions 112-234, positions 114-220, positions 115-220, positions 116-222, positions 117-220, positions 118-221, positions 118-222, positions 118-223, positions 118-224, and positions 118-228. In some embodiments, a binding unit of the binding construct comprises a fragment of R-2, SEQ ID NO: 4, selected from the group consisting of positions 48-203, and 145-310 and 48-310. Exemplary embodiments are also discussed in Example 1.

### 3. VEGFR-3-Derived Binding Units

In some embodiments, a binding unit comprises a polypeptide similar or identical in amino acid sequence to a VEGFR-3 polypeptide or fragment thereof, preferably from the same species as the targeted growth factor(s). Thus, for binding to human growth factors, a binding unit preferably comprises a polypeptide that comprises an amino acid similar or identical to a fragment of SEQ ID NO: 6, where the fragment and the polypeptide binds one or more growth factors selected from the group consisting of VEGF-C and VEGF-D. The fragment minimally comprises enough of the VEGFR-3 sequence to bind the ligand, and may comprise the complete receptor. Extracellular domain fragments are preferred. Preferred polypeptides have an amino acid sequence at least 80% identical to a ligand binding fragment thereof. Fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated. A genus of similar polypeptides can alternatively be defined by the ability of encoding polynucleotides to hybridize to the complement of a nucleotide sequence that corresponds to the cDNA sequence encoding the R-3 receptor.

Preferred polypeptides may also be described as having an amino acid sequence encoded by a nucleic acid sequence at least 80% identical to a fragment of SEQ ID NO:5 encoding a ligand binding fragment of VEGFR-3. Nucleic acid fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated. For example, a preferred binding unit polypeptide comprises an amino acid sequence that binds one or more R-3 ligands and that is encoded by a nucleotide sequence that hybridizes to the complement of SEQ ID NO: 5 under moderately or highly stringent conditions discussed herein.

Exemplary R-3 fragments for use as binding unit polypeptides (or for use as a starting point for designing R-3 analogs) have an amino terminal residue selected from the group consisting of positions 1 to 47 of SEQ ID NO: 6, and a carboxy terminal residue selected from the group consisting of positions 226 to 775 of SEQ ID NO: 6, wherein VEGFR-3 fragment binds at least one of VEGF-C and VEGF-D.

In some embodiments, a binding unit of the binding construct comprises a fragment of R-3, SEQ ID NO: 6, selected from the group consisting of positions 1-226 (SEQ ID NO: 38), positions 1-229 (SEQ ID NO: 36), and positions 1-329 (SEQ ID NO: 44). In some embodiments, a binding unit of the binding construct comprises a fragment of R-3, SEQ ID NO: 6, selected from the group consisting of positions 47-224, positions 47-225, positions 47-226, positions 47-227, positions 47-228, positions 47-229, positions 47-230, positions 47-231, positions 47-232, positions 47-236, positions 47-240, and positions 47-245. In some embodiments, a binding unit of the binding construct comprises a fragment of R-3, SEQ ID NO: 6, selected from the group consisting of positions 47-314, positions 47-210, and positions 47-247. Exemplary embodiments are also discussed in Example 1.

### 4. Neuropilin-1-Derived Binding Units

In some embodiments, a binding unit comprises a polypeptide similar or identical in amino acid sequence to a neuropilin-1 polypeptide or fragment thereof, preferably from the same species as the targeted growth factor(s). Thus, for binding to human growth factors, a binding unit preferably comprises a polypeptide that comprises an amino acid similar or identical to a fragment of SEQ ID NO: 113, where the fragment and the polypeptide binds one or more growth factors selected from the group consisting of VEGF-A, VEGF-B, VEGF-C, VEGF-E, and PIGF. The fragment minimally comprises enough of the neuropilin-1 sequence to bind the ligand, and may comprise the complete receptor. Extracellular domain fragments are preferred. Preferred polypeptides have an amino acid sequence at least 80% identical to a ligand binding fragment thereof. Fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated.

Preferred polypeptides may also be described as having an amino acid sequence encoded by a nucleic acid sequence at least 80% identical to a fragment of SEQ ID NO:112 encoding a ligand binding fragment of neuropilin-1. Nucleic acid fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated. A genus of similar polypeptides can alternatively be defined by the ability of encoding polynucleotides to hybridize to the complement of a nucleotide sequence that corresponds to the cDNA sequence encoding the neuropilin-1 receptor. For example, a preferred binding unit polypeptide comprises an amino acid sequence that binds one or more neuropilin-1 ligands and that is encoded by a nucleotide sequence that hybridizes to the complement of SEQ ID NO: 112 under moderately or highly stringent conditions discussed herein.

Exemplary neuropilin-1 fragments for use as binding unit polypeptides (or for use as a starting point for designing neuropilin-1 analogs) comprise a neuropilin-1 extracellular domain amino acid sequence comprising residues 22-856 of SEQ ID NO: 113, or a portion thereof; wherein the neuropilin-1 fragment and the binding unit bind at least one growth factor selected from the group consisting of VEGF-A, VEGF-B, VEGF-C, VEGF-E, and PIGF.

### 5. Neuropilin-2-Derived Binding Units

In some embodiments, a binding unit comprises a polypeptide similar or identical in amino acid sequence to a neuropilin-2 polypeptide or fragment thereof, preferably from the same species as the targeted growth factor(s). Thus, for binding to human growth factors, a binding unit preferably comprises a polypeptide that comprises an amino acid similar or identical to a fragment of SEQ ID NO: 115, wherein the fragment and the polypeptide binds one or more growth factors selected from the group consisting of VEGF-A, VEGF-C, and PIGF. The fragment minimally comprises enough of the neuropilin-2 sequence to bind the ligand, and may comprise the complete receptor. Extracellular domain fragments are preferred. Preferred polypeptides have an amino acid sequence at least 80% identical to a ligand binding fragment thereof. Fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated.

Preferred polypeptides may also be described as having an amino acid sequence encoded by a nucleic acid sequence at least 80% identical to a fragment of SEQ ID NO: 114 encoding a ligand binding fragment of neuropilin-2. Nucleic acid fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated. A genus of similar polypeptides can alternatively be defined by the ability of encoding polynucleotides to hybridize to the complement of a nucleotide sequence that corresponds to the cDNA sequence encoding the neuropilin-2 receptor. For example, a preferred binding unit polypeptide comprises an amino acid sequence that binds one or more neuropilin-2 ligands and that is encoded by a nucleotide sequence that hybridizes to the complement of SEQ ID NO: 114 under moderately or highly stringent conditions discussed herein.

Exemplary neuropilin-2 fragments for use as binding unit polypeptides comprising residues 21-864 of SEQ ID NO: 115, or a portion thereof; wherein the neuropilin-2 fragment and the binding unit bind at least one growth factor selected from the group consisting of VEGF-A, VEGF-C, and PIGF.

Further neuropilin-1 and -2 species, isoforms, soluble fragments, etc., are provided in WO03/029814, U.S. application Ser. Nos. 10/262,538, 10/669,176, and 60/505,607, which are incorporated by reference in their entireties.

### 6. PDGFR-Alpha-Derived Binding Units

In some embodiments, a binding unit comprises a polypeptide similar or identical in amino acid sequence to a PDGFR-α polypeptide or fragment thereof, preferably from the same species as the targeted growth factor(s). Thus, for binding to human growth factors, a binding unit preferably comprises a polypeptide that comprises an amino acid similar or identical to a fragment of SEQ ID NO: 117, where the fragment and the polypeptide binds one or more growth factors selected from the group consisting of PDGF-A, PDGF-B, and PDGF-C. The fragment minimally comprises enough of the PDGFR-α sequence to bind the ligand, and may comprise the complete receptor. Extracellular domain fragments are preferred. Preferred polypeptides have an amino acid sequence at least 80% identical to a ligand binding fragment thereof. Fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated. A genus of similar polypeptides can alternatively be defined by the ability of encoding polynucleotides to hybridize to the complement of a nucleotide sequence that corresponds to the cDNA sequence encoding the R-α receptor.

Preferred polypeptides may also be described as having an amino acid sequence encoded by a nucleic acid sequence at least 80% identical to a fragment of SEQ ID NO:116 encoding a ligand binding fragment of R-α. Nucleic acid fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated. For example, a preferred binding unit polypeptide comprises an amino acid sequence that binds one or more R-α ligands and that is encoded by a nucleotide sequence that hybridizes to the complement of SEQ ID NO: 116 under moderately or highly stringent conditions discussed herein.

Exemplary R-α fragments for use as binding unit polypeptides (or for use as a starting point for designing R-α analogs) have an amino terminal residue selected from the group consisting of positions 1 to 123 of SEQ ID NO: 117, and a carboxy terminal residue selected from the group consisting of positions 313 to 524 of SEQ ID NO: 117, wherein the PDGFR-α fragment binds at least one of PDGF-A, PDGF-B, and PDGF-C.

### 7. PDGFR-Beta-Derived Binding Units

In some embodiments, a binding unit comprises a polypeptide similar or identical in amino acid sequence to a R-β polypeptide or fragment thereof, preferably from the same species as the targeted growth factor(s). Thus, for binding to human growth factors, a binding unit preferably comprises a polypeptide that comprises an amino acid similar or identical to a fragment of SEQ ID NO: 119, where the fragment and the polypeptide binds one or more growth factors selected from the group consisting of PDGF-B and PDGF-D. The fragment minimally comprises enough of the PDGFR-β sequence to bind the ligand, and may comprise the complete receptor. Extracellular domain fragments are preferred. Preferred polypeptides have an amino acid sequence at least 80% identical to a ligand binding fragment thereof. Fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated. A genus of similar polypeptides can alternatively be defined by the ability of encoding polynucleotides to hybridize to the complement of a nucleotide sequence that corresponds to the cDNA sequence encoding the R-β receptor.

Preferred polypeptides may also be described as having an amino acid sequence encoded by a nucleic acid sequence at least 80% identical to a fragment of SEQ ID NO: 118 encoding a ligand binding fragment of PDGFR-β. Nucleic acid fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated. For example, a preferred binding unit polypeptide comprises an amino acid sequence that binds one or more R-β ligands and that is encoded by a nucleotide sequence that hybridizes to the complement of SEQ ID NO: 118 under moderately or highly stringent conditions discussed herein.

Exemplary R-β fragments for use as binding unit polypeptides (or for use as a starting point for designing R-β analogs) have an amino terminal residue selected from the group consisting of positions 1 to 124 of SEQ ID NO: 119, and a carboxy terminal residue selected from the group consisting of positions 314 to 531 of SEQ ID NO: 119, wherein PDGFR-β fragment binds at least one of PDGF-B and PDGF-D.

### 8. Other Binding Units

Although a binding unit may comprise a polypeptide similar or identical to an extracellular domain fragment of a growth factor receptor tyrosine kinase, other binding units are contemplated as well. In some embodiments, the binding unit is generated using phage display. In some embodiments, the binding unit comprises an antibody. In some embodiments, a binding unit comprises a polypeptide comprising an antibody (antigen binding) fragment, e.g., a domain antibody. Binding units, as well as binding constructs, need not comprise a polypeptide. In some embodiments, the binding construct comprises nucleic acid, e.g., DNA or RNA, such as an aptamer. In some embodiments, the binding construct comprises polysaccharides.

Growth factor binding molecules that have been described in the literature may be used as binding units to construct binding constructs of the inventory including molecules taught by the following: Veikkola, T., et al., Cancer Res. 60:203-212 (2000); Davis-Smyth, T., et al., EMBO J., 15(18): 4919-27 (1996), U.S. Pat. Nos. 5,952,199; 6,100,071; 6,383,486; U.S. Pat. Appl. Nos. 20030092604; Niwa, et al., U.S. Pat. No. 6,348,333; Fairbrother, et al., Biochemistry, 37:17754-64 (1998); Starovasnik, M. et al., J. Mol. Biol., 293: 531-44 (1999); Wiesmann, C., et al., Cell, 91:695-704 (1997); Fuh, et al., J. Biol. Chem., 273(18): 11197-11204 (1998); Shinkai, A. et al., J. Biol. Chem., 273(47):31283-88 (1998); Lu, et al., J. Biol. Chem., 275(19): 14321-14330 (2000); Lu et al., J. Immunological Methods, 230:159-71 (1999); Lu, et al., J. Biol. Chem., 278(44): 43496-43507 (2003); Makkinen, T., et al., Nature Medicine, 7(2), 199-205 (2001); Alitalo, et al., WO 02/060950; Karpanen, T., et al., Cancer Research 61:1786-90 (2001); Liu, et al., U.S. Pat. Appl. Publ. No. 2003/0064053; Kubo, H., et al., Blood, 96(2): 546-553 (2000); Rosen, Hematol. Oncol. Clin. N. Am., 16:1173-1187 (2002); Kaplan, et al., Growth Factors, 14:243-256 (1997); Thomas, et al., U.S. Pat. No. 6,375,929; Kendall and Thomas, PNAS, U.S.A., 90:10705-10709 (1993); Kovesdi, U.S. Pat. Appl. Publ. No. 2003/0053989; Daly, et al., U.S. Pat. Appl. Publ. No.: 2004/0014667; and Lokker, et al., J. Biol. Chem. 272: 33037-33044 (1997). These and other documents cited in this application are incorporated in their entireties. Molecules that have not previously been tested for their ability to bind to a particular growth factor may tested according to the assays provided herein. For example, some of the above documents teach a R-2 fragment that binds VEGF-A. That same molecule may be tested for its ability to bind VEGF-C.

Except as otherwise noted, descriptions supplied for receptors, also apply to receptor fragments and such fragments incorporated into binding constructs as described herein.

The growth factor receptors, from which binding units may be derived, include splice variants and naturally-occurring allelic variations. Allelic variants are well known in the art, and represent alternative forms or a nucleic acid sequence that comprise substitution, deletion or addition of one or more nucleotides, but which do not result in any substantial functional alteration of the encoded polypeptide. Standard methods can readily be used to generate such polypeptides including site-directed mutagenesis of polynucleotides, or specific enzymatic cleavage and ligation. Similarly, use of peptidomimetic compounds or compounds in which one or more amino acid residues are replaced by a non-naturally-occurring amino acid or an amino acid analog that retain binding activity is contemplated. Preferably, where amino acid substitution is used, the substitution is conservative, i.e. an amino acid is replaced by one of similar size and with similar charge properties. As used herein, the term “conservative substitution” denotes the replacement of an amino acid residue by another, biologically similar residue. Examples of conservative substitutions include the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic acid for aspartic acid, or glutamine for asparagine, and the like. Neutral hydrophilic amino acids that can be substituted for one another include asparagine, glutamine, serine and threonine. The term “conservative substitution” also includes the use of a substituted amino acid in place of an unsubstituted amino acid.

Alternatively, conservative amino acids can be grouped as described in Lehninger, (Biochemistry, Second Edition; Worth Publishers, Inc. NY:NY, pp. 71-77 (1975)) as set out in the following:
Non-polar (hydrophobic)
   A. Aliphatic: A, L, I, V, P,
   B. Aromatic: F, W,
   C. Sulfur-containing: M,
   D. Borderline: G.
Uncharged-polar
   A. Hydroxyl: S, T, Y,
   B. Amides: N, Q,
   C. Sulfhydryl: C,
   D. Borderline: G.

Positively Charged (Basic): K, R, H.

Negatively Charged (Acidic): D, E.

### B. Linkers

While binding units may be directly attached to one another (via a peptide, disulfide or other type of covalent bond), the binding constructs of the present invention may further comprise a (one or more) linker that connects together two or more different binding units, e.g., a receptor fragments with another receptor fragment, or even a copy of itself. A linker may also link a binding unit to other substituents described herein. The linker is generally a heterologous protein polypeptide. In some embodiments, the linker comprises a peptide that links the binding units to form a single continuous peptide that can be expressed as a single molecule. Linkers may be chosen such that they are less likely to induce an allergic reaction. Polysaccharides or other moieties also may be used to link binding units to form a binding construct.

More than one linker may be used per binding construct. The linker may be selected for optimal conformational (steric) freedom between the various ligand binding units to allow them to interact with each other if desired, e.g., to form dimers, or to allow them to interact with ligand. The linker may be linear such that consecutive binding units are linked in series, or the linker may serve as a scaffold to which various binding units are attached, e.g., a branched linker. A linker may also have multiple branches, e.g., as disclosed in Tam, J. Immunol. Methods 196:17 (1996). Binding units may be attached to each other or to the linker scaffold via N-terminal amino groups, C-terminal carboxyl groups, side chains, chemically modified groups, side chains, or other means.

Linker peptides may be designed to have sequences that permit desired characteristics. For example, the use of glycyl residues allow for a relatively large degree of conformational freedom, whereas a proline would tend to have the opposite effect. Peptide linkers may be chosen so that they achieve particular secondary and tertiary structures, e.g., alpha helices, beta sheets or beta barrels. Quaternary structure can also be utilized to create linkers that join two binding units together non-covalently. For example, fusing a protein domain with a hydrophobic face to each binding unit may permit the joining of the two binding units via the interaction between the hydrophobic interaction of the two molecules. In some embodiments, the linker may provide for polar interactions. For example, a leucine zipper domain of the proto-oncoproteins Myc and Max, respectively, may be used. Luscher and Larsson, Ongogene 18:2955-2966 (1999). In some embodiments, the linker allows for the formation of a salt bridge or disulfide bond. Linkers may comprise non-naturally occurring amino acids, as well as naturally occurring amino acids that are not naturally incorporated into a polypeptide. In some embodiments, the linker comprises a coordination complex between a metal or other ion and various residues from the multiple peptides joined thereby.

Linear peptide linkers of at least one amino acid residue are contemplated. In some embodiments the linker has more than 10,000 residues. In some embodiments the linker has from 1-10,000 residues. In some embodiments, the linker has from 1-1000 residues. In some embodiments, the linker has from 1-100 residues. In some embodiments, the linker has from 1-50 residues. In some embodiments the linker has 1-10 residues. In some embodiments, the linear peptide linker comprises residues with relatively inert side chains. Peptide linker amino acid residues need not be linked entirely or at all via alpha-carboxy and alpha-amino groups. That is, peptides may be linked via side chain groups of various residues.

The linker may affect whether the polypeptide(s) to which it is fused to is able to dimerize to each other or to another polypeptide. The linker serves a number of functions. Native receptor monomers restrained to the roughly two-dimensional plane of the cell membrane enjoy a relatively high local concentration and in the availability of co-receptors (binding units), increasing the probability of finding a partner. Receptors free in solution lacking such advantages may be aided by a linker that increases the effective concentration of the monomers.

In some embodiments, a binding construct may comprise more than one type of linker. Suitable linkers may also comprise the chemical modifications discussed below.

### C. Substituents And Other Chemical Modifications

The binding constructs of the invention may be chemically modified with various substituents. Such modifications preferably does not substantially reduce the growth factor binding affinities or specificities of the binding construct. Rather, the chemical modifications impart additional desirable characteristics as discussed herein. Chemical modifications may take a number of different forms such as heterologous peptides, polysaccarides, lipids, radioisotopes, non-standard amino acid resides and nucleic acids, metal chelates, and various toxins.

The receptor fragments, binding constructs, and other peptide molecules of the present invention may be fused to heterologous peptides to confer various properties, e.g., increased solubility, modulation of clearance, targeting to particular cell or tissue types. In some embodiments, the receptor fragment is linked to a Fc domain of IgG or other immunoglobulin. In some embodiments, a receptor fragment is fused to alkaline phosphatase (AP). Methods for making Fc or AP fusion constructs are found in WO 02/060950. By fusing the ligand binding domain of VEGFR-2 or VEGFR-3 (or other receptors) with protein domains that have specific properties (e.g. half life, bioavailability, interaction partners) it is possible to confer these properties to the VEGFR binding domains (e.g., the receptor binding domain could be engineered to have a specific tissue distribution or specific biological half life). In some embodiments, binding construct may include a co-receptor and a VEGFR fragment.

The particular heterologous polypeptide used in a particular construct can influence whether or not a growth factor receptor fragment will dimerize, which in turn may affect ligand binding. Fc fusion all may permit dimers, whereas AP fusions may permit monomers, cited, which along with Ig-domain boundary differences as possible reasons for different results obtained by different groups for receptor fragments binging to ligands. [Lu, et al., J. Biol. Chem. 275(19): 14321-14330 (2000).]

For substituents such as an Fc region of human IgG, the fusion can be fused directly to a binding construct or fused through an intervening sequence. For example, a human IgG hinge, CH2 and CH3 region may be fused at either the N-terminus or C-terminus of a binding construct to attach the Fc region. The resulting Fc-fusion construct enables purification via a Protein A affinity column (Pierce, Rockford, Ill.). Peptide and proteins fused to an Fc region can exhibit a substantially greater half-life in vivo than the unfused counterpart. A fusion to an Fc region allows for dimerization/multimerization of the fusion polypeptide. The Fc region may be a naturally occurring Fc region, or may be modified for superior characteristics, e.g., therapeutic qualities, circulation time, reduced aggregation.

Polypeptides can be modified, for instance, by glycosylation, amidation, carboxylation, or phosphorylation, or by the creation of acid addition salts, amides, esters, in particular C-terminal esters, and N-acyl derivatives. The proteins also can be modified to create peptide derivatives by forming covalent or noncovalent complexes with other moieties. Covalently bound complexes can be prepared by linking the chemical moieties to functional groups on the side chains of amino acids comprising the peptides, or at the N- or C-terminus.

Polypeptides can be conjugated to a reporter group, including, but not limited to a radiolabel, a fluorescent label, an enzyme (e.g., that catalyzes a calorimetric or fluorometric reaction), a substrate, a solid matrix, or a carrier (e.g., biotin or avidin). Examples of analogs are described in WO 98/28621 and in Olofsson, et al., Proc. Nat'l. Acad. Sci. USA, 95:11709-11714 (1998), U.S. Pat. Nos. 5,512,545, and 5,474,982; U.S. Patent Application Nos. 20020164687 and 20020164710.

Cysteinyl residues most commonly are reacted with haloacetates (and corresponding amines), such as chloroacetic acid or chloroacetamide, to give carboxymethyl or carbocyamidomethyl derivatives. Cysteinyl residues also are derivatized by reaction with bromotrifluoroacetone, α-bromo-β(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, orchloro-7-nitrobenzo-2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1M sodium cacodylate at pH 6.0.

Lysinyl and amino terminal residues are reacted with succinic or carboxylic acid anhydrides. Derivatization with these agents has the effect of reversing the charge of the lysinyl residues. Other suitable reagents for derivatizing α-amino-containing residues include imidoesters such as methyl picolinimidate; pyridoxal phosphate; pyridoxal; chloroborohydride; trinitrobenzenesulfonic acid; O-methylissurea; 2,4 pentanedione; and transaminase catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pK of the guanidine functional group. Furthermore, these reagents may react with the groups of lysine as well as the arginine epsilon-amino group.

The specific modification of tyrosyl residues per se has been studied extensively, with particular interest in introducing spectral labels into tyrosyl residues by reaction with aromatic diazonium compounds or tetranitromethane. Most commonly, N-acetylimidizol and tetranitromethane are used to form O-acetyl tyrosyl species and 3-nitro derivatives, respectively. Tyrosyl residues are iodinated using 1251 or 1311 to prepare labeled proteins for use in radioimmunoassay.

Carboxyl side groups (aspartyl or glutamyl) are selectively modified by reaction with carbodiimides (R1) such as 1-cyclohexyl-3-(2-morpholinyl-(4-ethyl) carbodiimide or 1-ethyl-3 (4 azonia 4,4-dimethylpentyl)carbodiimide. Furthermore, aspartyl and glutamyl residues are converted to asparaginyl and glutaminyl residues by reaction with ammonium ions.

Derivatization with bifunctional agents is useful for crosslinking the binding construct to water-insoluble support matrixes. Such derivation may also provide the linker that may connect adjacent binding elements in a binding construct, or a binding elements to a heterologous peptide, e.g., a Fc fragment. Commonly used crosslinking agents include, e.g., 1,1-bis(diazoacetyl)-2-phenylethane, glutaraldehyde, N-hydroxysuccinimide esters, for example, esters with 4-azidosalicylic acid, homo-bifunctional imidoesters, including disuccinimidyl esters such as 3,3′-dithiiobis(succinimidylpropioonate), and bifunctional maleimides such as bis-N-maleimido-1,8-octane. Derivatizing agents such as methyl-3-[(p-azidophenyl) dithio]propioimidate yield photoactivatable intermediates that are capable of forming cross links in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440, incorporated herein by reference, are employed for protein immobilization.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecule Properties, W. H. Freeman & Co., San Francisco, pp. 79-86, 1983), acetylation of the N-terminal amine, and, in some instances, amidation of the C-terminal carboxyl groups. Such derivatives are chemically modified polypeptide compositions in which the binding construct polypeptide is linked to a polymer. The polymer selected is typically water soluble so that the protein to which it is attached does not precipitate in an aqueous environment, such as a physiological environment. The polymer selected is usually modified to have a single reactive group, such as an active ester for acylation or an aldehyde for alkylation, so that the degree of polymerization may be controlled as provided for in the present methods. The polymer may be of any molecular weight, and may be branched or unbranched. Included within the scope of the binding construct polypeptide polymers is a mixture of polymers. Preferably, for therapeutic use of the end-product preparation, the polymer will be pharmaceutically acceptable.

The polymers each may be of any molecular weight and may be branched or unbranched. The polymers each typically have an average molecular weight of between about 2 kDa to about 100 kDa (the term “about” indicating that in preparations of a water soluble polymer, some molecules will weigh more, some less, than the stated molecular weight). The average molecular weight of each polymer is between about 5 kDa and about 50 kDa, more preferably between about 12 kDa to about 40 kDa and most preferably between about 20 kDa to about 35 kDa.

Suitable water soluble polymers or mixtures thereof include, but are not limited to, N-linked or 0-linked carbohydrates, sugars, phosphates, carbohydrates; sugars; phosphates; polyethylene glycol (PEG) (including the forms of PEG that have been used to derivatize proteins, including mono-(C1-C10) alkoxy- or aryloxy-polyethylene glycol); monomethoxy-polyethylene glycol; dextran (such as low molecular weight dextran, of, for example about 6 kD), cellulose; cellulose; other carbohydrate-based polymers, poly-(N-vinyl pyrrolidone)polyethylene glycol, propylene glycol homopolymers, a polypropylene oxide/ethylene oxide co-polymer, polyoxyethylated polyols (e.g., glycerol) and polyvinyl alcohol. Also encompassed by the present invention are bifunctional crosslinking molecules which may be used to prepare covalently attached multimers.

In general, chemical derivatization may be performed under any suitable condition used to react a protein with an activated polymer molecule. Methods for preparing chemical derivatives of polypeptides will generally comprise the steps of (a) reacting the polypeptide with the activated polymer molecule (such as a reactive ester or aldehyde derivative of the polymer molecule) under conditions whereby the binding construct becomes attached to one or more polymer molecules, and (b) obtaining the reaction product(s). The optimal reaction conditions will be determined based on known parameters and the desired result. For example, the larger the ratio of polymer molecules:protein, the greater the amount of attached polymer molecule. In one embodiment, the binding construct polypeptide derivative may have a single polymer molecule moiety at the amino terminus. (See, e.g., U.S. Pat. No. 5,234,784).

A particularly preferred water-soluble polymer for use herein is polyethylene glycol (PEG). As used herein, polyethylene glycol is meant to encompass any of the forms of PEG that can be used to derivatize other proteins, such as mono-(C1-C10) alkoxy- or aryloxy-polyethylene glycol. PEG is a linear or branched neutral polyether, available in a broad range of molecular weights, and is soluble in water and most organic solvents. PEG is effective at excluding other polymers or peptides when present in water, primarily through its high dynamic chain mobility and hydrophibic nature, thus creating a water shell or hydration sphere when attached to other proteins or polymer surfaces. PEG is nontoxic, non-immunogenic, and approved by the Food and Drug Administration for internal consumption.

Proteins or enzymes when conjugated to PEG have demonstrated bioactivity, non-antigenic properties, and decreased clearance rates when administered in animals. F. M. Veronese et al., Preparation and Properties of Monomethoxypoly(ethylene glycol)-modified Enzymes for Therapeutic Applications, in J. M. Harris ed., Poly(Ethylene Glycol) Chemistry—Biotechnical and Biomedical Applications, 127-36, 1992, incorporated herein by reference. These phenomena are due to the exclusion properties of PEG in preventing recognition by the immune system. In addition, PEG has been widely used in surface modification procedures to decrease protein adsorption and improve blood compatibility. S. W. Kim et al., Ann. N.Y. Acad. Sci. 516: 116-30 1987; Jacobs et al., Artif Organs 12: 500-501, 1988; Park et al., J. Poly. Sci, Part A 29:1725-31, 1991, incorporated herein by reference. Hydrophobic polymer surfaces, such as polyurethanes and polystyrene can be modified by the grafting of PEG (MW 3,400) and employed as nonthrombogenic surfaces. Surface properties (contact angle) can be more consistent with hydrophilic surfaces, due to the hydrating effect of PEG. More importantly, protein (albumin and other plasma proteins) adsorption can be greatly reduced, resulting from the high chain motility, hydration sphere, and protein exclusion properties of PEG.

PEG (MW 3,400) was determined as an optimal size in surface immobilization studies, Park et al., J. Biomed. Mat. Res. 26:739-45, 1992, while PEG (MW 5,000) was most beneficial in decreasing protein antigenicity. (F. M. Veronese et al., In J. M. Harris, et al., Poly(Ethylene Glycol) Chemistry—Biotechnical and Biomedical Applications, 127-36.)

Methods for preparing pegylated binding construct polypeptides will generally comprise the steps of (a) reacting the polypeptide with polyethylene glycol (such as a reactive ester or aldehyde derivative of PEG) under conditions whereby the binding construct polypeptide becomes attached to one or more PEG groups, and (b) obtaining the reaction product(s). In general, the optimal reaction conditions for the acylation reactions will be determined based on known parameters and the desired result. For example, the larger the ratio of PEG: protein, the greater the percentage of poly-pegylated product. In some embodiments, the binding construct will have a single PEG moiety at the N-terminus. See U.S. Pat. No. 8,234,784, herein incorporated by reference.

Derivatized binding constructs disclosed herein may have additional activities, enhanced or reduced biological activity, or other characteristics, such as increased or decreased half-life, as compared to the non-derivatized molecules.

### II. Polynucleotides Encoding Binding Constructs and Expression Systems

The invention comprises not only the binding constructs, binding units, and polypeptides described herein, and uses thereof, but also nucleic acids encoding such molecules, vectors comprising such molecules, and host cells comprising such vectors, and uses thereof. Methods employing any of the constructs, units, polypeptides, nucleic acids, vectors, and hosts cells for therapeutic uses described herein are all considered aspects of the invention.

### A. Nucleic Acids of the Invention

This invention also includes nucleic acid molecules whose sequence encode the polypeptides, binding units, and binding constructs, for use in compositions and methods of the invention. Nucleic acid molecules include those molecules which comprise nucleotide sequences which hybridize under moderately or highly stringent conditions as defined herein with the fully complementary sequence of the nucleic acid molecule of receptor tyrosine kinases described in Table 1A, or of a molecule encoding a polypeptide, which polypeptide comprises the receptor tyrosine kinase amino acids sequences described in Table 1A, or of a nucleic acid fragment as defined herein, or of a nucleic acid fragment encoding a polypeptide as defined herein.

Hybridization probes may be prepared using the sequences provided herein to screen cDNA, genomic or synthetic DNA libraries for related sequences. Regions of the DNA and/or amino acid sequence that exhibit significant identity to known sequences are readily determined using sequence alignment algorithms as described herein, and those regions may be used to design probes for screening.

The term “highly stringent conditions” refers to those conditions that are designed to permit hybridization of DNA strands whose sequences are highly complementary, and to exclude hybridization of significantly mismatched DNAs. Hybridization stringency is principally determined by temperature, ionic strength, and the concentration of denaturing agents such as formamide. Examples of “highly stringent conditions” for hybridization and washing are 0.015 M sodium chloride, 0.0015 M sodium citrate at 65-68° C. or 0.015 M sodium chloride, 0.0015 M sodium citrate, and 50% formamide at 42° C. See Sambrook, Fritsch & Maniatis, Molecular Cloning: A Laboratory Manual, 2nd Ed., Cold Spring Harbor Laboratory, (Cold Spring Harbor, N.Y. 1989); and Anderson et al., Nucleic Acid Hybridization: a Practical approach, Ch. 4, IRL Press Limited (Oxford, England). Limited, Oxford, England. Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate (NaDodSO₄ or SDS), ficoll, Denhardt's solution, sonicated salmon sperm DNA (or another non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, 6.8-7.4; however, at typical ionic strength conditions, the rate of hybridization is nearly independent of pH. See Anderson et al., Nucleic Acid Hybridization: a Practical Approach, Ch. 4, IRL Press Limited (Oxford, England).

Factors affecting the stability of a DNA duplex include base composition, length, and degree of base pair mismatch. Hybridization conditions can be adjusted by one skilled in the art in order to accommodate these variables and allow DNAs of different sequence relatedness to form hybrids. The melting temperature of a perfectly matched DNA duplex can be estimated by the following equation:

$${T}_{m} ( °C ) = 81.5 + 16.6 ( log [ {Na}^{+} ] ) + 0.41 ( \% G + C ) - 600 / N - 0.72 ( \% formamide )$$

where N is the length of the duplex formed, [Na⁺] is the molar concentration of the sodium ion in the hybridization or washing solution, % G+C is the percentage of (guanine+cytosine) bases in the hybrid. For imperfectly matched hybrids, the melting temperature is reduced by approximately 1° C. for each 1% mismatch.

The term “moderately” stringent conditions” refers to conditions under which a DNA duplex with a greater degree of base pair mismatching than could occur under “highly stringent conditions” is able to form. Examples of typical “moderately stringent conditions” are 0.015 M sodium chloride, 0.0015 M sodium citrate at 50-65° C. or 0.015 M sodium chloride, 0.0015 M sodium citrate, and 20% formamide at 37-50° C. By way of example, a “moderately stringent” condition of 50° C. in 0.015 M sodium ion will allow about a 21% mismatch.

It will be appreciated by those skilled in the art that there is no absolute distinction between “highly” and “moderately” stringent conditions. For example, at 0.015M sodium ion (no formamide), the melting temperature of perfectly matched long DNA is about 71° C. With a wash at 65° C. (at the same ionic strength), this would allow for approximately a 6% mismatch. To capture more distantly related sequences, one skilled in the art can simply lower the temperature or raise the ionic strength.

A good estimate of the melting temperature in 1M NaCl* for oligonucleotide probes up to about 20 nt is given by:

$$Tm = 2 °C per A - T base pair + 4 °C per G - C base pair$$

*The sodium ion concentration in 6× salt sodium citrate (SSC) is 1 M. See Suggs et al., Developmental Biology Using Purified Genes, p. 683, Brown and Fox (eds.) (1981).

High stringency washing conditions for oligonucleotides are usually at a temperature of 0-5° C. below the Tm of the oligonucleotide in 6×SSC, 0.1% SDS.

Differences in the nucleic acid sequence may result in conservative and/or non-conservative modifications of the amino acid sequence relative to the amino acid sequence. The invention is also directed to an isolated and/or purified DNA that corresponds to, or that hybridizes under stringent conditions with, any one of the foregoing DNA sequences.

### B. Preparation of DNA Encoding Ligand, Receptor, and Binding Construct Polypeptides

A nucleic acid molecule encoding all or part of a polypeptide of the invention such as a binding construct or binding unit of the invention can be made in a variety of ways, including, without limitation, chemical synthesis, cDNA or genomic library screening, expression library screening, and/or PCR amplification of cDNA or genomic DNA. These methods and others useful for isolating such DNA are set forth, for example, by Sambrook, et al., “Molecular Cloning: A Laboratory Manual,” Cold Spring Harbor Laboratory Press, Cold Spring Harbor, N.Y. (1989), by Ausubel, et al., eds., “Current Protocols In Molecular Biology,” Current Protocols Press (1994), and by Berger and Kimmel, “Methods In Enzymology: Guide To Molecular Cloning Techniques,” vol. 152, Academic Press, Inc., San Diego, Calif. (1987). Preferred nucleic acid sequences are mammalian sequences, such as human, rat, and mouse.

Chemical synthesis of nucleic acid molecules can be accomplished using methods well known in the art, such as those set forth by Engels, et al., Angew. Chem. Intl. Ed., 28:716-734 (1989). These methods include, inter alia, the phosphotriester, phosphoramidite and H-phosphonate methods of nucleic acid synthesis. Nucleic acids larger than about 100 nucleotides in length can be synthesized as several fragments, each fragment being up to about 100 nucleotides in length. The fragments can then be ligated together, as described below, to form the full length nucleic acid of interest. A preferred method is polymer-supported synthesis using standard phosphoramidite chemistry.

### C. Preparation of a Vector for Expression

The term “vector” refers to a nucleic acid molecule amplification, replication, and/or expression vehicle, often derived from or in the form of a plasmid or viral DNA or RNA system, where the plasmid or viral DNA or RNA is functional in a selected host cell, such as bacterial, yeast, plant, invertebrate, and/or mammalian host cells. The vector may remain independent of host cell genomic DNA or may integrate in whole or in part with the genomic DNA. The vector will contain all necessary elements so as to be functional in any host cell it is compatible with. Such elements are set forth below.

Nucleic acid encoding a polypeptide or fragment thereof has been isolated, it is preferably inserted into an amplification and/or expression vector in order to increase the copy number of the gene and/or to express the encoded polypeptide in a suitable host cell and/or to transform cells in a target organism (to express the polypeptide in vivo). Numerous commercially available vectors are suitable, though “custom made” vectors may be used as well. The vector is selected to be functional in a particular host cell or host tissue (i.e., for replication and/or expression). The polypeptide or fragment thereof may be amplified/expressed in prokaryotic and/or eukaryotic host cells, e.g., yeast, insect (baculovirus systems), plant, and mammalian cells. Selection of the host cell will depend at least in part on whether the polypeptide or fragment thereof is to be glycosylated. If so, yeast, insect, or mammalian host cells are preferable; yeast and mammalian cells will glycosylate the polypeptide if a glycosylation site is present on the amino acid sequence.

Typically, the vectors used in any of the host cells will contain 5′ flanking sequence and other regulatory elements such as an enhancer(s), a promoter, an origin of replication element, a transcriptional termination element, a complete intron sequence containing a donor and acceptor splice site, a signal peptide sequence, a ribosome binding site element, a polyadenylation sequence, a polylinker region for inserting the nucleic acid encoding the polypeptide to be expressed, and a selectable marker element. Optionally, the vector may contain a “tag” sequence, i.e., an oligonucleotide sequence located at the 5′ or 3′ end of the coding sequence that encodes polyHis (such as hexaHis) or another small immunogenic sequence. This tag will be expressed along with the protein, and can serve as an affinity tag for purification of the polypeptide from the host cell. Optionally, the tag can subsequently be removed from the purified polypeptide by various means such as using a selected peptidase.

The vector/expression construct may optionally contain elements such as a 5′ flanking sequence, an origin of replication, a transcription termination sequence, a selectable marker sequence, a ribosome binding site, a signal sequence, and one or more intron sequences. The 5′ flanking sequence may be homologous (i.e., from the same species and/or strain as the host cell), heterologous (i.e., from a species other than the host cell species or strain), hybrid (i.e., a combination of 5′ flanking sequences from more than one source), synthetic, or it may be the native polypeptide 5′ flanking sequence. As such, the source of the 5′ flanking sequence may be any unicellular prokaryotic or eukaryotic organism, any vertebrate or invertebrate organism, or any plant, provided that the 5′ flanking sequence is functional in, and can be activated by, the host cell machinery.

A transcription termination element is typically located 3′ to the end of the polypeptide coding sequence and serves to terminate transcription of the polypeptide. Usually, the transcription termination element in prokaryotic cells is a G-C rich fragment followed by a poly T sequence. Such elements can be cloned from a library, purchased commercially as part of a vector, and readily synthesized.

Selectable marker genes encode proteins necessary for the survival and growth of a host cell in a selective culture medium. Typical selectable marker genes encode proteins that (a) confer resistance to antibiotics or other toxins, e.g., ampicillin, tetracycline, or kanamycin for prokaryotic host cells, (b) complement auxotrophic deficiencies of the cell; or (c) supply critical nutrients not available from complex media.

A ribosome binding element, commonly called the Shine-Dalgarno sequence (prokaryotes) or the Kozak sequence (eukaryotes), is necessary for translation initiation of mRNA. The element is typically located 3′ to the promoter and 5′ to the coding sequence of the polypeptide to be synthesized. The Shine-Dalgarno sequence is varied but is typically a polypurine (i.e., having a high A-G content). Many Shine-Dalgarno sequences have been identified, each of which can be readily synthesized using methods set forth above.

All of the elements set forth above, as well as others useful in this invention, are well known to the skilled artisan and are described, for example, in Sambrook, et al., “Molecular Cloning: A Laboratory Manual,” Cold Spring Harbor Laboratory Press, Cold Spring Harbor, N.Y. (1989) and Berger, et al., eds., “Guide To Molecular Cloning Techniques,” Academic Press, Inc., San Diego, Calif. (1987].

For those embodiments of the invention where the recombinant polypeptide is to be secreted, a signal sequence is preferably included to direct secretion from the cell where it is synthesized. Typically, the polynucleotide encoding the signal sequence is positioned at the 5′ end of the coding region. Many signal sequences have been identified, and any of them that are functional in a target cell or species may be used in conjunction with the transgene.

In many cases, gene transcription is increased by the presence of one or more introns on the vector. The intron may be naturally-occurring, especially where the transgene is a full length or a fragment of a genomic DNA sequence. The intron may be homologous or heterologous to the transgene and/or to the transgenic mammal into which the gene will be inserted. The position of the intron with respect to the promoter and the transgene is important, as the intron must be transcribed to be effective. A preferred position for an intron is 3′ to the transcription start site, and 5′ to the polyA transcription termination sequence. For cDNA transgenes, an intron is placed on one side or the other (i.e., 5′ or 3′) of the transgene coding sequence. Any intron from any source, including any viral, prokaryotic and eukaryotic (plant or animal) organisms, may be used to express the polypeptide, provided that it is compatible with the host cell(s) into which it is inserted. Also included herein are synthetic introns. Optionally, more than one intron may be used in the vector.

Preferred vectors for recombinant expression are those that are compatible with bacterial, insect, and mammalian host cells. Such vectors include, inter alia, pCRII (Invitrogen Company, San Diego, Calif.), pBSII (Stratagene Company, La Jolla, Calif.), and pETL (BlueBacII; Invitrogen).

After the vector has been constructed and a nucleic acid has been inserted into the proper site of the vector, the completed vector may be inserted into a suitable host cell for amplification and/or polypeptide expression. Commonly used include: Prokaryotic cells such as gram negative or gram positive bacteria, i.e., any strain of E. coli, Bacillus, Streptomyces, Saccharomyces, Salmonella, and the like; eukaryotic cells such as CHO (Chinese hamster ovary) cells; human kidney 293 cells; COS-7 cells; insect cells such as Sf4, Sf5, Sf9, and Sf21 and High 5 (all from the Invitrogen Company, San Diego, Calif.); plant cells and various yeast cells such as Saccharomyces and Pichia. Any transformable or transfectable cell or cell line derived from any organism such as bacteria, yeast, fungi, monocot and dicot plants, plant cells, and animals are suitable.

Insertion (also referred to as “transformation” or “transfection”) of the vector into the selected host cell may be accomplished using such methods as calcium chloride, electroporation, microinjection, lipofection or the DEAE-dextran method. The method selected will in part be a function of the type of host cell to be used. These methods and other suitable methods are well known to the skilled artisan, and are set forth, for example, in Sambrook, et al., supra.

The host cells containing the vector (i.e., transformed or transfected) may be cultured using standard media well known to the skilled artisan. The media will usually contain all nutrients necessary for the growth and survival of the cells. Suitable media for culturing E. coli cells are for example, Luria Broth (LB) and/or Terrific Broth (TB). Suitable media for culturing eukaryotic cells are RPMI 1640, MEM, DMEM, all of which may be supplemented with serum and/or growth factors as required by the particular cell line being cultured. A suitable medium for insect cultures is Grace's medium supplemented with yeastolate, lactalbumin hydrolysate, and/or fetal calf serum as necessary.

Typically, an antibiotic or other compound useful for selective growth of the transformed cells only is added as a supplement to the media. The compound to be used will be dictated by the selectable marker element present on the plasmid with which the host cell was transformed. For example, where the selectable marker element is kanamycin resistance, the compound added to the culture medium will be kanamycin.

The amount of polypeptide produced in the host cell can be evaluated using standard methods known in the art. Such methods include, without limitation, Western blot analysis, SDS-polyacrylamide gel electrophoresis, non-denaturing gel electrophoresis, HPLC separation, immunoprecipitation, and/or binding assays.

### D. Purification of Polypeptides

If the polypeptide has been designed to be secreted from the host cells, the majority of polypeptide will likely be found in the cell culture medium. If, however, the polypeptide is not secreted from the host cells, it will be present in the cytoplasm (for eukaryotic, gram-positive bacteria, and insect host cells) or in the periplasm (for gram negative bacteria host cells).

For intracellular polypeptides, the host cells are first disrupted mechanically or osmotically to release the cytoplasmic contents into a buffered solution. The polypeptide is then isolated from this solution.

Purification of the polypeptide from solution can be accomplished using a variety of techniques. If the polypeptide has been synthesized such that it contains a tag such as hexahistidine or other small peptide at either its carboxyl or amino terminus, it may essentially be purified in a one-step process by passing the solution through an affinity column where the column matrix has a high affinity for the tag or for the polypeptide directly (i.e., a monoclonal antibody specifically recognizing the polypeptide). For example, polyhistidine binds with great affinity and specificity to nickel, thus an affinity column of nickel (such as the Qiagen nickel columns) can be used for purification of the His-tagged polypeptide. (See, for example, Ausubel, et al., eds., “Current Protocols In Molecular Biology,” Section 10.11.8, John Wiley & Sons, New York (1993)).

The strong affinity a ligand for its receptor permits affinity purification of binding constructs, and binding constructs using an affinity matrix comprising a complementary binding partner. Affinity chromatography may be employed, e.g., using either natural binding partners (e.g., a ligand when purifying a binding construct with affinity for the same) or antibodies generated using standard procedures (e.g., immunizing a mouse, rabbit or other animal with an appropriate polypeptide). The peptides of the present invention may be used to generate such antibodies. Known antibodies or antibodies to known growth factor receptors may be employed when they share an epitope with a targeted binding construct.

In addition, other well known procedures for purification can be used. Such procedures include, without limitation, ion exchange chromatography, molecular sieve chromatography, HPLC, native gel electrophoresis in combination with gel elution, and preparative isoelectric focusing (“Isoprime” machine/technique, Hoefer Scientific). In some cases, two or more of these techniques may be combined to achieve increased purity. Preferred methods for purification include polyhistidine tagging and ion exchange chromatography in combination with preparative isoelectric focusing.

Polypeptide found in the periplasmic space of the bacteria or the cytoplasm of eukaryotic cells, the contents of the periplasm or cytoplasm, including inclusion bodies (bacteria) if the processed polypeptide has formed such complexes, can be extracted from the host cell using any standard technique known to the skilled artisan. For example, the host cells can be lysed to release the contents of the periplasm by French press, homogenization, and/or sonication. The homogenate can then be centrifuged.

If the polypeptide has formed inclusion bodies in the periplasm, the inclusion bodies can often bind to the inner and/or outer cellular membranes and thus will be found primarily in the pellet material after centrifugation. The pellet material can then be treated with a chaotropic agent such as guanidine or urea to release, break apart, and solubilize the inclusion bodies. The solubilized polypeptide can then be analyzed using gel electrophoresis, immunoprecipitation or the like. If it is desired to isolate the polypeptide, isolation may be accomplished using standard methods such as those set forth below and in [Marston, et al., Meth. Enz., 182:264-275 (1990).]

### III. Anti-Ligand and Anti-Receptor Therapeutic Compounds

Anti-ligand or anti-receptor therapies as discussed below include, but are not limited to antibody, aptamer, antisense and interference RNA techniques and therapies.

Exemplary anti-VEGFR-3 antibodies and their production are described in U.S. Pat. Nos. 6,107,046 and 6,824,777; U.S. Patent Publication Nos. 2006/0269548 and 2006/0177901; and International Patent Application No. PCT/F195/00337 (WO 95/33772), all incorporated herein by reference in their entireties.

Exemplary VEGF-D antibodies are described, for example, in International Patent Application Nos. PCT/US97/14696 and PCT/US99/31332; International Publication No.: WO 0037025; U.S. Pat. Nos. 6,383,484, 6,730,489 and 7,097,986; and U.S. Patent Publication Nos. 2006/0177428, 2006/0024302, 2002/0123481, 2005/0282228, 2004/0141917 and 2003/0125537, all incorporated herein by reference.

Exemplary VEGF-C antibodies are described, for example, in International Patent Application Nos. PCT/FI1996/000427 (WO/1997/005250) and PCT/US1998/001973 (WO/1998/033917); and U.S. Patent Publication Nos. 2004/0147726, 2005/0232921, 2005/0192429, 2005/0059117, 2005/0282228, 2003/0176674, and 2006/0121025, 2006/0030000, and U.S. Pat. No. 6,403,088 all incorporated herein by reference.

U.S. Pat. No. 7,045,133, incorporated herein by reference, describes peptidomimetic inhibitors of VEGF-D/VEGF-C/VEGFR-3.

Exemplary anti-PDGFR antibodies and other inhibitor compounds are described, for example, in U.S. Pat. Nos. 5,418,135; 5,468,468; 5,620,687; 5,932,580; 6,358,954; 6,642,022; and 7,105,305, all incorporated herein by reference.

Exemplary anti-VEGFR antibodies and other inhibitor compounds are described, for example, in U.S. Pat. Nos. 7,056,509; 7,052,693; 6,986,890; 6,897,294; 6,887,468; 6,878,720; 6,344,339; 5,955,311; 5,874,542; and 5,840,301, all incorporated herein by reference.

The following description makes specific reference to the production, testing, and use of particular anti-VEGFR-2 antibodies, as representative of the many receptor and growth factor and growth factor antigens described herein. The methods described may also be readily adapted for the production of other antibodies for use according to the present invention, e.g., anti-growth factor ligand antibodies and anti-receptor antibodies as binding units of the binding constructs. Such antibody-type binding units may themselves the used for practicing methods of the invention, or form one binding unit of a more complex, multivalent binding construct. In some embodiments a binding construct has at least one binding unit that comprising a receptor fragment and at least one binding unit that comprises an antigen binding fragment. Antibodies directed against growth factors and receptors may also be used in combination with the binding constructs of the invention. Exemplary antibodies may be found in U.S. patent application Ser. No. 11/075,400, published as U.S. Patent Publication No. 2005/0282233, and related, co-filed International Patent Application No. PCT/US2005/007742, published as WO 2005/087812 (Attorney Docket No. 28967/39820B); and U.S. Patent Publication Nos. 2006/0177428; 2006/0024302; 2004/0175730; and 2004/0141917; all applications are incorporated by reference in their entireties.

### A. Therapeutic Anti-VEGFR-2 Selective VEGF-A Antagonist Antibodies

Polyclonal or monoclonal therapeutic anti-VEGFR-2 antibodies useful in practicing this invention may be prepared in laboratory animals or by recombinant DNA techniques using the following methods. Polyclonal antibodies to the VEGFR-2 molecule or a fragment thereof containing the target amino acid sequence generally are raised in animals by multiple subcutaneous (sc) or intraperitoneal (ip) injections of the VEGFR-2 molecule in combination with an adjuvant such as Freund's adjuvant (complete or incomplete). To enhance immunogenicity, it may be useful to first conjugate the VEGFR-2 molecule or a fragment containing the target amino acid sequence of a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl, or R¹N═C═NR, where R and R¹ are different alkyl groups. Alternatively, VEGF-2-immunogenic conjugates can be produced recombinantly as fusion proteins.

Animals are immunized against the immunogenic VEGFR-2 conjugates or derivatives (such as a fragment containing the target amino acid sequence) by combining about 1 mg or about 1 microgram of conjugate (for rabbits or mice, respectively) with about 3 volumes of Freund's complete adjuvant and injecting the solution intradermally at multiple sites. Approximately 7 to 14 days later, animals are bled and the serum is assayed for anti-VEGFR-2 titer. Animals are boosted with antigen repeatedly until the titer plateaus. Preferably, the animal is boosted with the same VEGFR-2 molecule or fragment thereof as was used for the initial immunization, but conjugated to a different protein and/or through a different cross-linking agent. In addition, aggregating agents such as alum are used in the injections to enhance the immune response.

Monoclonal antibodies may be prepared by recovering spleen cells from immunized animals and immortalizing the cells in conventional fashion, e.g. by fusion with myeloma cells. The clones are then screened for those expressing the desired antibody. The monoclonal antibody preferably does not cross-react with other VEGFR family members.

Preparation of antibodies using recombinant DNA methods such as the phagemid display method, may be accomplished using commercially available kits, as for example, the Recombinant Phagemid Antibody System available from Pharmacia (Uppsala, Sweden), or the SurfZAP™ phage display system (Stratagene Inc., La Jolla, Calif.).

One may increase the population of anti-VEGFR-2 antibodies that selectively block VEGF-A binding by using a Ig-domain 3 or other fragment as the immunogen, but that is not necessary. After antibodies are generated, they may be tested to ascertain their specific affinities. Competition studies may be performed that show that the antibody competes for binding to VEGFR-2 with VEGF-A, but not with VEGF-C.

One method comprises incubating VEGFR-2 expressing cells with either labeled-VEGF-A alone, the antibody being tested alone, or with both the VEGF-A and the antibody. A label on the antibody may be employed in addition to that on VEGF-A or instead of that label. The antibody may also be detected using a labeled secondary antibody. The first two groups acting as controls allow one to confirm that both the antibody and the VEGF-A ligand (or optionally VEGF-E) are able to bind to the receptor in the absence of the other. Those cell samples treated with both VEGF-A (or VEGF-E) and an antibody, that reveal binding of the antibody, but not VEGF-A (or VEGF-E) indicate that the antibody should be further tested. As described below, stoichiometric analysis can be used to ascertain that the ligand and antibody are competing for the same molecule.

This further testing may comprise binding studies that reveal that both VEGF-C(or VEGF-D) and the antibody are able to bind the receptor simultaneously. This testing also is designed to determine whether VEGF-C and the antibody are simultaneously binding to a single VEGFR-2 molecule as opposed to binding of VEGF-C and the antibody binding to different VEGFR-2 molecules. Comparative quantitative binding studies may accordingly be used. The VEGFR-2 cells are counted in each sample. VEGFR-2 samples, having been counted, are incubated with either labeled VEGF-C alone or labeled (or unlabeled using a secondary antibody for detection) antibody alone. The degree of binding is measured, quantitated, using suitable imaging procedures, e.g., if radiolabel is employed using a phosphoimager. The average number of VEGFR-2 receptors per cell are calculated by dividing the amount of bound molecules by the total number of cells. Whether the receptors are saturated with molecules may be achieved by repeating the assay with increasing amounts of the labeled molecule(s). The binding assay is repeated again with both ligand and antibody. If the quantification reveals that the number of antibodies and ligands bound is greater than the total number of receptors, then the antibody has the desired characteristics.

The described protocols may also be modified and used to produce antibodies against any of the other antigens identified herein as targets for anti-rejection therapy, including but not limited to VEGFR-3, VEGF-C, VEGF-D, the other VEGF growth factors, the PDGF receptors, and the PDGF growth factors.

Preferably, antibodies for administration to humans, although prepared in a laboratory animal such as a mouse, will be “humanized”, or chimeric, i.e. made to be compatible with the human immune system such that a human patient will not develop an immune response to the antibody. Even more preferably, human antibodies which can now be prepared using methods such as those described for example, in Lonberg, et al., Nature Genetics, 7:13-21 (1994) are preferred for therapeutic administration to patients. Fully human antibodies are highly preferred.

### 1. Humanization Of Anti-VEGFR-2 Monoclonal Antibodies

Selective binding agents, including monoclonal antibodies, which selectively block VEGF-A without blocking VEGF-C(or VEGF-D) binding may be applied therapeutically. Following are protocols to improve the utility of anti-VEGFR-2 monoclonal antibodies as therapeutics in humans, by “humanizing” the monoclonal antibodies to improve their serum half-life and render them less immunogenic in human hosts (i.e., to prevent human antibody response to non-human anti-VEGFR-2 antibodies). The description also applies to antibodies directed to the other antigens described herein.

The principles of humanization have been described in the literature and are facilitated by the modular arrangement of antibody proteins. To minimize the possibility of binding complement, a humanized antibody of the IgG4 isotype is preferred.

For example, a level of humanization is achieved by generating chimeric antibodies comprising the variable domains of non-human antibody proteins of interest, such as the anti-VEGFR-2 monoclonal antibodies described herein, with the constant domains of human antibody molecules. (See, e.g., Morrison and Oi, Adv. Immunol., 44:65-92 (1989).) The variable domains of VEGFR-2 neutralizing anti-VEGFR-2 antibodies are cloned from the genomic DNA of a B-cell hybridoma or from cDNA generated from mRNA isolated from the hybridoma of interest. The V region gene fragments are linked to exons encoding human antibody constant domains, and the resultant construct is expressed in suitable mammalian host cells (e.g., myeloma or CHO cells).

To achieve an even greater levels of humanization, only those portions of the variable region gene fragments that encode antigen-binding complementarity determining regions (“CDR”) of the non-human monoclonal antibody genes are cloned into human antibody sequences. [See, e.g., Jones et al., Nature, 321:522-525 (1986); Riechmann et al., Nature, 332:323-327 (1988); Verhoeyen et al., Science, 239:1534-36 (1988); and Tempest et al., Bio/Technology, 9:266-71 (1991).] If necessary, the B-sheet framework of the human antibody surrounding the CDR3 regions also is modified to more closely mirror the three-dimensional structure of the antigen-binding domain of the original monoclonal antibody. [(See Kettleborough et al., Protein Engin., 4:773-783 (1991); and Foote et al., J. Mol. Biol., 224:487-499 (1992).)]

In an alternative approach, the surface of a non-human monoclonal antibody of interest is humanized by altering selected surface residues of the non-human antibody, e.g., by site-directed mutagenesis, while retaining all of the interior and contacting residues of the non-human antibody. [See Padlan, Molecular Immunol., 28(4/5):489-98 (1991).]

The foregoing approaches are employed using VEGFR-2-neutralizing anti-VEGFR-2 monoclonal antibodies and the hybridomas that produce them to generate humanized VEGFR-2-neutralizing antibodies useful as therapeutics to treat or palliate conditions wherein VEGFR-2 expression is detrimental and/or activation by VEGF-A. One therapeutic target is selective promotion of lymphangiogenesis while minimizing promotion of angiogenesis.

### 2. Human VEGFR-2-Neutralizing Antibodies From Phage Display

Human VEGFR-2-neutralizing antibodies are generated by phage display techniques such as those described in Aujame et al., Human Antibodies, 8(4):155-168 (1997); Hoogenboom, TIBTECH, 15:62-70 (1997); and Rader et al., Curr. Opin. Biotechnol., 8:503-508 (1997), all of which are incorporated by reference. For example, antibody variable regions in the form of Fab fragments or linked single chain Fv fragments are fused to the amino terminus of filamentous phage minor coat protein pIII. Expression of the fusion protein and incorporation thereof into the mature phage coat results in phage particles that present an antibody on their surface and contain the genetic material encoding the antibody. A phage library comprising such constructs is expressed in bacteria, and the library is panned (screened) for VEGFR-2-specific phage-antibodies using labeled or immobilized VEGFR-2 as antigen-probe.

### 3. Human VEGFR-2-Neutralizing Antibodies From Transgenic Mice

Human VEGFR-2-neutralizing antibodies are generated in transgenic mice essentially as described in Bruggemann and Neuberger, Immunol. Today, 17(8):391-97 (1996) and Bruggemann and Taussig, Curr. Opin. Biotechnol., 8:455-58 (1997). Transgenic mice carrying human V-gene segments in germline configuration and that express these transgenes in their lymphoid tissue are immunized with an VEGFR-2 composition using conventional immunization protocols. Hybridomas are generated using B cells from the immunized mice using conventional protocols and screened to identify hybridomas secreting anti-VEGFR-2 human antibodies (e.g., as described above).

### 4. Bispecific Antibodies

Bispecific antibodies that specifically bind to VEGFR-2 and that specifically bind to other antigens relevant to pathology and/or treatment are produced, isolated, and tested using standard procedures that have been described in the literature. See, e.g., Pluckthun & Pack, Immunotechnology, 3:83-105 (1997); Carter et al., J. Hematotherapy, 4: 463-470 (1995); Renner & Pfreundschuh, Immunological Reviews, 1995, No. 145, pp. 179-209; Pfreundschuh U.S. Pat. No. 5,643,759; Segal et al., J. Hematotherapy, 4: 377-382 (1995); Segal et al., Immunobiology, 185: 390-402 (1992); and Bolhuis et al., Cancer Immunol. Immunother., 34: 1-8 (1991), all of which are incorporated herein by reference in their entireties. Bispecific antibodies that may be employed in combination with the binding constructs of the invention include those described in U.S. Patent Publication No. 2005/0282233, incorporated herein by reference.

For example, bispecific antibodies (bscAb) are produced by joining two single-chain Fv fragments via a glycine-serine linker using recombinant methods. The V light-chain (V_{L}) and V heavy-chain (V_{H}) domains of two antibodies of interest are isolated using standard PCR methods. The V_{L} and V_{H} cDNA's obtained from each hybridoma are then joined to form a single-chain fragment in a two-step fusion PCR. Bispecific fusion proteins are prepared in a similar manner. Bispecific single-chain antibodies and bispecific fusion proteins are antibody substances included within the scope of the present invention.

Antibody fragments that contain the antigen binding, or idiotype, of the molecule may be generated by known techniques. For example, such fragments include, but are not limited to, the F(ab′)₂ fragment which may be produced by pepsin digestion of the antibody molecule; the Fab′ fragments which may be generated by reducing the disulfide bridges of the F(ab′)₂ fragment, and the two Fab′ fragments which may be generated by treating the antibody molecule with papain and a reducing agent.

Chemically constructed bispecific antibodies may be prepared by chemically cross-linking heterologous Fab or F(ab′)₂ fragments by means of chemicals such as heterobifunctional reagent succinimidyl-3-(2-pyridyldithiol)-propionate (SPDP, Pierce Chemicals, Rockford, Ill.). The Fab and F(ab′)₂ fragments can be obtained from intact antibody by digesting it with papain or pepsin, respectively (Karpovsky et al., J. Exp. Med. 160:1686-701, 1984; Titus et al., J. Immunol., 138:4018-22, 1987).

### 5. Humanization of Known Anti-VEGFR-2 Antibodies

Existing anti-VEGF-2 antibodies may also be employed in the various methods and compositions of the present invention, and, if not already humanized, may be humanized as discussed herein. Known anti-VEGFR-2 antibodies may be tested for the ability to selectively block VEGF-A binding using the methods discussed herein. Known anti-VEGFR-2 antibodies (anti-KDR antibodies) are taught for example in Lu et al., J. Immunological Methods, 230:159-71 (1999); Lu, et al., J. Biol. Chem., 275(19): 14321-14330 (2000); and Lu, et al., J. Biol. Chem., 278(44): 43496-43507 (2003).

### 6. Domain Antibodies

A domain antibody comprises a functional binding unit of an antibody, and can correspond to the variable regions of either the heavy (V_{H}) or light (V_{L}) chains of antibodies. A domain antibody can have a molecular weight of approximately 13 kDa, or approximately one-tenth of a full antibody. Domain antibodies may be derived from full antibodies such as those described herein.

### B. Anti-Receptor And Anti-Ligand Aptamers

Recent advances in the field of combinatorial sciences have identified short polymer sequences with high affinity and specificity to a given target. For example, SELEX technology has been used to identify DNA and RNA aptamers with binding properties that rival mammalian antibodies, the field of immunology has generated and isolated antibodies or antibody fragments which bind to a myriad of compounds and phage display has been utilized to discover new peptide sequences with very favorable binding properties. Based on the success of these molecular evolution techniques, it is certain that molecules can be created which bind to any target molecule. A loop structure is often involved with providing the desired binding attributes as in the case of: aptamers which often utilize hairpin loops created from short regions without complimentary base pairing, naturally derived antibodies that utilize combinatorial arrangement of looped hyper-variable regions and new phage display libraries utilizing cyclic peptides that have shown improved results when compared to linear peptide phage display results. Thus, sufficient evidence has been generated to suggest that high affinity ligands can be created and identified by combinatorial molecular evolution techniques. For the present invention, molecular evolution techniques can be used to isolate binding constructs specific for ligands described herein. For more on aptamers, See generally, Gold, L., Singer, B., He, Y. Y., Brody. E., “Aptamers As Therapeutic And Diagnostic Agents,” J. Biotechnol. 74:5-13 (2000). Relevant techniques for generating aptamers may be found in U.S. Pat. No. 6,699,843, which is incorporated by reference in its entirety.

In some embodiments, the aptamer may be generated by preparing a library of nucleic acids; contacting the library of nucleic acids with a growth factor, wherein nucleic acids having greater binding affinity for the growth factor (relative to other library nucleic acids) are selected and amplified to yield a mixture of nucleic acids enriched for nucleic acids with relatively higher affinity and specificity for binding to the growth factor. The processes may be repeated, and the selected nucleic acids mutated and rescreened, whereby a growth factor aptamer is be identified. Nucleic acids may be screened to select for molecules that bind to more than growth factor. Binding more than one growth factor can refer to binding more than one growth factor simultaneously or competitively. In some embodiments a binding construct will comprise at least one aptamer, wherein a first binding unit binds VEGF-A and a second binding unit binds VEGF-C. In some embodiments a binding construct will comprise at least one aptamer, wherein a first binding unit binds a VEGF growth factor subfamily member and a second binding unit binds a PDGF subfamily member.

### C. Anti-Sense Molecules And Therapy

Another class of inhibitors that may be used in conjunction with the present invention is isolated antisense nucleic acid molecules that can hybridize to, or are complementary to, the nucleic acid molecule, nucleotide sequence, or fragments, analogs or derivatives thereof. Antisense modulation of VEGF-C is described in U.S. Patent Application Publication No. 2003/0232437, the disclosure of which is incorporated herein by reference in its entirety. Antisense modulation of VEGFR-2 is described in U.S. Pat. No. 6,734,017, the disclosure of which is incorporated herein by reference in its entirety.

Antisense and interfering RNA molecules that target any of the growth factors (e.g., VEGF-A, -B, -C, -D; PDGF-A, -B, -C, -D) and growth factor receptors (e.g., VEGFR-1, VEGFR-2, VEGFR-3, PDGFR-alpha, PDGFR-beta) described herein are specifically contemplated for use in methods and products of the invention.

An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific embodiments, antisense nucleic acid molecules are provided that comprise a sequence complementary to at least about 10, 25, 50, 100, 250 or 500 nucleotides or an entire receptor or ligand coding strand, or to only a portion thereof. Nucleic acid molecules encoding fragments, homologs, derivatives and analogs of receptor or ligand or antisense nucleic acids complementary to a receptor or ligand nucleic acid sequence are additionally provided.

In one embodiment, an antisense nucleic acid molecule is antisense to a “coding region” of the coding strand of a nucleotide sequence encoding a receptor or ligand protein (or fragments or fragment combination thereof). The term “coding region” refers to the region of the nucleotide sequence comprising codons that are translated into amino acid residues. In another embodiment, the antisense nucleic acid molecule is antisense to a “conceding region” of the coding strand of a nucleotide sequence encoding the receptor or ligand protein. The term “conceding region” refers to 5′ and 3′ sequences that flank the coding region and that are not translated into amino acids (i.e., also referred to as 5′ and 3′ untranslated regions).

Given the coding strand sequences encoding the receptor or ligand protein disclosed herein, antisense nucleic acids of the invention can be designed according to the rules of Watson and Crick or Hoogsteen base pairing. The antisense nucleic acid molecule can be complementary to the entire coding region of a ligand or receptor mRNA, but more preferably is an oligonucleotide that is antisense to only a portion of the coding or noncoding region of receptor or ligand mRNA. For example, the antisense oligonucleotide can be complementary to the region surrounding the translation start site of receptor or ligand mRNA. An antisense oligonucleotide can be, for example, about 5, 10, 15, 20, 25, 30, 35, 40, 45, or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following section).

The antisense nucleic acid molecules of the invention are typically administered to a subject or generated in situ such that they hybridize with or bind to cellular mRNA and/or genomic DNA encoding a receptor or ligand to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an alpha-anomeric nucleic acid molecule. An alpha-anomeric nucleic acid molecule forms specific double-stranded hybrids with complementary RNA in which, contrary to the usual alpha-units, the strands run parallel to each other. See, e.g., Gaultier, et al., Nucl. Acids Res., 15:6625-6641 (1987). The antisense nucleic acid molecule can also comprise a 2′-o-methylribonucleotide (see, e.g., Inoue, et al. Nucl. Acids Res., 15:6131-6148 (1987)) or a chimeric RNA-DNA analogue (see, e.g., Inoue, et al., FEBS Lett., 215:327-330 (1987)).

Production and delivery of antisense molecules are facilitated by providing a vector comprising an anti-sense nucleotide sequence complementary to at least a part of the Receptor or ligand DNA sequence. According to a yet further aspect of the invention such a vector comprising an anti-sense sequence may be used to inhibit, or at least mitigate, Receptor or ligand expression.

Alternatively, nucleic acid sequences which inhibit or interfere with gene expression (e.g., siRNA, shRNA, ribozymes, aptamers) can be used to inhibit or interfere with the activity of RNA or DNA encoding a target protein.

### D. Anti-Ligand or Anti-Receptor RNA Interference

Use of RNA Interference to inactivate or modulate receptor or ligand expression is also contemplated by this invention. RNA interference is described in U.S. Patent Appl. Pub. No. 2002/0162126, and Hannon, G., J. Nature, 11:418:244-51 (2002). “RNA interference,” “post-transcriptional gene silencing,” “quelling”-—these terms have all been used to describe similar effects that result from the overexpression or misexpression of transgenes, or from the deliberate introduction of double-stranded RNA into cells (reviewed in Fire, A., Trends Genet 15:358-363 (1999); Sharp, P. A., Genes Dev., 13:139-141 (1999); Hunter, C., Curr. Biol., 9:R440-R442 (1999); Baulcombe, D. C., Curr. Biol. 9:R599-R601 (1999); Vaucheret, et al. Plant J. 16:651-659 (1998), all incorporated by reference. RNA interference, commonly referred to as RNAi, offers a way of specifically and potently inactivating a cloned gene. RNA interference of the VEGF family of proteins and receptors is described in U.S. Patent application Publicaiton Nos.: 2006/0217332, 2006/0025370, 2005/0233998, 2005/0222066 and 2005/0171039, the disclosure of which are incorporated herein by reference in their entireties.

Interfering RNA directed to VEGF or VEGFR family members is described in U.S. Patent Publication No. 2006/0217332, incorporated herein by reference.

siRNA (short interfering RNA) technology relates to a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Accordingly, siRNA may be effected by introduction or expression of relatively short homologous dsRNAs. Indeed the use of relatively short homologous dsRNAs may have certain advantages as discussed below.

Compared to siRNA, shRNA (short hairpin RNA) offers advantages in silencing longevity and delivery options. See, Hannon et al., Nature, 431:371-378, 2004, for review. Vectors that produce shRNAs, which are processed intracellularly into short duplex RNAs having siRNA-like properties have been reported (Brummelkamp et al., Science 296, 550-553, 2000; Paddison et al., Genes Dev. 16, 948-958 (2002). Such vectors provide a renewable source of a gene-silencing reagent that can mediate persistent gene silencing after stable integration of the vector into the host-cell genome. Furthermore, the core silencing ‘hairpin’ cassette can be readily inserted into retroviral, lentiviral or adenoviral vectors, facilitating delivery of shRNAs into a broad range of cell types (Brummelkamp et al., Cancer Cell 2:243-247, 2002; Dirac, et al., J. Biol. Chem. 278:11731-11734, 2003; Michiels et al., Nat. Biotechnol. 20:1154-1157, 2002; Stegmeie et al., Proc. Natl. Acad. Sci. USA 102:13212-13217, 2005; Khvorova et al., Cell, 115:209-216 (2003) in any of the innumerable ways that have been devised for delivery of DNA constructs that allow ectopic mRNA expression. These include standard transient transfection, stable transfection and delivery using viruses ranging from retroviruses to adenoviruses. Expression can also be driven by either constitutive or inducible promoter systems (Paddison et al., Methods Mol. Biol. 265:85-100, 2004). Delivery of nucleic acid inhibitors by replicating or replication-deficient vectors is contemplated as an aspect of the invention.

Mammalian cells have at least two pathways that are affected by double-stranded RNA (dsRNA). In the siRNA (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length.

The nonspecific effects occur because dsRNA activates two enzymes: PKR, which in its active form phosphorylates the translation initiation factor eIF2 to shut down all protein synthesis, and 2′, 5′ oligoadenylate synthetase (2′, 5′-AS), which synthesizes a molecule that activates RNase L, a nonspecific enzyme that targets all mRNAs. The nonspecific pathway may represent a host response to stress or viral infection, and, in general, the effects of the nonspecific pathway are preferably minimized. Significantly, longer dsRNAs appear to be required to induce the nonspecific pathway and, accordingly, dsRNAs shorter than about 30 bases pairs are preferred to effect gene repression by RNAi (see Hunter et al., 1975, J. Biol. Chem. 250:409-17; Manche et al., 1992, Mol. Cell. Biol. 12:5239-48; Minks et al., 1979, J. Biol. Chem. 254:10180-3; and Elbashir et al., 2001, Nature 411:494-8). siRNA has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass, 2001, Nature 411:428-9). In mammalian cells, siRNAs are effective at concentrations that are several orders of magnitude below the concentrations typically used in antisense experiments (Elbashir et al., 2001, Nature 411:494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al., 2001, Nature 411:494-8).

Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernible to the skilled artisan.

Exemplary dsRNAs may be synthesized chemically or produced in vitro or in vivo using appropriate expression vectors. Exemplary synthetic RNAs include 21 nucleotide RNAs chemically synthesized using methods known in the art. Synthetic oligonucleotides are preferably deprotected and gel-purified using methods known in the art (see e.g. Elbashir et al., 2001, Genes Dev. 15:188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species will be designed to include a portion of nucleic acid sequence represented in a target nucleic acid.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference.

Although mRNAs are generally thought of as linear molecules containing the information for directing protein synthesis within the sequence of ribonucleotides, most mRNAs have been shown to contain a number of secondary and tertiary structures. Secondary structural elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g. Jaeger et al., 1989, Proc. Natl. Acad. Sci. USA 86:7706; and Turner et al.., 1988, Annu. Rev. Biophys. Biophys. Chem. 17:167). The rules are useful in identification of RNA structural elements and, in particular, for identifying single stranded RNA regions which may represent preferred segments of the mRNA to target for siRNA, ribozyme or antisense technologies. Accordingly, preferred segments of the mRNA target can be identified for design of the siRNA mediating dsRNA oligonucleotides as well as for design of appropriate ribozyme and hammerheadribozyme compositions of the invention (see below).

The dsRNA oligonucleotides may be introduced into the cell by transfection with a heterologous target gene using carrier compositions such as liposomes, which are known in the art—e.g. Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al., 1998, J. Cell Biol. 141:863-74). The effectiveness of the siRNA may be assessed by any of a number of assays following introduction of the dsRNAs. These include Western blot analysis using antibodies which recognize the target gene product following sufficient time for turnover of the endogenous pool after new protein synthesis is repressed, reverse transcriptase polymerase chain reaction and Northern blot analysis to determine the level of existing target mRNA.

Further compositions, methods and applications of siRNA technology are provided in U.S. Pat. Nos. 6,278,039, 5,723,750 and 5,244,805, which are incorporated herein by reference.

### E. Small Molecule Inhibitors

Any chemical substance that can be safely administered as a therapeutic and that can be used to modulate biochemical pathway targets identified herein, such as VEGF-mediated stimulation of VEGF receptors, may be used to practice the invention. Small molecules that inhibit the interaction between VEGF-C and/or VEGFR-3 with VEGFR-3 are specifically contemplated. VEGF-C/VEGF-D inhibitors are disclosed in U.S. Pat. No. 7,045,133, incorporated herein by reference.

The VEGF receptors are receptor tyrosine kinases and intracellular signaling is initiated through receptor phosphorylation. Accordingly, one preferred class of molecules for practice of the invention is tyrosine kinase inhibitors, including those described in and Morin, Oncogene, 19(56):6574-83, 2000, incorporated herein by reference. VEGFR-3 inhibitors are disclosed in U.S. Patent Publication No. 2002-0164667, incorporated herein by reference.

### IV. Therapeutic Formulations and Administration

### A. Therapeutic Formulations

Binding constructs, or polynucleotides encoding the same, can be used directly to practice materials and methods of the invention, but in preferred embodiments, the compounds are formulated with pharmaceutically acceptable diluents, adjuvants, excipients, or carriers. The phrase “pharmaceutically or pharmacologically acceptable” refers to molecular entities and compositions that do not produce adverse, allergic, or other untoward reactions when administered to an animal or a human, e.g., orally, topically, transdermally, parenterally, by inhalation spray, vaginally, rectally, or by intracranial injection. (The term parenteral as used herein includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and/or surgical implantation at a particular site is contemplated as well.) Generally, this will also entail preparing compositions that are essentially free of pyrogens, as well as other impurities that could be harmful to humans or animals. The term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art.

Therapeutic formulations of the compositions useful for practicing the invention such as polypeptides, polynucleotides, or antibodies may be prepared for storage by mixing the selected composition having the desired degree of purity with optional physiologically pharmaceutically acceptable carriers, excipients, or stabilizers (Remington's Pharmaceutical Sciences, 18th edition, A. R. Gennaro, ed., Mack Publishing Company (1990)) in the form of a lyophilized cake or an aqueous solution. Pharmaceutical compositions may be produced by admixing with one or more suitable carriers or adjuvants such as water, mineral oil, polyethylene glycol, starch, talcum, lactose, thickeners, stabilizers, suspending agents, etc. Such compositions may be in the form of solutions, suspensions, tablets, capsules, creams, salves, ointments, or other conventional forms.

Acceptable carriers, excipients or stabilizers are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

The composition to be used for in vivo administration should be sterile. This is readily accomplished by filtration through sterile filtration membranes, prior to or following lyophilization and reconstitution. Therapeutic compositions generally are placed into a container having a sterile access port, for example, an intravenous solution bag or vial having a stopper pierceable by a hypodermic injection needle. The route of administration of the composition is in accord with known methods, e.g. oral, injection or infusion by intravenous, intraperitoneal, intracerebral, intramuscular, intraocular, intraarterial, or intralesional routes, or by sustained release systems or implantation device. Where desired, the compositions may be administered continuously by infusion, bolus injection or by implantation device. The composition for parenteral administration ordinarily will be stored in lyophilized form or in solution.

The pharmaceutical forms suitable for injectable use include sterile aqueous solutions or dispersions and sterile powders for the extemporaneous preparation of sterile injectable solutions or dispersions. In all cases the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Suitable examples of sustained-release preparations include semipermeable polymer matrices in the form of shaped articles, e.g. films, or microcapsules. Sustained release matrices include polyesters, hydrogels, polylactides (U.S. Pat. No. 3,773,919, EP 58,481), copolymers of L-glutamic acid and gamma ethyl-L-glutamate (Sidman, et al., Biopolymers, 22: 547-556 (1983)), poly (2-hydroxyethyl-methacrylate) (Langer, et al., J. Biomed. Mater. Res., 15:167-277 (1981) and Langer, Chem. Tech., 12:98-105 (1982)), ethylene vinyl acetate (Langer, et al., supra) or poly-D(−)-3-hydroxybutyric acid (EP 133,988). Sustained-release compositions also may include liposomes, which can be prepared by any of several methods known in the art (e.g., DE 3,218,121; Epstein, et al., Proc. Natd. Acad. Sci. USA, 82:3688-3692 (1985); Hwang, et al., Proc. Natd. Acad. Sci. USA, 77:4030-4034 (1980); EP 52,322; EP 36,676; EP 88,046; EP 143,949).

An effective amount of the compositions to be employed therapeutically will depend, for example, upon therapeutic objectives, the route of administration, and the condition of the patient. A therapist can titer the dosage and modify the route of administration to obtain the optimal therapeutic effect. A typical daily dosage may range from about 1 μg/kg to up to 100 mg/kg or more, depending on the factors mentioned above. Typically, a clinician will administer the composition until a dosage is reached that achieves the desired effect. The progress of this therapy is easily monitored by conventional assays designed to evaluate the particular disease state being treated.

### B. Kits And Unit Doses

In related variations of the preceding embodiments, a binding construct may be packaged or formulated together with another binding construct or other therapeutic (e.g., an immunosuppressive agent), e.g., in a kit or package or unit dose, to permit co-administration, but these two components are not in admixture. In some embodiments, the two components to the kit/unit dose are packaged with instructions for administering the two compounds to a human subject for treatment of one of the disorders and diseases described herein.

### C. Polynucleotide-Based Therapies

The present invention also includes gene therapy materials and methods. Specifically, polypeptides and binding constructions of the invention can be produced at therapeutic levels in vivo by administration of a gene therapy contrast that enters cells and is expressed in vivo to produce the polypeptides or binding constructs. For example, in some embodiments, the vasculature of a cancer cell or cancer cells may be contacted with an expression construct capable of providing a therapeutic peptide or binding constructs of the present invention. Expression of the polypeptide or binding construct causes a therapeutic outcome, for example, inhibition of growth factors and receptors in the vasculature of a tumor, an inhibition of angiogenesis, an inhibition of lymphangiogenesis, an ablation, regression or other inhibition of tumor growth, an induction of apoptosis of the blood or lymphatic vasculature of the tumor or indeed the tumor cells themselves.

For these embodiments, an exemplary expression construct comprises a virus or engineered construct derived from a viral genome. Such vectors and constructs are considered aspect of the invention. The expression construct generally comprises a nucleic acid encoding the gene or binding construct, including any nucleic acid molecule described herein, to be expressed and also additional regulatory regions that will effect the expression of the gene in the cell to which it is administered. Such regulatory regions include for example promoters, enhancers, polyadenylation signals and the like.

DNA may be introduced into a cell using a variety of viral vectors. In such embodiments, expression constructs comprising viral vectors containing the genes of interest may be adenoviral (see, for example, U.S. Pat. Nos. 5,824,544; 5,707,618; 5,693,509; 5,670,488; 5,585,362, each incorporated herein by reference), retroviral (see, for example, U.S. Pat. Nos. 5,888,502; 5,830,725; 5,770,414; 5,686,278; 4,861,719, each incorporated herein by reference), adeno-associated viral (see, for example, U.S. Pat. Nos. 5,474,935; 5,139,941; 5,622,856; 5,658,776; 5,773,289; 5,789,390; 5,834,441; 5,863,541; 5,851,521; 5,252,479, each incorporated herein by reference), an adenoviral-adenoassociated viral hybrid (see, for example, U.S. Pat. No. 5,856,152 incorporated herein by reference) or a vaccinia viral or a herpesviral (see, for example, U.S. Pat. Nos. 5,879,934; 5,849,571; 5,830,727; 5,661,033; 5,328,688, each incorporated herein by reference) vector. Other vectors described herein may also be employed. Replication-deficient viral vectors are specifically contemplated.

In other embodiments, non-viral delivery is contemplated. These include calcium phosphate precipitation (Graham and Van Der Eb, Virology, 52:456-467 (1973); Chen and Okayama, Mol. Cell Biol., 7:2745-2752, (1987); Rippe, et al., Mol. Cell Biol., 10:689-695 (1990)), DEAE-dextran (Gopal, Mol. Cell Biol., 5:1188-1190 (1985)), electroporation (Tur-Kaspa, et al., Mol. Cell Biol., 6:716-718, (1986); Potter, et al., Proc. Nat. Acad. Sci. USA, 81:7161-7165, (1984)), direct microinjection (Harland and Weintraub, J. Cell Biol., 101:1094-1099 (1985)), DNA-loaded liposomes (Nicolau and Sene, Biochim. Biophys. Acta, 721:185-190 (1982); Fraley, et al., Proc. Natl. Acad. Sci. USA, 76:3348-3352 (1979); Felgner, Sci. Am., 276(6):102-6 (1997); Felgner, Hum. Gene Ther., 7(15):1791-3, (1996)), cell sonication (Fechheimer, et al., Proc. Natl. Acad. Sci. USA, 84:8463-8467 (1987)), gene bombardment using high velocity microprojectiles (Yang, et al., Proc. Natl. Acad. Sci. USA, 87:9568-9572 (1990)), and receptor-mediated transfection (Wu and Wu, J. Biol. Chem., 262:4429-4432 (1987); Wu and Wu, Biochemistry, 27:887-892 (1988); Wu and Wu, Adv. Drug Delivery Rev., 12:159-167 (1993)).

In a particular embodiment of the invention, the expression construct (or indeed the peptides discussed above) may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, “In Liver Diseases, Targeted Diagnosis And Therapy Using Specific Receptors And Ligands,” Wu, G., Wu, C., ed., New York: Marcel Dekker, pp. 87-104 (1991)). The addition of DNA to cationic liposomes causes a topological transition from liposomes to optically birefringent liquid-crystalline condensed globules (Radler, et al., Science, 275(5301):810-4, (1997)). These DNA-lipid complexes are potential non-viral vectors for use in gene therapy and delivery.

Liposome-mediated nucleic acid delivery and expression of foreign DNA in vitro has been very successful. Also contemplated in the present invention are various commercial approaches involving “lipofection” technology. In certain embodiments of the invention, the liposome may be complexed with a hemagglutinating virus (HVJ). This has been shown to facilitate fusion with the cell membrane and promote cell entry of liposome-encapsulated DNA (Kaneda, et al., Science, 243:375-378 (1989)). In other embodiments, the liposome may be complexed or employed in conjunction with nuclear nonhistone chromosomal proteins (HMG-1) (Kato, et al., J. Biol. Chem., 266:3361-3364 (1991)). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems that can be employed to deliver a nucleic acid encoding a therapeutic gene into cells include receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu (1993), supra).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu (1987), supra) and transferrin (Wagner, et al., Proc. Nat'l. Acad Sci. USA, 87(9):3410-3414 (1990)). Recently, a synthetic neoglycoprotein, which recognizes the same receptor as ASOR, has been used as a gene delivery vehicle (Ferkol, et al., FASEB. J., 7:1081-1091 (1993); Perales, et al., Proc. Natl. Acad. Sci., USA 91:4086-4090 (1994)) and epidermal growth factor (EGF) has also been used to deliver genes to squamous carcinoma cells (Myers, EPO 0273085).

In other embodiments, the delivery vehicle may comprise a ligand and a liposome. For example, Nicolau, et al., Methods Enzymol., 149:157-176 (1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also may be specifically delivered into a particular cell type by any number of receptor-ligand systems with or without liposomes.

In another embodiment of the invention, the expression construct may simply consist of naked recombinant DNA or plasmids. Transfer of the construct may be performed by any of the methods mentioned above that physically or chemically permeabilize the cell membrane. This is applicable particularly for transfer in vitro, however, it may be applied for in vivo use as well. Dubensky, et al., Proc. Nat. Acad. Sci. USA, 81:7529-7533 (1984) successfully injected polyomavirus DNA in the form of CaPO₄ precipitates into liver and spleen of adult and newborn mice demonstrating active viral replication and acute infection. Benvenisty and Neshif, Proc. Nat. Acad. Sci. USA, 83:9551-9555 (1986) also demonstrated that direct intraperitoneal injection of CaPO₄ precipitated plasmids results in expression of the transfected genes.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein, et al., Nature, 327:70-73 (1987)). Several devices for accelerating small particles have been developed. One such device relies on a high voltage discharge to generate an electrical current, which in turn provides the motive force (Yang, et al., Proc. Natl. Acad. Sci USA, 87:9568-9572 (1990)). The microprojectiles used have consisted of biologically inert substances such as tungsten or gold beads.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

Various routes are contemplated for various cell types. For practically any cell, tissue or organ type, systemic delivery is contemplated. In other embodiments, a variety of direct, local and regional approaches may be taken. For example, the cell, tissue or organ may be directly injected with the expression vector or protein.

Promoters for gene therapy for use in this invention include cytomegalovirus (CMV) promoter/enhancer, long terminal repeat (LTR) of retroviruses, keratin 14 promoter, and a myosin heavy chain promoter.

In a different embodiment, ex vivo gene therapy is contemplated. In an ex vivo embodiment, cells from the patient are removed and maintained outside the body for at least some period of time. During this period, a therapy is delivered, after which the cells are reintroduced into the patient; preferably, any tumor cells in the sample have been killed.

The techniques, procedures and methods outlined herein are applicable to any and all of the polypeptides and binding constructs of the present invention.

### D. Immunosuppressive And Other Combination Therapies

Any one of the binding constructs of the present invention when used in a method of treating or preventing a disease, e.g, a graft rejection or arteriosclerosis, may be employed alone, or in combination with other agents. In some embodiments, more than one binding construct may be administered. In some embodiments, a binding construct may be administered together with an immuno suppressive protein, antibody, nucleic acid, or chemotherapeutic agent.

Preferably, when used in combination with the endothelial growth factor inhibitor binding constructs of the present invention, the results obtained are synergistic. That is to say, the effectiveness of the combination therapy of a binding construct and the immunosuppressive agent is synergistic, i.e., the effectiveness is greater than the effectiveness expected from the additive individual effects of each. Therefore, the dosage of the immunosuppressive compound can be reduced and thus, the risk of the toxicity problems and other side effects is concomitantly reduced.

Any immunosuppressant therapy that has some efficacy at reducing transplant rejection alone can be used in combination with the inhibitors of the invention, and such combinations are specifically contemplated as combination therapies of the invention.

Corticosteroids are generally considered to be a first-line therapy for acute allograft rejection. Exemplary corticosteroids include prednisone and prednisolone, which are commonly used for prophylaxis against rejection, and methylprednisolone, which is often used for incidences of acute rejection. Dosage of corticosteroids is well developed by those in the field of transplant medicine, and varies depending on whether prescribed as initiation or maintenance therapy, and depending on patient tolerance. It is contemplated that reduced doses of corticosteroids may be used when combined with the inhibitors of the invention. The lower dosing is expected to help reduce known adverse effects of corticosteroids, which include hyperglycemia, diabetes mellitus, edema, hypertension, hyperlipidemia, hypokalemia, hirsutism, GI bleeding, arthralgia, osteoporosis, and psychosis.

A second class of immunosuppressants used with transplant patients, and contemplated for combination of therapy of the invention, is calcineurin inhibitors. Representative members of the class include cyclosporine and tacrolimus. These molecules act as immunosuppressive agents by binding to immunophilin molecules to inhibit calcineurin, and thereby inhibit T cell activation and proliferation. The patient's blood/serum and renal and liver functions are monitored to maintain an effective dose while minimizing toxicity. Combination of cyclosporine with other immunosuppressants is known to cause side-effects and may require dose modulation. The main side effects of CNIs are nephrotoxicity and neurotoxicity.

The mTOR inhibitors (mammalian target of rapamycin) represent a third class of immunosuppressants. The mTOR inhibitors, which include sirolimus and everolimus, inhibit T cell proliferation. Sirolimus has been used extensively in renal transplantation, and can be taken orally. Side effects associated with sirolimus therapy include dyslipidemia, hypertension, thrombocytopenia, anemia, peripheral edema, and tremor.

Antiproliferative agents represent a fourth class of immunosuppressants, and include azathioprine. A common starting dose for adults is 50 mg per day. After 4 to 8 weeks, the dose may be increased. Most adults require 50-150 mg per day. A dose-dependent bone marrow suppression may occur in over half of patients treated with azathioprine, and hepatotoxicity has been reported. Mycophenolic acid is another antiproliferative, which targets inosine monophosphate dehydrogenase (IMPDH), and causes a relatively selective suppression of lymphocyte proliferation. Compared to many of the other immunosuppressants, MPA lacks significant nephrotoxicity. The capsules come in 250 mg and 500 mg and the usual dose for adults is 1-1.5 g (2-6 capsules) twice a day. The dose for children is 15 mg/kg/day.

A fifth class of immunosuppressants used in transplant patients is anti-lymphocyte-depleting antibodies: Induction therapy prior to transplantation and during the first weeks post-transplantation with an antilymphocyte-depleting antibody or an IL-2 receptor (IL-2R) antagonist can provide effective protection against rejection. Polyclonal antilymphocyte-depleting antibodies, ATGAM and Thymoglobulin, sometimes are used after organ transplantation to reduce the risk of acute rejection. These antibodies are directed against T and B lymphocytes, and cause T cell lysis and blockade of B cell activation.

Muromonab C3 is a monoclonal antibody that is sometimes used in the treatment of acute rejection in transplant recipients. It binds to CD3 expressed on T cells and interferes with T cell antigen recognition.

IL-2R antagonists represent yet another class of immunosuppressants. Exemplary members of this class include basiliximab (Simulect) and daclizumab (Zenopax). The act by binding to the CD25 subunit of the IL-2R, to block T cell activation. They are used for prophylaxis, rather than treatment, of acute rejection because during acute rejection, T cells may become activated without the involvement of the CD25 subunit on the IL-2 receptor.

Mercaptopurine (6-MP) belongs to a group of drugs known as antimetabolites. It is used to treat many types of autoimmune diseases. It may interfere with the normal menstrual cycle in women and may stop sperm production in men. The usual adult dose is 2.5 mg/kg/day (100-200 mg). The pediatric dose is 50 mg per day. A maintenance dosage after remission is 1.5-2.5 mg/kg/day.

The foregoing list is not intended to be limiting. Inhibitors of the invention can be co-administered with any immunosuppressive agent to benefit from their complementary effects.

Other exemplary therapies to combine with therapies that target endothelial growth factors and growth factor receptors include those described in International Application No. PCT/EP2004/012406 (WO 2005/049021), incorporated herein by reference in its entirety, relating to a combination of an inhibitor of a mammalian Target of Rapamycin (mTOR), such as rapamycin; and an inhibitor of a Platelet-Derived Growth Factor Receptor (PDGF-R), such as imatinib mesylate.

mTOR inhibitors include, but are not limited to the following drugs: Brand Name or Product #Generic Name Patent (U.S. Unless Specified) or

| Brand Name | | Patent (U.S. Unless Specified) |
|---|---|---|
| or Product # | Generic Name | or Reference |
| Rapamune ® | rapamycin (sirolimus) | 3,929,992; 5,288,711; 5,516,781 |
| RAD001 | everolimus; 40-O-(2- | EP663916; U.S. Pat. Appl. |
| (Certican ®) | hydroxyethyl)-rapamycin | 20030170287 |
| CCI-779 | Rapamycin 42-ester with 3- | 6,617,333; 5,362,718; 6,277,983 |
| | hydroxy-2-(hydroxymethyl)-2- | |
| | methylpropionic acid | |
| | Tumstatin and related | U.S. Pat. Appl. 20030144481 |
| | polypeptides | |
| ABT578 | | U.S. Pat. Appl. 20030073737 |
| “rapalogs,” | | U.S. Pat. Appl. 20030073737; |
| e.g., AP23573, | | WO01/02441; WO01/14387 |
| AP22594 | | |
| AP23841 | | ARIAD Pharmaceuticals |
| TAFA93 | | Isotechnika |

In addition to rapamycin and those derivatives of rapamycin listed in the above table those discussed in U.S. Pat. Appl. No. 20030170287 may also be used. See also WO 94/09010, and WO 96/41807. Rapamycin derivatives may also include without limitation “rapalogs,” e.g., as disclosed in WO 98/02441 and WO01/14387; deuterated rapamycin analogs, e.g., as disclosed in U.S. Pat. No. 6,503,921. Derivatives of other mTOR inhibitors are also contemplated.

Exemplary Platelet-Derived Growth Factor Receptor (PDGF-R) inhibitors include the following without limitation:

| Brand Name | | Patent (U.S. Unless Specified) |
|---|---|---|
| or Product # | Generic Name | or Reference |
| Gleevec | imatinib mesylate | 5,521,184; Druker, Nature Medicine, |
| | (CGP57148B; STI-571) | 2: 561 (1996) |
| AG 1295 | | Kovalenko, Cancer Res 54: 6106 |
| | | (1994) |
| AG 1296 | 6,7-demethoxy-2- | Kovalenko, Cancer Res 54: 6106 |
| | phenylquinoxaline | (1994) |
| AG 1478 | 4(3-chlorophenyamino)-6,7,- | Lipson, K., et al., J. Pharmacology and |
| | dimethoxyquinazoline | Experimental Therapeutics, 285: 844-852 |
| | | (1998) |
| Trapidil | triazolopyrimidine | Lotinum et al., Endocrinology, 144: 2000-2007 |
| | | (2003) |
| | 2-phenylaminopyrimidine | Buchdunger et al., Cancer Res., |
| | class compounds | 56: 100-1044 (1996) |
| 3744W | | Spacey et al., Bioch. Pharm. |
| | | 55(3): 261-71, 1998 Feb. 1 |
| Tyrphostin | | Kovalenko et al, Cancer Res. |
| AG1296 | | 1994 Dec. 1; 54 (23) |
| CGP 79787D | | WO00/09098 |
| CGP53′716 | | Buchdunger et al., PNAS 1995 |
| | | 92(7): 2558-62 |
| CGP57′148 | | Buchdunger et al., Cancer Res. 1996 |
| | | 56(1): 100-4 |
| CT52923 | | U.S. Pat. Appl. No. 20030170287; |
| | | Lokker et al., Cancer Res., 62: 3729-35 |
| | | (2002). |
| RP-1776 | | U.S. Pat. Appl. No. 20030170287; |
| | | Toki et al., J Antibiot (Tokyo). |
| | | 54: 405-14 (2001) |
| GFB-111 | | U.S. Pat. Appl. No. 20030170287; |
| | | Blaskovich et al., Nat Biotechnol. |
| | | 18: 1065-70 (2000). |
| | a pyrrolo[3,4-c]-beta- | U.S. Pat. Appl. No. 20030170287; |
| | carboline-dione | Teller et al., Eur J Med Chem., 35: 413-27 |
| | | (2000) |
| SU11248 (SU01248) | | Sugen Pharmaceutical; Abrams et al., |
| | | Mol Cancer Ther. 2: 1011-21 (2003); |
| | | Mendel et al. Clin Cancer Res. 9: 327- |
| | | 37 (2003). |
| PKC787 | | Novartis |
| PTK787 | | Novartis; U.S. Pat. Appl. No. |
| | | 20030087934; Wood et al., Cancer |
| | | Res. 60: 2178-89 (2000). |
| DMBI | | Organon; Zaman, Biochem. |
| | | Pharmacol. 57: 57 1999 |
| SU101 (LFM, HWA486) | | Sugen; Shawner, Clin. Cancer. Res., |
| | | 3: 1167 1997 |
| SU0020 (A771726) | | Sugen; Zhang et al., J. Pharm. Biomed. |
| | | Anal. 28: 701-9 (2002). |
| Comp. 54 | | Parke-Davis; Boschelli, J. Med. Chem. |
| | | 41: 4365 (1998) |

The above list of PDGF-R inhibitors is not meant to be limiting. Any PDGF-R inhibitor may be employed, including without limitation PDGF-R inhibitors described in U.S. Pat. Nos. 5,932,580, 6,331,555, and 6,358,954; WO 99/28304; WO 00/09098; WO 01/64200. Other inhibitors that may be used include 3-Substituted Indolin-2-ones (e.g., SU5416, SU6668), and derivatives thereof (Sun et al., J. Med. Chem., 41:2588-2603; Sun et al., J. Med. Chem. 43:2655-2663 (2000)); 2-Amino-8H-pyrido[2,3-d]pyrimidines (Boschelli et al., J. Med. Chem. 41:4365-4377 (1998)).

In some embodiments, the PDGF-R inhibitor is a compound described in U.S. Pat. No. 5,521,184, incorporated herein by reference.

In addition to or in substitution for PDGF-R inhibitors, inhibitors of other tyrosine kinases (receptors and other types as well) may also be used in accordance with this invention. Some of these inhibitors may inhibit multiple kinases including, but not limited to, PDGF-Rs. Appropriate TK inhibitors are also taught in WO 99/03854; WO 01/64200; U.S. Pat. No. 5,521,184; WO 00/42042; WO 00/09098; EP 0 564 409 B1; U.S. Pat. No. 5,521,184; WO 97/32604; U.S. Pat. No. 6,610,688; US Patent Appl. Pub. No. 20030194749; Livitzki, A., et al., “Protein Tyrosine Kinase Inhibitors as Novel Therapeutic Agents, “Pharmacol. Ther. 82:231-29 (1999). Other classes of compounds may also be employed. For example and without limitation, Leflunomide (U.S. Pat. No. 4,284,786) and/or derivative FK778 may be used. (See, e.g., Savikko Transplantation 2003:76:455 and editorial Williams ibid p 471.)

### E. Suitable Transplant Recipients

The present invention is applicable to all cell, tissue, organ fragment, organ, and multi-organ transplant procedures, to inhibit or delay rejection reactions or other undesired side-effects, including arteriosclerosis, that are associated with transplants. Thus, the invention is applicable to any mammal that receives any cell, tissue, organ fragment, organ, or multi-organ transplant.

Exemplary transplants include autografts (transplant or transfer of tissue from an organism to itself, e.g., where donor and recipient are the same organism); isografts (transplants from a donor organism to a genetically identical recipient (e.g., an identical twin or a clone); allografts (transplants from a donor organism to a genetically non-identical organism of the same species); and xenografts (transplants of organs or tissue from one species to another). All of these types of transplants are theoretically possible in humans, although xenografts have thus far been fairly limited (e.g., heart valves), and isograft donors are quite rare. Thus, in the context of transplantation of vital organs or tissues to replace diseased ones, allograft transplants represent the most common class for human therapy. The likelihood of rejection or graft arteriosclerosis increases as the differences between donor and recipient increase.

Exemplary organs that have been transplanted in humans, and for which the methods of the invention are especially applicable, include thoracic organs (e.g., heart, lung); abdominal organs (e.g., liver, kidney, pancreas, small intestine). The invention also is applicable for various tissue and cell transplants, including but not limited to pancreatic islet cells, bone marrow cells, cardiac myocytes, blood vessels or vessel fragments, heart valves, bones, and skin. The invention also is applicable to the emergent fields of transplantation of embryonic stem cells and various pluripotent or multipotent progenitor or precursor cells that have the potential to differentiate into one or more cell types (e.g., hematopoietic progenitor/stem cells, neural progenitor/stem cells, endothelial progenitor cells, and muscle progenitor cells).

### F. Transplant Rejection and Monitoring of Transplanted Tissue

After surgery, the transplanted cell, tissue or organ is carefully monitored for any signs that the recipient will reject the new cell, tissue or organ. There are three main types of transplant rejections: (1) hyperacute, (2) acute, and (3) chronic transplant rejections.

Hyperacute rejection is a complement-mediated response in recipients with pre-existing antibodies to the donor (for example, ABO blood type antibodies). Hyperacute rejection occurs within minutes and the transplant must be immediately removed to prevent a severe systemic inflammatory response. Rapid coagulation of the blood occurs. This is a particular risk in kidney transplants, and so a prospective cytotoxic crossmatch is performed prior to kidney transplantation to ensure that antibodies to the donor are not present. For other organs, hyperacute rejection is prevented by transplanting only ABO-compatible grafts. Hyperacute rejection is the likely outcome of xenotransplanted organs.

Acute rejection is generally acknowledged to be mediated by T cell responses to proteins from the donor organ, which differ from those found in the recipient. Unlike antibody-mediated hyperacute rejection, development of T cell responses first occurs several days after a transplant if the patient is not taking immunosuppressant drugs. Since the development of powerful immunosuppressive drugs, such as those discussed above, the incidence of acute rejection has been greatly decreased. However, organ transplant recipients can develop acute rejection episodes months to years after transplantation. Acute rejection episodes can destroy the transplant if it is not recognized and treated appropriately. A single episode is not a cause for grave concern if recognized and treated promptly, and rarely leads to organ failure, but recurrent episodes are associated with chronic rejection of grafts.

The bulk of the immune system response is to the Major Histocompatibility Complex (MHC) proteins. MHC proteins are involved in the presentation of foreign antigens to T cells, and receptors on the surface of the T cell (TCR) are uniquely suited to recognition of proteins of this type. MHC are highly variable between individuals, and therefore the T cells from the donor recognize the foreign MHC with a very high frequency, leading to powerful immune responses that cause rejection of transplanted tissue. Identical twins and cloned tissue are MHC matched, and are therefore not subject to T cell mediated rejection.

The term “chronic rejection” is used when the process is shown to be due to a chronic alloreactive immune response. It can be caused by a member of the Minor Histocompatibility Complex such as the H-Y gene of the male Y chromosome, and usually leads to the need for a new organ after a decade or so.

Cardiac Allograft vasculopathy (CAV), also known as chronic cardiac rejection or transplant coronary artery disease, is the main factor limiting the long term success of heart transplants, and most likely involves both immunological and non-immunological factors (Al-Khaldi et al., Annu. Rev. Med., 57:455-471, 2006, the disclosure of which is incorporated herein by reference). Identified risk factors include older donor and recipient age, ischemia-reperfusion injury, human leukocyte antigen (HLA) mismatch, hypertension, hyperlipidemia, insulin resistance, cytomegalovirus infection, and recurrent rejection (Taylor et al., J. Heart Lung Transplant., 23:796-803, 2004; Valantine et al., Circ., 103:2144-2152, 2001; Costanzo-Nordin et al., J. Heart. Lung Transplant., 11:S90-103, 1992; Grattan et al., JAMA, 261:3561-3566, 1989; Kobashigawa et al., J. Heart Lung. Transplant., 14:S221-S226, 1995; and Valantine, H., J. Heart Lung. Transplant., 23:S187-S193, 2004). These risk factors may lead directly or indirectly to endothelial injury with subsequent intimal hyperplasia and vascular smooth muscle proliferation.

Monitoring of heart transplant recipients for the development of allograft rejection includes non-invasive methods such as intramyocardial electrocardiogram (Hetzer et al., Ann Thorac Surg 66:1343, 1998) and echocardiography, radioisotope techniques, magnetic resonance imaging and immunological methods (Kemkes et al., J Heart Lung Transplant 11: S221-31, 1992). The immunological methods include the measurement of serum cytokine levels, particularly IL6 and IL8 (Kimball et al., Transplantation 61: 909-15, 1996), monitoring recipient serum for donor HLA antigens and anti-HLA antibodies (Reed et al., Transplantation 61: 566-72, 1996), and measuring reactivity of allo-reactive helper T cells (DeBruyne, Transplantation 56: 722-7, 1993), or cytotoxic T cells in the blood of the recipient (Reader et al. Transplantation 50: 29-33, 1990; Loonen et al., Transplant Int 7:596-598, 1994). Invasive methods include an endomyocardial biopsy, which detects an ongoing immune rejection process that may have already damaged the heart before immunosuppressive intervention has been initiated. Non-invasive methods are associated with the detection of ongoing damage in the heart muscle and thus, may come too late for the preferred goal in improving the care of the transplant recipient: early prevention of the developing rejection episode.

The monitoring methods used in other organ allograft recipients are also directed to detection of damage to the transplanted organ. With respect to kidney allograft recipients, noninvasive methods include the functional indicators of impaired renal activity, such as (a) decreased urine volume, (b) decreased clearance of creatinine and (c) elevated blood urea nitrogen. Monitoring includes detection of lymphocytes in the urine (Salaman, Immunol Lett 29: 139-12, 1991), secretion of neopterin (a pteridine from stimulated macrophages) and interferon-gamma (a cytokine released by activated T cells) (Khoss et al. Child Nephrol Urol 9:46-49, 1988; Grebe et al., Cuff Drug Metabol 3:189-202, 2002). The sensitivity of detection of inflammatory products in the urine was further improved by measuring the presence of mRNA for perforin and granzyme B (proteins released from T cells that damage target cells), using the reverse transcriptase-polymerase chain reaction (RT-PCR) for amplification and detection of these molecules (Li et al., N Engl J Med 344: 947-954, 2001).

### V. NON-EXCLUSIVE EXAMPLES OF THE INVENTION

The invention may be more readily understood by reference to the following examples, which are given to illustrate the invention and not in any way to limit its scope. The first several examples describe making and testing inhibitor compounds useful to practicing methods of the invention. The second group of examples describe evidence that the antigens are suitable targets for therapy and/or evidence that such therapy is efficacious. These examples primarily make reference to binding constructs that bind particular growth factors of the VEGF subfamily, but they may also be adapted for use of binding constructs that bind other VEGF subfamily members, as well as for binding constructs that bind PDGF subfamily members. Similarly, binding constructs comprising other VEFGR receptor fragments, PDGFR receptor fragments, and neuropilin receptor fragments may also be employed in variations of these examples.

### Example 1

VEGFR-2 and VEGFR-3 Fragments that Bind

### VEGF-A or VEGF-C

To determine the portion of a receptor's extracellular domain (ECD) that was sufficient for ligand binding, fragments of the ECDs of VEGFR-2 (R-2) and VEGFR-3 (R-3) were used to make various soluble constructs. The constructs included Fc domain human IgG fragments fused to the C-terminus of the receptor fragments. As indicated in Tables 3 and 4, some constructs were made using a heterologous (N-terminal) signal peptide derived from CD33.

### Construction of Fragments and Plasmids

### R-2 Constructs

To construct the VEGFR-2/IgG expression plasmid, the construct, R-2 A, comprising the first three Ig-domains (D1-3) of VEGFR-2 was amplified by PCR using primers 5′-GCGGATCCTTGCCTAGTGTTTCTCTTGATC-3′ (SEQ ID NO: 72), and 5′-CCAGTCACCTGCTCCGGATCTTCATGGACCCTGACAAATG-3′ (SEQ ID NO: 73), and cloned into the Signal pIgplus vector (Novagen, Madison, WI). The resulting plasmid was digested with BamHI and KpnI, treated with T4 polymerase and back-ligated. To assemble other VEGFR-2/IgG constructs, PCRs were performed using the D1-3 construct as the template, T7 forward primer and the following reverse primers:

(SEQ ID NO: 59)
5′-GCTGGATCTTGAACATAGACATAAATG-3′ (R-2 F),,
(SEQ ID NO: 60)
5′-CTAGGATCCCCTACAACGACAACTATG-3′ (R-2 B),,
(SEQ ID NO: 61)
5′-CTAGGATCCACATCATAAATCCTATAC-3′(R-2 C),,
(SEQ ID NO: 62)
5′-GCATGGTCTCGGATCATGAGAAGACGGACTCAGAAC-3′
(R-2 D),,
(SEQ ID NO: 63)
5′-CTAGGATCCTTTTCTCCAACAGATAG-3′ (R-2 E);
forward primer
(SEQ ID NO: 64)
5′-AGCGCTAGCGTTCAAGATTACAGATCTCC-3′,
and
the following reverse primers:
(SEQ ID NO: 65)
5′-ATGTGTGAGGTTTTGCACAAG-3′(R-2 G),,
(SEQ ID NO: 66)
5′-CTAGGATCCCCTACAACGACAACTATG-3′(R-2 H),,
(SEQ ID NO: 67)
5′-CTAGGATCCACATCATAAATCCTATAC-3′(R-2 I),,
(SEQ ID NO: 68)
5′-GCATGGTCTCGGATCATGAGAAGACGGACTCAGAAC-3′
(R-2 J),,
(SEQ ID NO: 69)
5′-CTAGGATCCTTTTCTCCAACAGATAG-3′(R-2 K),,
forward primer
(SEQ ID NO: 70)
5′-AGCGCTAGCTATAGGATTTATGATGTG-3′,
and
reverse primer
(SEQ ID NO: 71)
5′-ATGTGTGAGGTTTTGCACAAG-3′(R-2 L),.

The PCR products were digested with NheI and BstYI (R-2 F and L constructs), NheI and BamHI (R-2 E, and H-K constructs), BamHI (R-2 linker B and C constructs), BamHI and BsaI (R-2 D construct), or NheI and BsmBI (R-2 G construct), and cloned into the Signal pIgplus vector. In order to repair frame-shifts in constructs containing nucleotide sequence coding for domain 1 of VEGFR-2, the vectors were cut with restriction enzyme NotI, blunted with Klenow enzyme, cut with EcoRV and back-ligated.

### R-3 Constructs

A series of R-3 constructs with C-termini between Ig domains 2 and 3 of VEGFR-3 (R-3 C through F constructs) was created by PCR using the expression plasmid comprising the R-3 D1-3 transcript (e.g., the R-3 G construct, SEQ ID NO: 43) as template, T7 as forward primer and the following reverse primers:

(SEQ ID NO: 74)
5′-TCAGGATCCGCGAGCTCGTTGCCTG-3′,
(SEQ ID NO: 75)
5′-TACAGGATCCCCTGTGATGTGCACCAG-3′,
(SEQ ID NO: 76)
5′-TCAGGATCCGCGTGCACCAGGAAGG-3′,
and
(SEQ ID NO: 77)
5′-TCAGGATCCGCGAAGGGGTTGGAAAG-3′.

The Ig homology domain 1 was deleted from the D1-3 expression plasmid (R-3 G construct) by site-directed mutagenesis using primers

(SEQ ID NO: 78)
5′CCTTGAACATCACGGAGGAGTCACACGTCAGAGACTTTGAGCAGCCAT
TCATCAACAAGC-3′
and
(SEQ ID NO: 79)
5′AGCTGCTGGTAGGGGAGAAGGATCCTGAACTGCACCGTGTGG-3′,

and excision of the BamH I fragment from the resulting plasmid. That procedure combined with the described truncation primers, for R-3 C through F constructs, allows for the production of the R-3 constructs (e.g., C, D, E, F, J, K, L, and M). The plasmid coding for domains 2 and 3 of VEGFR-3 (R-3 I) was made by transfer of the Sph I fragment from the original expression R-3 D1-3 plasmid into the plasmid encoding only domain 2 of VEGFR-3 (R-3 J). The sequence derived from a particular receptor is listed in Table 2. Expression was performed using standard calcium phosphate-mediated transfection into 293T cells.

The binding assays utilized minimal VEGF-A (SEQ ID NOS: 106 and 107) and VEGF-C(SEQ ID NOS: 108 and 109) fragments with 109 residues each (called VEGF-A 109 and VEGF-C 109). These constructs are not naturally occurring, but are effective for binding assays. Other growth factor constructs, either natural or artificial, may also be used for performing these assays.

Either Tritiated VEGF-A 109 or VEGF-C 109 was used in a given binding experiment. Ligand in solution was precipitated by mixing 175 μl of ligand solution with 100 μl binding mix at 4° C. overnight, with agitation. The ligand solution may be the supernatant of metabolically labeled 293T cells. The binding mixes used for the receptor binding analysis were as follows: for VEGFR-1 binding assays, the binding mix was phosphate buffered saline (PBS) containing 1.5% BSA, 0.06% Tween 20, 3 μg/ml heparin and 400 ng/ml VEGFR-1-Fc fusion protein (100 μl of this binding mix was added to 200 μl of ligand solution). For VEGFR-2 binding assays, the binding mix was 82% conditioned cell supernatant from 293T cells transiently expressing VEGFR-2-Fc fusion protein in mixture with 18% of a PBS solution that contained 5% BSA, 0.2% Tween 20, and 10 μg/ml heparin (250 μl of binding mix was added to 200 μl of ligand solution). For VEGFR-3 binding assays, the binding mix was 82% conditioned cell supernatant from 293T cells transiently expressing VEGFR-3-Fc fusion protein, 18% of PBS containing 5% BSA, 0.2% Tween 20, and 10 μg/ml heparin (250 μl of binding mix was added to 200 μl of ligand solution). To collect precipitated ligand, 50 μl of a 30% protein A sepharose (PAS, Pharmacia) slurry in PBS was added and incubated under agitation for at least 1.5 hr at 4° C. Standard buffer was added to each immunoprecipitation sample and boiled for 5 minutes at 95° C. during which the immunopreciptated proteins become dissociated from the protein A sepharose. After centrifugation, 10 μl of each sample was analyzed on 15% SDS-PAGE under reducing conditions. The gels were dried and exposed for either 12 hours on phosphorimager plates or 4 weeks on X-ray film.

Tables 3 and 4 identify constructs by name, a DNA and deduced amino acid sequence from the sequence listing, the portion of VEGFR-2 (SEQ ID NO: 4) or VEGFR-3 (SEQ ID NO: 6) amino acid sequence that was included in the constructs, whether the constructs expressed, and, if tested, whether constructs bound ligand. The table data is compiled from analysis of PAGE gels. The asterisk adjacent to the “B*” indicates a “spill-over” from the adjacent lane, as the origin of the bands seen in the “B” lane. A failure to express under the particular experimental conditions used in this instance should not be interpreted as a failure to bind. The experiments can be repeated using different receptor fragments, binding constructs, ligands, or combinations thereof.

TABLE 3
VEGFR-2 CONSTRUCTS

| | | SEQ ID | | Binds | Binds |
|---|---|---|---|---|---|
| Fc Fusion Constructs | SEQ ID NOS: | NO: 4 | Expression | VEGF-A | VEGF-C |
| R-2 A | SEQ ID NOS: | 24-326 | Yes | Yes | Yes |
| with CD33 | 7 and 8 | | | | |
| Signal Peptide | | | | | |
| R-2 B | SEQ ID NOS: | 24-220 | Yes | No | No |
| with CD33 | 9 and 10 | | | | |
| Signal Peptide | | | | | |
| R-2 C | SEQ ID NOS: | 24-226 | Yes | No | No |
| with CD33 | 11 and 12 | | | | |
| Signal Peptide | | | | | |
| R-2 D | SEQ ID NOS: | 24-232 | Yes | No | No |
| with CD33 | 13 and 14 | | | | |
| Signal Peptide | | | | | |
| R-2 E | SEQ ID NOS: | 24-241 | Yes | No | No |
| with CD33 | 15 and 16 | | | | |
| Signal Peptide | | | | | |
| R-2 F | SEQ ID NOS: | 24-122 | Yes | No | No |
| with CD33 | 17 and 18 | | | | |
| Signal Peptide | | | | | |
| R-2 G | SEQ ID NOS: | 118-326 | Yes | Yes | Yes |
| with CD33 | 19 and 20 | | | | |
| Signal Peptide | | | | | |
| R-2 H | SEQ ID NOS: | 118-220 | Yes | No | Yes |
| with CD33 | 21 and 22 | | | | |
| Signal Peptide | | | | | |
| R-2 I | SEQ ID NOS: | 118-226 | Yes | No | Weak |
| with CD33 | 23 | | | | |
| Signal Peptide | and 24 | | | | |
| R-2 J | SEQ ID NOS: | 118-232 | Yes | No | Very Weak |
| with CD33 | 25 and 26 | | | | |
| Signal Peptide | | | | | |
| R-2 K | SEQ ID NOS: | 118-241 | Yes | No | No |
| with CD33 | 27 and 28 | | | | |
| Signal Peptide | | | | | |
| R-2 L | SEQ ID NOS: | 220-326 | Yes | No | No |
| with CD33 | 29 and 30 | | | | |
| Signal Peptide | | | | | |

TABLE 4
VEGFR-3 CONSTRUCTS

| Fc Fusion | Sequence ID | SEQ ID | | |
|---|---|---|---|---|
| Constructs | Nos. | NO: 6 | Expression | Binds VEGF-C |
| R-3 A with CD33 | SEQ ID NOS: | 138-329 | Yes | — |
| Signal Peptide | 31 and 32 | | | |
| R-3 B with CD33 | SEQ ID NOS: | 138-226 | Yes | Yes |
| Signal Peptide | 33 and 34 | | | |
| R-3 C | SEQ ID NOS: | 1-229 | Yes | Yes |
| | 35 and 36 | | | |
| R-3 D | SEQ ID NOS: | 1-226 | Yes | Yes |
| | 37 and 38 | | | |
| R-3 E | SEQ ID NOS: | 1-223 | No | — |
| | 39 and 40 | | | |
| R-3 F | SEQ ID NOS: | 1-220 | No | — |
| | 41 and 42 | | | |
| R-3 G | SEQ ID NOS: | 1-329 | Yes | Yes |
| | 43 and 44 | | | |
| R-3 H | SEQ ID NOS: | 1-134 | Yes | No |
| | 45 and 46 | | | |
| R-3 I | SEQ ID NOS: | 1-39, 132-329 | Yes | No |
| | 47 and 48 | | | |
| R-3 J | SEQ ID NOS: | 1-39, 132-247 | Yes | No |
| | 49 and 50 | | | |
| R-3 K | SEQ ID NOS: | 1-39, 132-229 | Yes | No |
| | 51 and 52 | | | |
| R-3 L | SEQ ID NOS: | 1-39, 132-226 | Yes | — |
| | 53 and 54 | | | |
| R-3 M | SEQ ID NOS: | 1-39, 132-223 | No | — |
| | 55 and 56 | | | |
| R-3 N | SEQ ID NOS: | 1-40, 226-329 | — | — |
| | 57 and 58 | | | |

The results of these assays demonstrate that novel receptor fragments are capable of binding ligands that the receptor as a whole may bind. In addition to providing a clearer picture as to what regions of the ECD are necessary for ligand binding, the binding data identifies receptor fragments useful as therapeutics.

The present data show that the R-2 H fragment of R-2 of approximately 100 residues and spanning D2 of R-2 is sufficient for VEGF-C binding. For R-3, a larger fragment is required for VEGF-C binding, e.g., the R-3 D construct in table 4, which spans D1-2 of R-3.

Three-dimensional modeling based on the structure of VEGFR-1 complexed with VEGF-A was used to predict that a groove in VEGF-C might accommodate the region between Ig-like domains 2 and 3 of VEGFR-3 (Flt4). WO 01/62942. The present data shows for the first time that sequence intermediate between the second and third Ig domains of R-3 is important for ligand binding.

For R-1 and R-2, the first Ig-domain has been described as inhibitory for VEGF-A binding. Lu, et al., J. Biol. Chem., 275(19): 14321-14330 (2000); Shinkai, A. et al., J. Biol. Chem., 273(47):31283-88 (1998). For VEGF-C binding, the present data show that the inhibitory role of the first Ig-domain appears to apply to R-2 fragments, but not R-3 fragments.

The data also provides novel information regarding R-2 fragments and VEGF-A binding. Conflicting reports exist for constructs comprising the second and third Ig-domains of R-2 and VEGF-A binding. Fuh, et al., J. Biol. Chem., 273(18): 11197-11204 (1998); Niwa, et al., U.S. Pat. No. 6,348,333; Shinkai, A. et al., J. Biol. Chem., 273(47):31283-88 (1998). Fuh reported that only domains 2 and 3 were needed. Niwa taught that only 1 and 2 were needed. Shinkai stressed the importance of domain 4 of R-2. The issue is further confused because different reports have defined the boundaries of the Ig-domains in different ways, i.e., different start and stop points, a practice that has been recognized as potentially affecting whether fragments bind ligands, and with what degree of affinity. Shinkai, A. et al., J. Biol. Chem., 273(47):31283-88 (1998).

### Example 2

Ligand Binding Assays Involving Binding Constructs with More than One Binding Element

The assays as performed in Example 1 are repeated, substituting a binding construct with multiple binding units. For example, one employs a binding construct comprising a binding unit that binds VEGF-A and a binding unit that binds VEGF-C. One looks for the ability of such a binding construct to bind both VEGF-A and VEGF-C. This information may be obtained by using different radio- or other labels, e.g., fluorescent labels for fluorescence resonance energy transfer (FRET), on each type of ligand or use of labels on the binding construct and or ligands, to determine whether a given binding construct molecules are binding a molecule of VEGF-A and VEGF-C. Constructs that are shown to bind more than one growth factor ligand, as well as those described in Example 1 and elsewhere herein, have an indication for anti-neoplastic therapies where multiple growth factors contribute to neoplastic cell growth.

### Example 3

Chimeric Vegfr Binding Constructs which Bind Multiple Ligands

As stated above, constructs that bind more than one growth factor ligand have an indication as anti-neoplastic therapies where multiple growth factors contribute to neoplastic cell growth. In order to determine the efficacy of a binding construct designed to bind more than one growth factor, two chimeric binding constructs were generated and their ability of each to bind to two growth factors was measured.

The binding constructs were designed as immunoblobulin fusion proteins as described above. To construct chimeric VEGF receptor/hIgG1Fc fusion proteins, the pIgPlus vector was used to build a construct comprising the first immunoglobulin-like domain of VEGFR-3 and the second and third Ig-like domains of VEGFR-2. The construct is designated R-3D1-R2D2+3/hIgG1Fc. To clone the R-3D1-R2D2+3/hIgG1Fc construct, PCR was performed with CMV forward primer (18782, 5′ TACTTGGCAGTACATCTACGTATTAGTCATCGC-3′) (SEQ ID NO: 122) and reverse primer v360 (5′-CGGAGATCTGTAGTCTTGCACGTACACGTAGGAGCTGGC-3′) (SEQ ID NO: 123) using plgPlus-hVEGFR-3D1-3-IgG1Fc as a template. The PCR-product was cut with SnaBI and BglII. The 718 bp D1-R2D2+3/hIgG1Fc insert was ligated into the SnaBI- and partially BglII-cut vector plgPlus-hVEGFR-2D1-3-IgG1Fc described above. The presence and sequence of the correct insert was confirmed by sequencing a representative isolated hVEGFR-3D1-R2D2+3/hIgG1Fc clone (clone #2). (SEQ ID NO: 124 and SEQ ID NO: 125).

In addition to the above chimeric construct, a chimeric VEGF receptor/hIgG1Fc fusion protein was constructed having the first Ig-like domain of VEGFR-3, the second Ig-like domain of VEGFR-2 and the third Ig-like domain of VEGFR-1. The construct is designated R-3D1-R2D2-R1D3/hIgG1Fc.

To clone the pIgPlus-hVEGFR-3D1-R2D2-R1D3/hIgG1Fc construct, PCR was performed using pIgPlus-hVEGFR-3D1-R2D2+3/hIgG1Fc as a template and the T7 forward and reverse primer v362 (5′-TACAATTGAGGACAAGCGTATGTCCACGAAGTAGTTTAACTGGACGAGGCGTGCTTATTTGCACAT CATAAATCCTATACC-3′) (SEQ ID NO: 126). The PCR-product was cut with HindIII and MfeI/MunI. The 787 bp VEGFR-3D1-R2D2+3/hIgG1Fc insert was ligated into the HindlIl- and partially MfeI-cut vector plgPlus-hVEGFR-1D1-3-IgG1Fc. The presence and sequence of the correct chimeric insert was confirmed by sequencing the a representative hVEGFR-3D1-R2D2-R1D3/hIgG1Fc clone (clone #6) (SEQ ID NO: 127 and SEQ ID NO: 128).

### Expression of Chimeric VEGFR/hIgG1Fc Fusions:

For expression analysis, the two new chimeric VEGF receptors and control constructs expressing R-1D1-3/hIgG1Fc, R-2D1-3/hIgG1Fc, R-3D1-3/hIgG1Fc, mature VEGF-C and VEGF-A165 were transiently transfected into 293T cells using JetPEI (QBioGene/MP Biomedicals, Irvine, CA). Metabolic labeling with ³⁵S-methionine and ³⁵S-cysteine was carried out at 48 hours post-transfection and labeling maintained for 24 hours. The serum-free conditioned medium was then immunoprecipitated using Protein A sepharose and either: a) specific antiserum against human mature VEGF-C; b) goat polyclonal antibody against human VEGF-A (R&D systems, Minneapolis, MN); or, c) serum-free medium of 293T cells taken 48 to 72 hours post-transient transfection with VEGF receptor/hIgG1Fc proteins (control proteins, R-1D1-3, R-2D1-3, R-3D1-3; chimeric proteins, R-3D1-R2D2+3 and R-3D1-R2D2-R1D3).

The immunoprecipitated fractions were analyzed on 17% SDS-PAGE and the dried gels were exposed for 12 hours on phosphoimager plates or 36 hours on X-ray films. Expression analysis demonstrated that the chimeric receptor fusion proteins exhibited high expression levels in transfected 293 T cells.

### Analysis of Binding Properties of Chimeric VEGF Receptor/hIgG1Fc Fusions:

Ligand binding analysis was performed as described for the VEGF-C/VEGF-A hybrid growth factors in Example 1. Briefly, the unlabeled conditioned medium of transiently transfected 293T cells expressing the chimeric VEGFR/IgG1Fc fusion proteins was used to precipitate the ³⁵S metabolically labeled mature VEGF-C, full-length VEGF-C, and VEGF-A₁₆₅. SDS-PAGE of ligands immunoprecipitated with chimeric and control VEGFR/IgFc showed that the R-3D1-R2D2-R1D3/Ig chimeric protein strongly bound both VEGF-A and VEGF-C, as predicted based on the VEGFR2 and R1 immunoglobulin domains. In one experiment, the chimeric construct R-3D1-R2D2+3/Ig exhibited binding to VEGF-C and not VEGF-A. A second experiment with the R-3D1-R2D2+3/Ig construct showed only weak binding to VEGF-A.

These results demonstrate that the ligand binding constructs generated herein are useful in developing compositions that bind multiple growth factors involved in numerous cell activities. These constructs provide promising therapy for diseases such as cancer and other proliferative diseases wherein multiple growth factors mediate the condition or disease state.

### Example 4

### Assay for Neutralization of Growth Factor Activity

The following protocol provides an assay to determine whether a binding construct neutralizes one or more PDGF/VEGF growth factors by preventing the growth factor(s) from stimulating phosphorylation of its receptor.

Cells such as NIH 3T3 cells are transformed or transfected with a cDNA encoding a PDGFR/VEGFR receptor, such as VEGFR-3, and cultured under conditions where the encoded receptor is expressed on the surface of the cells. Transfected cells are cultured with either 1) plain growth medium; 2) growth medium supplemented with 50 ng/ml of one or more ligands for the recombinant receptor, such as fully processed VEGF-C and/or VEGF-D, which are ligands for VEGFR-3; 3) growth medium supplemented with 50 ng/ml of growth factor that does not bind the recombinant receptor (e.g., VEGF-A in the case of VEGFR-3), to serve as a control; or any of (1), (2), or (3) that is first pre-incubated with varying concentrations of a binding construct to be tested.

After culturing with the culture mediums described above in the presence or absence of the binding construct, the cells are lysed, immunoprecipitated using anti-receptor (e.g., anti-VEGFR-3) antiserum, and analyzed by Western blotting using anti-phosphotyrosine antibodies. Cells stimulated with the appropriate growth factor ligand (VEGF-C/D) stimulate VEGFR-3 autophosphorylation, which is detected with the anti-phosphotyrosine antibodies. Binding constructs that reduce or eliminate the ligand-mediated stimulation of receptor phosphorylation (e.g., in a dose-dependent manner) are considered neutralizing binding constructs.

### Example 5

### EPO Chimera Survival/Proliferation Blocking Assay

A binding construct is tested for the ability to block the binding of the growth factor(s) to their receptors, using bioassays of receptor binding and cross-linking. These assays involve the use of Ba/F3 pre-B cells which have been transfected with plasmid constructs encoding chimeric receptors consisting of the extracellular domain of growth factor receptors and the cytoplasmic domain of the erythropoietin receptor (Stacker, S A. et al., J. Biol. Chem. 274:34884-34892, 1999; Achen, M G. et al., Eur. J. Biochem. 267:2505-2515, 2000). These cells are routinely passaged in interleukin-3 (IL-3) and will die in the absence of IL-3. However, if signaling is induced from the cytoplasmic domain of the chimeric receptors, these cells survive and proliferate in the absence of IL-3. Such signaling is induced by ligands which bind and cross-link the extracellular domains of the chimeric receptors. Therefore binding of a growth factor ligand to the extracellular domains of the chimeric receptors causes the cells to survive and proliferate in the absence of IL-3. Addition of binding constructs that block the binding of growth factor to the extracellular domains will cause cell death in the absence of IL-3. An alternative Ba/F3 cell line which expresses a chimeric receptor containing the extracellular domain of the Tie2 receptor (that does not bind VEGF family members) is not induced by the relevant growth factors to proliferate and is used, in the presence of IL-3, as a control to test for non-specific effects of potential inhibitors.

In an exemplary assay, a binding construct that can bind VEGF-A and VEGF-C is tested. Samples of purified VEGF-A and VEGF-C are incubated with varying amounts of the binding construct for one hour at 4° C. in PBS before dilution of the mixtures 1:10 with IL-3-deficient cell culture medium. Ba/F3 cell lines expressing receptor(s) capable of binding the growth factors are then incubated in the media for 48 hours at 37° C. To measure DNA synthesis in th cells, 1 μCi of 3H-thymidine is added and the cells are incubated for 4 hours prior to harvesting. Incorporated 3H-thymidine is measured using a cell harvester (Tomtec®) and beta counting. The ability of the binding construct to block growth factor-mediated cell growth and survival (as measured by DNA synthesis) is analyzed relative to the control Tie2 cell line in the presence of IL-3. Growth inhibition in the experimental group relative to the control group demonstrates that the binding construct blocks cell growth, presumably by blocking the binding and cross-linking of receptors by growth factor ligands at the cell surface.

### Example 6

### Effect of Binding Constructs on BCE Migration

Solutions containing growth factors pre-incubated alone or with varying concentrations of a binding construct are placed in wells made in collagen gel and used to stimulate the migration of bovine capillary endothelial (BCE) cells in the gel as follows. A further control comprising neither growth factor ligand nor binding construct may also be employed, as may a control with just binding construct. Binding constructs that cause a decrease in migration (relative to when growth factor alone is employed) have an indication as therapeutics to prevent or retard angiogenesis.

BCE cells (Folkman et al., Proc. Natl. Acad. Sci. (USA), 76:5217-5221 (1979)) are cultured as described in Pertovaara et al., J. Biol. Chem., 269:6271-74 (1994). These or other cells employed may be transformed with growth factor receptor if not already expressed. For testing of VEGF-A/VEGF-C binding constructs, cells would be transformed with both VEGFR-2 and/or VEGFR-3. The collagen gels are prepared by mixing type I collagen stock solution (5 mg/ml in 1 mM HCl) with an equal volume of 2× MEM and 2 volumes of MEM containing 10% newborn calf serum to give a final collagen concentration of 1.25 mg/ml. The tissue culture plates (5 cm diameter) are coated with about 1 mm thick layer of the solution, which is allowed to polymerize at 37° C. BCE cells were seeded on top of this layer. For the migration assays, the cells are allowed to attach inside a plastic ring (1 cm diameter) placed on top of the first collagen layer. After 30 minutes, the ring is removed and unattached cells are rinsed away. A second layer of collagen and a layer of growth medium (5% newborn calf serum (NCS)), solidified by 0.75% low melting point agar (FMC BioProducts, Rockland, ME), are added. A well (3 mm diameter) is punched through all the layers on both sides of the cell spot at a distance of 4 mm, and the sample or control solutions are pipetted daily into the wells. Photomicrographs of the cells migrating out from the spot edge are taken after six days through an Olympus CK 2 inverted microscope equipped with phase-contrast optics. The migrating cells are counted after nuclear staining with the fluorescent dye bisbenzimide (1 mg/ml, Hoechst 33258, Sigma).

The number of cells migrating at different distances from the original area of attachment towards wells containing sample solutions are determined 6 days after addition of the media. The number of cells migrating out from the original ring of attachment is counted in five adjacent 0.5 mm×0.5 mm squares using a microscope ocular lens grid and 10× magnification with a fluorescence microscope. Cells migrating further than 0.5 mm are counted in a similar way by moving the grid in 0.5 mm steps. The experiments are carried out twice with similar results. Daily addition of 1 ng of FGF2 into the wells may be employed as a positive control for cell migration.

### Example 7

### Suppression of VEGFR-3 Signalling Pathway Improves Allograft Survival

Experiments described herein elucidate the role of lymphatic vessels and their principal growth signalling pathway, VEGF-C/VEGFR-3, in experimental cardiac allograft alloimmunity and arteriosclerosis. We found functional lymphatic vessels in rat cardiac allografts that co-expressed LYVE-1, Prox-1, VEGFR-3 and chemokine CCL21, and were active in transferring antigen presenting cells (APC). Chronic rejection enhanced the number of graft-infiltrating VEGF-C⁺ inflammatory cells, and induced myocardial lymphangiogenesis. Lymphatic EC almost exclusively originated from donor-derived cells. Systemic VEGFR-3 inhibition with VEGFR-3-Ig gene delivery reduced allograft CCL21 production, alloimmune activation, and improved cardiac allograft survival of recipients receiving suboptimal cyclosporine A immunosuppression. In a mouse chronic rejection model, treatment with neutralizing VEGFR-3 antibodies reduced allograft CCL21 production, inflammation and arteriosclerosis. Collectively, our results indicate interplay of inflammation and lymphangiogenesis in cardiac allografts. Moreover, VEGFR-3 inhibition reduced APC trafficking possibly through direct DC-mediated and indirect CCL21 mediated effects. VEGFR-3 inhibition may thus be used as a novel non-T cell-targeted induction therapy to regulate alloimmune activation after solid organ transplantation.

### Methods

### Experimental Design

The effect of heart transplantation on the expression of lymphatic endothelial markers and lymphatic growth factors was investigated using a rat heterotopic heart transplantation model comparing non-transplanted hearts, acutely and chronically-rejecting cardiac allografts, and syngenic controls. Marker gene transgenic mice were used to determine the origin of VEGFR-3⁺ lymphatic EC in chronically-rejecting mouse cardiac allografts. The functional role of VEGFR-3 singalling in alloimmune responses was investigated by perfusing rat cardiac allograft recipients intrapotally with an adenovirally expressed soluble VEGFR-3 receptor extracellular domain (Ad.VEGFR-3-Ig) that traps VEGFR-3 ligands. Neutralizing monoclonal VEGFR-3 antibodies (VEGFR-3 mAb) were used to confirm the effect of VEGFR-3 inhibition on lymphangiogenesis and inflammation-driven arteriosclerosis in chronically-rejecting mouse cardiac allografts. Permission for animal experimentation was obtained from the State Provincial Office of Southern Finland. The mice and rats received care in compliance with the “Guide for the Care and Use of Laboratory Animals” prepared by the National Academy of Sciences and published by the National Academy Press (ISBN 0-309-05377-3, revised 1996).

### Rat and Mouse Heterotopic Heart Transplantation Model

Specific pathogen-free inbred male Dark Agouti (DA, RT1av1) and Wistar Furth (WF, RT1u) rats (Scanbur, Sollentuna, Sweden) weighing 250-300 g and 2-3 months of age were used. Heterotopic cardiac allografts were transplanted in abdominal position between fully MHC-mismatched strains. The donor heart was perfused through the inferior vena cava with 1 ml of +4° C. 0.9% NaCl with 500 IU heparin. The inferior and superior vena cava, and pulmonary veins were ligated. The ascending aorta and pulmonary artery were excised distally, and the donor heart was removed and kept in +4° C. PBS. The allograft aorta and pulmonary artery were then anastomozed to the abdominal aorta and vena cava inferior of the recipient. In the acute rejection model, no immunosuppression was used and the syngrafts (DA->DA) and allografts (DA->WF) were harvested at 5 days. In the chronic rejection model, the recipients received cyclosporine A (CsA, Novartis Basel, Switzerland) 2 mg/kg/d for the first week and 1 mg/kg/d thereafter, and the grafts were harvested at 8 weeks. CsA was dissolved in Intralipid (100 mg/ml, Fresenius Kabi, Bad Homburg, Germany) and was administered subcutaneously.

In the mouse model, specific pathogen-free inbred male BALB/c (B/c, H-2d) and C57BL/6J (B6, H-2b) mice (Harlan) weighing 25-30 g and 2-3 months of age were used. The recipients received FK506 (intramuscular formulation, Astellas Pharma, Tokyo, Japan) subcutaneously 3.0 mg/kg/d for the first week and 1.5 mg/kg/d thereafter as background immunosuppression, and the allografts were harvested at 8 weeks for histological and immunohistochemical analysis. This immunosuppression was chosen after preliminary studies with different FK506 dosing, as the current administration resulted in prolonged allograft survival and development of CAV.

### Origin of Allograft Lymphatic EC

Transgenic marker gene mice that express LacZ under VEGFR-3 promoter (VEGFR-3/LacZ) were used to investigate the origin of allograft VEGFR-3 lymphatic EC. To investigate recipient-derived VEGFR-3 expression in the transplanted heart, Balb/c hearts were transplanted to VEGFR-3/LacZ recipients using the mouse chronic rejection heart transplant model. Replacement of allograft lymphatic EC with bone marrow (BM)-derived cells was investigated using C57 mice that had received a BM transplant from GFP-expressing syngenic mice (GFP-BM) as Balb cardiac allograft recipients using the mouse chronic rejection heterotopic heart transplant model (n=3). Allografts were harvested at 8 weeks. Samples were first incubated in 2% paraformaldehyde for 30 min, then in 20% sucrose overnight, embedded in TissueTek and snap-frozen in liquid nitrogen.

### Effect of VEGFR-3 Inhibition on Alloimmune Responses in Rat Cardiac Allografts

To investigate the role of VEGFR-3 ligand inhibition in rat cardiac allografts, recipients were perfused in the beginning of the operation intraportally with adenoviral vectors (1×10⁹ pfu in 1 ml) encoding control vector (Ad.LacZ or Ad.GFP) or soluble VEGFR-3 receptor (Ad.VEGFR-3-Ig). The recipients received CsA 1.0 mg/kg/d as background immunosuppression. The recipient livers and the transplanted allografts were harvested on day 5 (ad.GFP. n=7; ad.VEGFR-3-Ig n=7) to investigate the efficiency of adenoviral gene transfer, and alloimmune activation in the allograft. The effect of intraportal ad.VEGFR3-Ig perfusion on cardiac allograft survival (ad.LacZ, n=10; ad.VEGFR-3-Ig, n=10) was investigated by harvesting allografts at 8 weeks or if the graft function deteriorated. Hepatocyte GFP expression was detected immunohistochemically.

### ELISA

ELISA (Quantikine-R&D Systems) was used to detect the presence of VEGFR-3-Ig in rat serum collected at day 5 postransplantation, confirming the functionality of the adenoviral gene transfer in our system.

### Effect of VEGFR-3 Inhibition in Chronically-Rejecting Mouse Cardiac Allografts

To investigate the functional role of VEGFR-3, mouse cardiac allograft recipients were treated with 800 μg of rat IgG (n=7; Sigma-Aldrich, St. Louise, MO) or rat anti-mouse VEGFR-3 neutralizing antibody (n=8; mF4-31C1, ImClone, New York, NY). Antibodies were administered intraperitoneally every third day for four weeks, starting immediately after operation.

### Histology

Cardiac transplant arteriosclerosis was determined by two independent observers in blinded manner from paraformaldehyde-fixed paraffin sections stained with hematoxylin-eosin and Resorcin fuchsin for internal elastic lamina using computer-assisted image processing (Axiovision 4.4, Carl Zeiss, Oberkochen, Germany) and measuring the area surrounded by the internal elastic lamina and vessel lumen. Arterial occlusion percentage was determined as the ratio of neointimal area and internal elastic lamina area.

### Immunohistochemistry

Cryostat sections were stained using peroxidase ABC method (Vectastain Elite ABC Kit; Vector Laboratories, Burlingame, Calif.) and the reaction was revealed by 3-amino-9-ethylcarbazole (AEC, Vectastain). Immunofluorescense double stainings were performed using a sequential approach and Alexa Fluor 488 (green) and Alexa Fluor 568 (red), (Promega, Madison, WI) secondary antibodies. Antibodies and dilutions used were CD4 (5 μg/ml, 22021D), CD8 (5 μg/ml, 22071D), ED1 (5 μg/ml, 22451D), CD11$ (5 μg/ml, 553308) and IL-2Ra (5 μg/ml, 22090D) from BDPharmingen, San Diego, CA; Ki67 (1:2000, NCL-Ki67p) from Novocastra Laboratories Ltd, New Castle, UK; rabbit anti-mouse affinity purified LYVE-1 (1:1000 with TSA amplification) and Anti-mouse CCL-21/6Ckine Antibody (1:200) from Professor Kari Alitalo; VEGF-C(0.5 μg/ml, ab9546) and anti-GFP (1:200, ab 290) from Abcam, Cambridge, UK; mouse anti-rat OX-62 (10 μg/ml, MCA1029G), RECA-1 (50 μg/ml, MCA970) and major histocompatibility complex (MHC) class 11 (10 μg/ml, MCA46R) from Serotec, Oxford, UK; VEGFR-3 (200 μg/ml, AF743) from R&D Systems, Minneapolis, MN; PROX-1 (0.01 mg/ml, DP 3501P) from Acris Antibodies, Hiddenhausen, Germany. All analyses were performed in a blinded manner by two independent observers.

### Analysis of the Immunohistochemical Stainings

Graft-infiltrating inflammatory cells and LYVE-1+, VEGFR-3+ or CCL-21⁺ lymphatic vessels with clear lumen were counted from four random fields from each quadrant of the section's parenchyme with 40× magnification and are given as the mean number of positive cells or vessels per mm2. Lymphatic vessels were also counted from the epicardium of the section and the amount of vessels are given as the mean number of positive vessels per mm².

### RNA Isolation and Reverse Transcription

Total RNA was extracted using RNeasy Mini Kit (Qiagen, Hilden, Germany) (n=4-6 per group). Reverse transcription of mRNA was carried out from 100 ng total RNA in a final volume of 20 μl, using 200 U M-MLV reverse transcriptase (Sigma-Aldrich), with 20 U recombinant RNasin ribonuclease inhibitor (Promega), 0.5 mM dNTPs (Sigma-Aldrich), and 2.5 μM random nonamers (Sigma-Aldrich). After RT, 40 μl of nuclease-free water was added to each cDNA and 3 μl of each sample was used in each subsequent PCR reaction.

### Real-Time PCR

External standards were used to generate a standard curve for each gene of interest. The templates of these standards consisted of PCR fragments generated with the same primers as used in real-time PCR. The DNA concentrations were determined by spectrophotometry (Eppendorf, Hamburg, Germany), followed by calculation of the PCR fragment concentrations. For each standard curve, 10-fold serial dilutions were made starting from 10⁷ PCR fragments. The number of copies of the gene of interest was calculated from the corresponding standard curve using LightCycler software (Roche, Basel, Switzerland).

Real-time RT-PCR reactions were carried out in a LightCycler using LightCycler FastStart DNA MasterPLUS SYBR Green I mix (Roche), primer concentrations of 0.4 μM, and a cDNA amount corresponding to 5 ng total RNA in a reaction volume of 10 μl. A typical protocol included a 10-min denaturation step at +95° C. followed by 35 cycles with a +95° C. denaturation step for 10 sec, annealing at +59° C. for 10 sec, and extension at +72° C. depending on the length of the product (1 sec for 25 bp). Measurement of the PCR product was performed at the end of each extension period. Amplification specificity was checked using melting curve analysis. Results are given in relation to 18S rRNA molecule numbers.

The following primers for rat IL-2 (Gene Bank accession no. NM_053836), IL-4 (acc. No. NM_201270), IL-6 (acc. No. NM_012589), IL-10 (acc. No. NM_012854), TNF-α (acc. No. NM_012675), IFN-γ (acc. No. NM_138880), CCL-21 (acc. No. NM_011124) and FOXP-3 (acc. No. XM_228771) were used:

IL-2
fwd
(SEQ ID NO: 129)
5′-CTGAGAGGGATCGATAATTACAAGA-3′;
bwd
(SEQ ID NO: 130)
5′-ATTGGCACTCAAATTTGTTTTCAG-3′;
IL-4
fwd
(SEQ ID NO: 131)
5′-ATGTTTGTACCAGACGTCCTTACG-3′;
bwd
(SEQ ID NO: 132)
5′-TGCGAAGCACCCTGGAA-3′;
IL-6
fwd
(SEQ ID NO: 133)
5′-CCCAACTTCCAATGCTCTCCTAATG-3′;
bwd
(SEQ ID NO: 134)
5′-GCACACTAGGTTTGCCGAGTAGACC-3′;
IL-10
fwd
(SEQ ID NO: 135)
5′-TAAGGGTTACTTGGGTTGCC-3′;
bwd
(SEQ ID NO: 136)
5′-TATCCAGAGGGTCTTCAGC-3′;
TNF-α
fwd
(SEQ ID NO: 137)
5′-CTGTGCCTCAGCCTCTTCTCATTC-3′;
bwd
(SEQ ID NO: 138)
5′-TTGGGAACTTCTCCTCCTTGTTGG-3′;
IFN-γ
fwd
(SEQ ID NO: 139)
5′-GAGGTGAACAACCCACAGA-3′;
bwd
(SEQ ID NO: 140)
5′-TATTGGCACACTCTCTACCC-3′;
CCL-21
fwd
(SEQ ID NO: 141)
5′-CCCTGGACCCAAGGCAGT-3′;
bwd
(SEQ ID NO: 142)
5′-AGGCTTAGAGTGCTTCCGGG-3′;
and
FOXP-3
fwd
(SEQ ID NO: 143)
5′-GCTTGTTTGCTGTGCGGAGAC-3′;
bwd
(SEQ ID NO: 144)
5′-GTTTCTGAAGTAGGCGAACAT-3′.

### Flow Cytometry

The cardiac allograft spleens were harvested at 5 days after the transplantation to RPMI-1640 medium. The tissue was homogenized with a scalpel and 1×10⁶ spleen cells were incubated with FITC- or PE-conjugated antibodies for 15 minutes at room temperature. The cells were then washed twice with PBS and analyzed with a FACScan (Becton Dickinson) flow cytometer. Antibodies used were CD45-FITC (MCA43FT, Serotec), CD68-RPE (MCA341PE, Serotec), OX62-RPE (MCA1029PE, Serotec). IgG1-FITC (MCA43FT, Serotec) and IgG-RPE (MCA1209PE) were used as negative isotype controls.

### Statistics

All data are given as mean±SEM and analyzed by parametric Student T test, or by log-rank test (graft survival) using SPSS for Windows version 11.5.1 (SPSS inc., Chicago, IL). P<0.05 was regarded as statistically significant.

### Results

### Chronic Alloimmune Stimulus Induces Myocardial Lymphangiogenesis in Cardiac Allografts

As lymphatic growth is often seen during inflammation, we evaluated whether acute or chronic rejection induces lymphangiogenesis in rat cardiac allografts by using lymphatic endothelium-specific hyaluronan acid receptor-1 (LYVE-1). In the acute rejection model, fully MHC-mismatched rat heterotopic cardiac allograft recipients were non-immunosuppressed, and allografts developed an intense acute rejection at 5 days. In the chronic rejection model, allograft recipients received suboptimal cyclosporine A (CsA) immunosuppression, and allografts showed chronic rejection with moderate allograft inflammation, myocardial fibrosis, and arteriosclerosis at 8 weeks. We found small and large LYVE-1⁺ vessel structures in the myocardium of normal non-transplanted and transplanted hearts. These vessels opened to larger epicardial collecting LYVE-1⁺ lymphatic vessels. In normal hearts and syngeneic controls, LYVE-1⁺ lymphatic vessel density was two times higher in the epicardial area than in the myocardium. Chronic rejection doubled the myocardial LYVE-1⁺ lymphatic vessel density, suggesting active lymphangiogenesis during chronic allograft inflammation. Acute rejection decreased the epicardial lymphatic vessel density, possibly indicating lymphatic vessel destruction during intense inflammation.

Immunofluorescence double stainings of chronically-rejecting cardiac allografts demonstrated the expression of lymphatic endothelial cell transcription factor Prox-1 in the nucleus of the LYVE-1⁺ cells, confirming the lymphatic phenotype. This was further supported by the observation that LYVE-1 and rat vascular endothelial cell antigen −1 (RECA-1) were not expressed in same vessels, suggesting that these are markers of lymphatic and vascular EC in the rat, respectively. The proliferation marker Ki67 was infrequently found in LYVE-1⁺ EC, whereas several Ki67⁺ LYVE-1⁻ allograft-infiltrating mononuclear cells were detected outside LYVE-1⁺ lymphatic vessels. CD4⁺ and CD8⁺ T lymphocytes were mainly detected outside the LYVE-1⁺ lymphatic vessels. In contrast, ED1V macrophages and OX-62⁺ DC were found both outside and inside the LYVE-1⁺ vascular structures, indicating that the lymphatic vessels in cardiac allografts are functional in transferring APC.

### Macrophages and CD4⁺ Lymphocytes are the Major Source of VEGF-C in Chronically-Rejecting Cardiac Allografts

Our finding that chronic alloimune stimulus induces lymphangiogenesis in cardiac allografts prompted us to investigate the expression of a potent lymphangiogenic cytokine VEGF-C in transplanted hearts. VEGF-C was mainly expressed in graft-infiltrating mononuclear cells. The density of VEGF-C⁺ cells was similar in non-transplanted hearts, acutely rejecting cardiac allografts, and syngenic controls, whereas myocardial VEGF-C⁺density was two times higher in cardiac allografts undergoing chronic rejection. Immunofluorescense double stainings showed that a subset of ED1⁺ macrophages and CD4⁺ lymphocytes were VEGF-C positive, whereas CD8⁺ lymphocytes did not show VEGF-C immunoreactivity. These findings indicate that during chronic cardiac allograft rejection, macrophages and CD4⁺ T lymphocytes are the major source for lymphangiogenic VEGF-C.

### VEGFR-3 is Expressed in Lymphatic Endothelium and A Subset of Dendritic Cells in Cardiac Allografts

We determined the expression of VEGFR-3—the receptor for VEGF-C—in transplanted rat hearts. VEGFR-3 immunoreactivity was detected in lymphatic-like vessels of non-transplanted and transplanted hearts. Mononuclear cells were encountered inside the VEGFR-3⁺ vessels of cardiac allografts. Immunohistochemical staining of consecutive sections showed that VEGFR-3 and CCL21 expression was localized in the same lymphatic EC of chronically rejecting allografts. The density of VEGFR-3⁺ vessels was generally lower in the myocardium than in the epicardial area. Myocardial VEGFR-3⁺ vessel density was three times higher in chronically-rejecting cardiac allografts than in syngenic controls.

Immunofluorescence double stainings of chronically-rejecting cardiac allografts showed that endothelial VEGFR-3 imunoreactivity co-localized with LYVE-1 expression, although not all LYVE-1⁺ vessels expressed VEGFR-3. In addition to the lymphatic VEGFR-3 expression, we also detected lower VEGFR-3 immunoreactivity in occasional allograft-infiltrating mononuclear cells. The majority of these VEGFR-3⁺ cells were identified as OX-62⁺ DC, whereas very few ED1⁺ macrophages, and no CD4⁺ or CD8⁺ cells expressed VEGFR-3. ED1⁺ macrophages were often encountered inside the VEGFR-3⁺ lymphatic vessels. Collectively, these findings indicate that VEGFR-3 is expressed in lymphatic vessels and subset of DC in cardiac allografts.

Cardiac Allograft VEGFR-3⁺ Lymphatic Vessels but not Spleen Mariginal Zone VEGFR-3⁺ Vessels Produce CCL21

Distinct molecular properties of lymphatic endothelial cells, such as production of CCL21 chemokine (Kriehuber et al., J. Exp. Med., 194:797-808, 2001), are essential in the specialized function of lymphatic vessels in transferring APCs to secondary lymphoid organs. This prompted us to investigate whether CCL21 is produced by cardiac allograft lymphatic vessels. Immunohistochemical staining of consecutive sections showed that VEGFR-3 and CCL21 were expressed in the same lymphatic vessels of chronically-rejecting allografts. In contrast, VEGFR-3 and CCL21 expression did not co-localize in normal spleen or in the spleen of cardiac allograft recipients. Results indicated that VEGFR-3 was expressed in the endothelium of vessel-like structures around spleen T cell zones, whereas CCL21 expression localized to white pulp stromal cells and to the central arterioles. The finding that allograft VEGFR-3⁺ lymphatic vessels produce CCL21 suggests that lymphatic endothelial cell-derived chemokine-mediated signals are present at the exit of APCs from cardiac allografts similarly as in corneal allografts (Jin et al., Mol. Vis., 13:626-634, 2007). In contrast, the VEGFR-3⁺ vessels in the spleen (presumably capillaries or venous sinuses of the marginal zone) did not produce CCL21 but may be involved in leukocyte trafficking through non-CCL21-mediated mechanisms.

### The Majority of VEGFR-3⁺ Lymphatic EC in Chronically-Rejecting Mouse Cardiac Allografts are Donor-Derived

Recent evidence suggests that BM-derived and non-BM-derived VEGFR-3⁺ cells contribute to lymphangiogenesis (Kerjaschki et al., (2006), “Lymphatic endothelial progenitor cells contribute to de novo lymphangiogenesis in human renal transplants,” Nat. Med. 12: 230-234; and Maruyama et al., (2005), “Inflammation-induced lymphangiogenesis in the cornea arises from CD11b-positive macrophages,” J. Clin. Invest., 115: 2363-2372). As we found active lymphangiogenesis in chronically-rejecting cardiac allografts, we next used marker gene mice as cardiac allograft recipients to determine the origin of VEGFR-3⁺ lymphatic EC. First, C57/bl mice that had received BM transplantation from GFP mice (GFP-BM) were used as heart transplant recipients allowing the detection of BM-derived cells in cardiac allografts. The recipients were treated with suboptimal FK506 immunosuppression to prevent severe acute rejection, and the cardiac allografts were harvested eight weeks after the transplantation. Immunofluorescence double stainings showed that BM-derived GFP⁺ cells localized mainly around VEGFR-3⁺ lymphatic vessels. Less than 4% of the BM-derived GFP⁺ cells co-localized with VEGFR-3⁺ lymphatic EC.

As some of these GFP⁺ cells may actually be BM-derived inflammatory cells migrating to the VEGFR-3⁺ lymphatic vessels, Balb/c hearts were next transplanted to mice that express LacZ under VEGFR-3 promoter (VEGFR-3/LacZ, C57/bl background, n=3), to allow the direct detection of recipient-derived VEGFR-3⁺ lymphatic cells in the allografts. The x-gal staining revealed epicardial lymphatic endothelial VEGFR-3 expression in the VEGFR-3/LacZ recipient's own heart. In contrast, no recipient-derived VEGFR-3⁺ lymphatic EC were encountered in the myocardium of wild type cardiac allografts transplanted to VEGFR-3/LacZ recipients. These results indicate that the replacement of cardiac allograft VEGFR-3⁺ lymphatic EC with recipient BM-derived, or non-BM-derived cells is rare in this chronic rejection heterotopic heart transplantation model.

### VEGFR-3 Inhibition Improves Cardiac Allograft Survival

We next determined the effect of VEGFR-3 inhibition on alloimmune response by injecting suboptimally immunosuppressed rat cardiac allograft recipients intraportally with adenovirus vector encoding the soluble form of VEGFR-3 (Ad.VEGFR-3-Ig, VEGF-C/D-trap). Hepatocyte GFP expression was seen in Ad.GFP-perfused recipients, but not in Ad.VEGFR-3-Ig-perfused recipients. See FIGS. 1A and 1B. Also, the serum levels of VEGFR-3-Ig were elevated in the Ad.VEGFR-3-Ig-perfused animals 5 days after transplantation (FIG. 1C), and remained elevated for at least 21 days, confirming the functionality of the adenoviral gene transfer.

Next, we investigated the effect of VEGFR-3 inhibition on the survival of fully MHC-mismatched rat cardiac allografts. In non-immunosuppressed cardiac allograft recipients, intraportal Ad.VEGFR-3-Ig-perfusion increased allograft survival from 4.9 to 6.0 days (p<0.05, n=7 per group). Intraportal Ad.VEGFR-3-Ig-perfusion in cardiac allograft recipients receiving suboptimal dose of CsA significantly improved allograft survival. See FIG. 1D. Our results thus suggest that VEGFR-3 inhibition together with CsA background immunosuppression markedly prolongs long-term allograft survival while it only has a marginal effect as sole treatment in a fully MHC-mismatched model.

### VEGFR-3 Inhibition Decreases Cardiac Intragraft CCL21 Production, Alloimmune Activation and Effector Cell Recruitment

To clarify the underlying mechanisms behind the beneficial effect on allograft survival, we used the same experimental setting (Ad.VEGFR-3 perfusion and suboptimal CsA immunosuppression) and harvested the allografts 5 days after transplantation. Ad.VEGFR-3-Ig-perfusion decreased the density of, graft infiltrating CD8+ T cells, and alloimmune activation in the form of IL-2Rα⁺ and MHC class II expression. In contrast, no changes in graft infiltrating ED1⁺ macrophages, allograft CD4⁺ cells or OX-62⁺ DC s or the density of LYVE-1⁺ vessels were observed.

Real time RT-PCR showed that intraportal Ad.VEGFR-3-Ig perfusion resulted in a two-fold decrease in allograft CCL21 mRNA expression. No significant changes were observed in IL-6, Foxp3, INF-γ, IL-10, TNF-α, NFκβ, lymphotoxin (LT)-α or LT-β mRNA levels. Together, these findings indicate that VEGFR-3 inhibition decreases alloimmune activation and infiltration of effector cells in cardiac allografts together with a decrease in intragraft CCL21 production.

### VEGFR-3 Inhibition Decreases Dendritic Cell Recruitment to Spleen

We next investigated whether the reduction of alloimmune response with VEGFR-3 inhibition after heart transplantation was associated with impaired APC recruitment to recipient secondary lymphoid organs using FACS analysis from peripheral blood and spleen leukocytes. Ad.VEGFR-3-Ig perfusion decreased the proportion of OX-62⁺ DC, and ED1⁺ cells (p=NS) of spleen CD45⁺ leukocytes. The proportion of OX-62⁺ cells of peripheral blood leukocytes was 12.7±2.5% in Ad.GFP group and 15.7±4.2% in Ad.VEGFR-3 group. This indicates that the decrease in spleen DC was due to impaired APC homing to the spleen rather than impaired APC mobilization from the BM. Results further indicated that VEGFR-3-Ig increased the proportion of VEGFR-3⁺ leukocytes in peripheral blood possibly due to a trapping effect. VEGFR-3-Ig did not change the proportion of OX-62+DC in peripheral blood but decreased the recruitment of OX-62+DC to the recipient spleen. These results indicate that VEGFR-3 regulates APC traffic to secondary lymphoid organs.

### VEGFR-3 Inhibition Regulates Chemokine Balance Between Allograft and Secondary Lymphoid Organs

RT-PCR analysis of a subpopulation (n=3) revealed that VEGFR-3 inhibition was associated with over two-fold increase in spleen mRNA levels of CCL21, IL-10, and Foxp3. Spleen VEGFR-3 immunoreactivity was mainly detected in lymphatic-like vessel structures surrounding the T cell zones, whereas CCL21 immunoreactivity was mainly detected in the T cell zones and the central arterioles, and did not co-localize with VEGFR-3⁺ vessels. In cardiac allografts on the other hand, CCL21 was mainly expressed in VEGFR-3⁺ lymphatic-like vessels. These observations, together with the unexpected CCL21 response after VEGFR-3 inhibition, may indicate that CCL21 is differentially regulated in peripheral tissues and secondary lymphatic tissue.

We performed real time RT-PCR analysis of the recipient spleen 5 days after heart transplantation to investigate whether VEGFR-3-Ig decreased CCL21 production in the spleen similarly as in the allograft. In contrast to the results in the transplanted heart, treatment with VEGFR-3-Ig actually resulted in 1.5 times higher CCL21 mRNA levels in the spleen. We also observed that VEGFR-3-Ig markedly increased the ratio of spleen-to-allograft CCL21 mRNA from 9:1 to 23:1. The differential effect of VEGFR-3 inhibition on allograft and spleen CCL21 mRNA production may be explained by the finding that cardiac allograft VEGFR-3⁺ lymphatic vessels produced CCL21 whereas VEGFR-3 and CCL21 were not produced by the same cells in the spleen. Further RT-PCR analysis of the spleen revealed that VEGFR-3 inhibition resulted in a significant increase in Treg transcription factor Foxp3. This is interesting in the light of recent reports that CCL21 plays a vital role in homing, localization and function of Tregs (Schneider et al., J. Exp. Med., 204:735-745, 2007; Kocks et al., J. Exp. Med., 204:723-734, 2007). In addition, VEGFR-3 inhibition did not significantly alter spleen mRNA levels of IL-10 (Hori et al., Science, 299:1057-1061, 2003), IL-6, IFN-γ, TNF-α□, NF-κB, LT-α□, and LT-β at 5 days after transplantation. These results suggest that VEGFR-3 inhibition regulates chemokine balance between allograft and secondary lymphoid organs in favour of attenuated immune response.

### VEGFR-3 Neutralizing Monoclonal Antibody Decreases Inflammation and Inflammation-Driven Arteriosclerosis in Chronically-Rejecting Mouse Cardiac Allografts

We wanted to confirm the results of VEGFR-3 inhibition with neutralizing antibodies against VEGFR-3 in another chronic rejection heart transplantation model. Mouse cardiac allograft recipients received suboptimal FK506 immunosuppression to prevent intense acute rejection and to allow the development of chronic rejection at 8 weeks. In addition, the recipients were treated either with rat IgG or rat anti-mouse neutralizing antibodies (VEGFR-3 mAb, ImClone) against VEGFR-3 for one month. At two months, the effect of VEGFR-3 inhibition reduced the density of VEGFR-3⁺ and CCL-21⁺ lymphatic vessels in the allograft. In addition, VEGFR-3 inhibition reduced the density of CD4⁺ lymphocytes, CD8⁺ lymphocytes, and CD11b⁺ myelomonocytic cells. Interestingly, treatment with VEGFR-3 mAb significantly decreased the mean arterial occlusion compared to the control group. Our results thus show that early treatment with neutralizing VEGFR-3 antibody decreases allograft CCL21 production, inflammation, and arteriosclerosis in chronically-rejecting mouse cardiac allografts.

Finally, because lymphoid neogenesis (i.e., organization of chronic inflammatory infiltrates into functional ectopic germinal centers or tertiary lymphoid organs (TLOs)), has been linked with the formation of chronic rejection (Thaunat et al., Proc. Natl. Acad. Sci. USA, 102:14723-14728, 2005; Baddoura et al., Am. J. Transplant, 5:510-516, 2005; Nasr et al., Am. J. Transplant., 7:1071-1079, 2007), we investigated the presence of TLOs in our chronically-rejecting cardiac allografts. Immunohistochemical stainings revealed a characteristic pattern of peripheral node adressin-positive high endothelial venules, and discrete B and T cell accumulation only in one allograft in the control group (FIG. 10, A-C). This implies that lymphoid neogenesis is not a critical phenomenon at least in the two month end-point of our chronic rejection model, and the effect of VEGFR-3 inhibition on TLO formation thus cannot be evaluated in this model within this timeframe.

### Analysis

The lymphatic network is adapted at both structural and molecular level to transfer leukocytes out of tissues. The thin-walled lymphatic capillaries provide easy access for interstitial cells and fluid, whereas the smooth muscle coverage and valves of the collecting lymphatic vessels provide unilateral movement towards secondary lymphoid organs. Lymphatic EC have distinct molecular properties that reflect their function and have been utilized for the detection of lymphatic vessels. These cells express podoplanin, hyaluronan receptor LYVE-1, VEGFR-3 and inflammatory cytokine CCL21 that are not found in vascular EC. The identification of signals that regulate lymphatic growth—most importantly VEGF-C/VEGFR-3—has greatly improved our knowledge of lymphatic vessels in both physiological and pathological situations.

Effective transfer of APC from transplanted organs to secondary lymphoid organs is critical for the priming of alloreactive T cells and the development of alloimmune responses that may be detrimental for the heart transplant recipient. In the current study, we found that chronic cardiac allograft rejection increased myocardial lymphatic vessel density. The capillary lymphatics in cardiac allografts opened to epicardial collecting lymphatic vessels, and expressed the lymphatic transcription factor Prox-1, LYVE-1, VEGFR-3, and CCL21. In addition, APC such as macrophages and DC were often encountered inside these vessels, indicating vessel functionality. Active lymphangiogenesis is seen in human kidney transplants with nodular inflammatory infiltrates (Kerjaschki et al., (2004), “Lymphatic neoangiogenesis in human kidney transplants is associated with immunologically active lymphocytic infiltrates,” J. Am. Soc. Nephrol., 15: 603-612) and in other inflammatory conditions. As the observed lymphangiogenesis in chronically-rejecting allografts in this study was accompanied with an increase in VEGF-C-producing macrophages and CD4⁺ lymphocytes, our results suggest interplay of chronic inflammation, VEGF-C/VEGFR-3 signalling, and lymphangiogenesis in cardiac allografts.

Lymphatic EC in the transplanted heart may originate from recipient BM cells, from recipient non-BM cells or from donor cells. Recently, it was shown that recipient-derived lymphatic endothelial progenitor cells—possibly in the form of macrophages—participate in lymphangiogenesis of human kidney allografts (Kerjaschki et al., (2006), “Lymphatic endothelial progenitor cells contribute to de novo lymphangiogenesis in human renal transplants,” Nat. Med. 12: 230-234). Also, macrophages may directly trans-differentiate to lymphatic EC in the inflamed cornea (Maruyama et al., (2005), “Inflammation-induced lymphangiogenesis in the cornea arises from CD11b-positive macrophages,” J. Clin. Invest. 115: 2363-2372), and may provide cytokines for the expansion of resident lymphatics (Kerjaschki, D. (2005), “The crucial role of macrophages in lymphangiogenesis,” J. Clin. Invest. 115: 2316-2319). Here, we used marker gene mice as cardiac allograft recipients and found that recipient-derived cells contribute only minimally to the formation of VEGFR-3+ lymphatic vessels in the heterotopically transplanted hearts. As Kerjascki et al found that about 13% of Prox-1+ lymphatic vessels in rejected and nephrectomized kidney transplants originated from the recipient, it is possible that the involvement of recipient-derived lymphatic progenitors is dependent on the severity of allograft injury. Paralleling this hypothesis, the degree of allograft injury may determine whether allograft vascular EC originate from the recipient—as in aortic transplantation—or from the donor—as in cardiac allografts (Hillebrands et al., (2001), “Origin of neointimal endothelium and alpha-actin-positive smooth muscle cells in transplant arteriosclerosis, J. Clin. Invest., 107: 1411-1422). Also, the actual effect of lymphatic endothelium chimerism of transplanted organs on alloimmune responses remains unknown.

Chen et al (2004) have recently reported that VEGFR-3 inhibition impairs DC migration to draining LN and improves the survival of cornea transplants. These effects were possibly mediated through direct inhibition of VEGFR-3+DC migration independent of lymphangiogenic effects. However, the functional role of VEGFR-3 after solid organ transplantation has been unclear. Here, systemic VEGFR-3 inhibition using adenoviruses encoding soluble VEGFR-3-Ig that traps VEGFR-3 ligands decreased DC migration to spleen, alloimmune activation and improved the long term survival of cardiac allografts. Similarly, treatment with neutralizing VEGFR-3 antibodies decreased allograft inflammation and development of inflammation-driven arteriosclerosis in the chronically rejecting mice cardiac allografts. As we found VEGFR-3⁺ DC in cardiac allografts, it is possible that VEGFR-3 inhibition had direct effects on DC migration in the current study similar to the findings in the corneal transplantation model (18).

In addition to the direct effects on VEGFR-3+DC, our results suggest that VEGFR-3 inhibition also had lymphatic EC- and chemokine-mediated effects. Specifically, both VEGFR-3 and CCL21, a chemokine for CCR7+ APC, were co-expressed in allograft lymphatic EC, and VEGFR-3 inhibition decreased allograft CCL21 production. Our results thus indicate that VEGFR-3 in allograft lymphatic EC may regulate the production of CCL21, that may in turn facilitate the movement of APC from the allograft to secondary lymphoid tissue and subsequent alloimmune activation. Surprisingly, in contrast to the allograft, VEGFR-3 inhibition increased CCL21 production in the spleen. VEGFR-3 was mainly expressed around the spleen T cell zones whereas the central arterioles and stromal cells of the T cell zones were the main source of CCL21 in the spleen. Therefore, the regulation of CCL21 production may be different in the heart and the spleen due to the differential pattern of VEGFR-3 and CCL21 expression.

In experimental studies, the survival of cardiac allografts is modestly increased in CCR7-deficient recipients as well as in CCL21-deficient recipients (Forster et al., (1999), “CCR7 coordinates the primary immune response by establishing functional microenvironments in secondary lymphoid organs,” Cell, 99: 23-33; and Colvin et al., (2005), “CXCL9 antagonism further extends prolonged cardiac allograft survival in CCL19/CCL21-deficient mice,” Am. J. Transplant., 5: 2104-2113). Collectively, these studies show an important but not critical role of CCL21/CCR7-signalling in alloimmune reactions.

In the present study, VEGFR-3 inhibition that resulted in decreased CCL21 production did not completely prevent alloimmune responses in recipients receiving suboptimal dose of CsA. Therefore, VEGFR-3 inhibition alone may not completely prevent alloimmune responses in transplant recipients. For optimal results VEGFR-3 inhibition can be used as induction or adjuvant therapy in the prevention and treatment of acute rejection, in addition to (in combination with) conventional T-cell-targeted immunosuppression. Combination therapy targeting other VEGFR's or PDGFR's or their cognate growth factors also is contemplated. Interestingly, VEGFR-3 inhibition resulted in over two-fold increase in spleen Foxp3 and IL-10 mRNA production. Therefore, VEGFR-3 inhibition may also have beneficial effects on Treg, but further studies are needed to clarify the effect of VEGFR-3 signalling on Tregs.

In conclusion, these results indicate that VEGF-C/VEGFR-3 signalling has important effects on proximal events in cardiac allograft alloimmunity and inflammation-driven arteriosclerosis, possibly through regulating lymphatic endothelial cell CCL21 production and leukocyte trafficking and through direct effects on VEGFR-3⁺ DC. As an important safety aspect, adult lymphatic vessels are fairly resistant to VEGFR-3 inhibition, suggesting that this treatment in transplant recipients would also primarily inhibit lymphangiogenesis and the functionality of lymphatic vessels in contrast to regression of the existing lymphatic network. Therefore, VEGFR-3 inhibition could be used as a non-T cell-targeted induction therapy to regulate alloimmune activation after solid organ transplantation.

### Example 8

### VEGFR-1 and -2 Regulate Inflammation, Myocardial Angiogenesis and Arteriosclerosis in Chronically Rejecting Cardiac Allografts/R1

We investigated how the two vascular endothelial growth factor receptors VEGFR-1 and VEGFR-2 regulate inflammation and angiogenesis in chronically rejecting cardiac allografts. As described below in detail, chronic rejection in mouse cardiac allografts induced primitive myocardial, adventitial, and intimal angiogenesis with endothelial expression of CD31, stem cell marker c-kit, and VEGFR-2. Experiments using marker gene mice or rats as cardiac allograft recipients revealed that replacement of cardiac allograft endothelial cells with recipient bone-marrow- or non-bone-marrow-derived cells was rare and restricted only to sites with severe injury. Targeting VEGFR-1 with neutralizing antibodies in mice reduced allograft CD11b+ myelomonocyte infiltration and allograft arteriosclerosis. VEGFR-2 inhibition prevented myocardial c-kit+ and CD31⁺ angiogenesis in the allograft, and decreased allograft inflammation and arteriosclerosis. These results indicate an interplay of inflammation, primitive donor-derived myocardial angiogenesis, and arteriosclerosis in transplanted hearts, and further indicate that targeting VEGFR-1 and -2 with inhibitors differentially regulate these pathological reparative processes.

### Materials and Methods

### Experimental Design

Mouse chronic rejection heart transplantation model and immunohistochemical stainings were used to identify angiogenesis and progenitor cells in allografts. Marker gene mice and rats, and strain-specific major histocompatibility complex (MHC) class I antibodies, were used to determine whether allograft EC originate from the donor, or from the recipient. Neutralizing antibodies were used to investigate the functional role of VEGFR-1 and VEGFR-2 on mouse cardiac allograft angiogenesis, inflammation, and arteriosclerosis.

### Mouse Chronic Rejection Heterotopic Heart Transplantation Model

Heterotopic cardiac allografts were transplanted in abdominal position from Balb (B/c, H-2^{d}) to C57 (B6, H-2^{b}) mice (Harlan, Horst, The Netherlands). The recipients received sub-optimal FK506 immunosuppression (i.m. formulation, Astellas Pharma, Tokyo, Japan) and the allografts were harvested at 8 weeks.

### Origin of Allograft Endothelial Cells

Tie1/LacZ rats32 were used as allograft recipients (n=4) or donors (n=14, with or without immunosuppression) to investigate Tie1 expression, and the origin of Tie1-positive EC in transplanted hearts. Contribution of BM-derived cells in allograft angiogenesis was investigated using recipient mice with green fluorescent protein-expressing BM cells (GFP-BM, n=3). See Rajantie et al., “Adult bone marrow-derived cells recruited during angiogenesis comprise precursors for periendothelial vascular mural cells,” Blood, (2004); 104: 2084-2086.

### VEGFR-1 and VEGFR-2 Inhibition

Cardiac allograft recipients were treated with 800 μg of rat IgG (n=8; Sigma-Aldrich, St. Louis, MO), anti-VEGFR-1 antibody (n=9; MF1, ImClone, New York, NY), anti-VEGFR-2 antibody (n=9; DC101, ImClone) or their combination (n=10) every third day for 10 doses, starting immediately after the transplantation.

### Histology and Immunohistochemistry

Arterial occlusion percentage was determined using morphometry. Immunohistochemical stainings were performed using peroxidase ABC method or Alexa Fluor 488 (green) and 568 (red, Promega, Madison, WI) secondary antibodies.

### Analysis of Immunohistochemical Stainings

Allograft parenchymal inflammatory cells and c-kit+ capillaries were counted from 16 random sections, and are summarized as the mean density of positive cells or vessels. CD31 and α-SMA immunofluorescense stainings were analyzed with Axioplan 2 microscope and Axiovision 4.2 analysis software (Carl Zeiss, Oberkochen, Germany) using a semiautomated script.

### Real Time RT-PCR

Total RNA was extracted using RNeasy Mini Kit (Qiagen, Hilden, Germany) (n=4-6 per group). RT-PCR reactions were carried out using LightCycler (Roche, Basel, Switzerland) and the results are given in relation to 18S rRNA molecule numbers.

### Statistical Analysis

Data are mean±SEM and analyzed by parametric ANOVA with Dunnett's correction to compare the treatment groups to the control group. Linear regression analysis was applied to evaluate relation of c-kit+ cells to CD11b+ cells and to cardiac allograft vasculopathy (CAV). P<0.05 was regarded as statistically significant.

### Results

### Chronic Rejection Induces Primitive Myocardial Angiogenesis in Cardiac Allografts

We detected only occasional stem cell marker c-kit immunoreactive cells in cross-sections of non-transplanted mouse hearts. In contrast, numerous myocardial capillary-like c-kit+ cells and c-kit+ vein EC were observed in chronically-rejecting cardiac allografts harvested 2 months after the transplant operation. In allografts with severe arteriosclerotic changes, c-kit+ cells were also found in the adventitia and intima of coronary arteries.

Allograft myocardial c-kit+ cells were nearly all positive for endothelial marker CD31, and co-expressed VEGFR-2. The majority of c-kit+ capillaries did not express proliferation marker Ki67, but some c-kit+ cells with nuclear Ki67 immunoreactivity were also detected.

In contrast to the preferential expression of VEGFR-2 in the endothelium, VEGFR-1 was mainly expressed in allograft α-SMA+ SMC. In peripheral blood, over 50% of VEGFR-1⁺ cells coexpressed the myelomonocyte marker CD11b.

No specific immunoreactivity with IgG control was observed.

A positive correlation was verified between the density of c-kit+ capillaries in the myocardium and the number of allograft-infiltrating CD11b+ myelomonocytic inflammatory cells, as well as with the incidence of arteriosclerotic changes and the mean occlusion of allograft arteries. These results indicate that chronic rejection in transplanted hearts induces myocardial, adventitial and intimal angiogenesis with endothelial expression of primitive markers c-kit and VEGFR-2.

Endothelial Replacement with Recipient-derived Cells is Rare in Cardiac Allografts

Because recipient-derived circulating EPC could differentiate to EC in the transplanted heart, we determined the origin of cardiac allograft EC by using marker gene rats (Tie1/LacZ) or mice (GFP-BM) as allograft recipients.

When Tie1/LacZ allografts were transplanted to wild type (WT) recipients, areas with abundant X-gal reactivity in venous and arterial allograft endothelium was detected, indicating Tie1 expression in the donor EC.

Next, WT cardiac allografts were transplanted to Tie1/LacZ recipients to detect recipient-derived EC in the transplanted hearts. Only few donor-derived X-gal+EC, localizing to severely fibrotic areas, were seen in cross-sections in a total of 14 WT cardiac allografts.

Additionally, GFP-BM mice were used as cardiac allograft recipients, allowing the detection of BM-derived cells in the allografts. The majority of allograft-infiltrating CD11b+ myelomonocytic cells expressed GFP. Although GFP⁺ cells often surrounded allograft blood vessels, no co-localization with allograft CD31⁺ or c-kit+ capillaries was detected.

Donor- and recipient-specific MHC class I antibodies were used to identify the source of EC in allograft arteriosclerotic arteries. Numerous recipient MHC class I+ cells were found around occluded arteries, whereas only few positive cells were detected in the intima. In contrast, abundant donor MHC Class I immunoreactivity was found in the neointima. The contribution of recipient-derived SMC to neointimal formation was not assessed, as MHC Class I expression was low in SMC34.

VEGFR-2 Inhibition Normalizes C-kit+ and CD31⁺ Capillary Density in Chronically Rejecting Cardiac Allografts

To investigate the functional role of VEGFR-1 and -2, chronically rejecting mouse cardiac allograft recipients with suboptimal FK506 immunosuppression were treated with rat IgG (n=8); or with antibodies against VEGFR-1 (MF1, n=9), antibodies against VEGFR-2 (DC101, n=9), or both antibodies against VEGFR-1 and R-2 (n=10) for thirty days. Two months after heart transplantation, the antibodies targeting VEGFR-2 reduced the density of myocardial c-kit+ capillaries and CD31⁺ capillaries in the allograft to the level found in non-transplanted mouse hearts. VEGFR-1 inhibition also resulted in a smaller decrease in c-kit+ capillary density (p=NS with Dunnett's correction, p<0.05 with LSD correction). VEGFR-1 or -2 inhibition did not change the density of SMC coated vessels ((α-SMA+), indicating that VEGFR-2 inhibition specifically regulated angiogenesis at microvascular level.

VEGFR-1 and -2 Inhibition Reduces Inflammation in Chronically Rejecting Cardiac Allografts

Immunohistochemical analysis showed that targeting VEGFR-1, VEGFR-2, or both profoundly reduced the density of allograft-infiltrating CD11b+ myelomonocytic cells. VEGFR-2 inhibition also resulted in a similar reduction in CD8⁺ and CD4⁺ lymphocyte density in the allograft (for the combination group: p=NS with Dunnett's correction and p<0.05 with LSD correction).

VEGFR-1 and -2 Inhibition Reduces Arteriosclerosis in Chronically Rejecting Cardiac Allografts

Morphometrical analysis of allograft arteries revealed that targeting VEGFR-1, VEGFR-2, or both decreased the incidence of allograft arteries with intimal changes from about 55% in the IgG control mice to under 40% in the mice receiving anti-VEGFR-1 (p<0.05), anti-VEGFR-2 (p<0.05), or both (p<0.01). A similar result was also obtained on the mean occlusion of allograft arteries (about 18% arterial occlusion in the IgG controls, compared to about 7% in the anti-VEGFR-1 mice (p<0.01), about 11% in the anti-VEGFR-2 mice (p<0.05), and about 10% in the mice receiving both antibodies (p<0.05). These results indicate that both VEGFR-1 and -2 are involved in events leading to CAV.

Effect of VEGFR-1 and -2 Inhibition on Allograft Cytokine mRNA Levels

Finally, we used real time RT-PCR to determine the mRNA levels of inflammatory cytokines IFN-inducible protein-10 (IP-10) and monocyte chemotactic protein-1 (MCP-1) that are potentially regulated by VEGF in cardiac allografts, and the mRNA levels of stem cell factor (SCF) that is the ligand for c-kit. The analysis revealed that VEGFR-2 inhibition decreased allograft IP-10 mRNA by approximately 50% alone, and by 75% in combination with VEGFR-1 inhibition, and MCP-1 mRNA by 50%. In contrast, allograft TNF-α and SCF mRNA levels were similar in the control and treatment groups. These results indicate that the VEGFR-2 inhibition regulated at least in part the T cell and monocyte recruitment by decreasing IP-10 and MCP-1 production, respectively. Also, the effect of VEGFR-2-inhibition on c-kit+ capillaries was not associated with changes in SCF production.

### Analysis

Angiogenesis is a prominent feature in the intima and adventitia of cardiac allograft coronary arteries and it may be a driving force for the development of CAV. The results of these experiments demonstrate that, in addition to intimal and adventitial angiogenesis, chronic rejection induces the expression of primitive markers c-kit and VEGFR-2 in allograft myocardial capillaries. As the density of myocardial c-kit+ capillaries correlated with the severity of cardiac allograft inflammation and arteriosclerosis, alloimmune and ischemic stimuli may be important regulators of the myocardial angiogenesis we observed. This primitive ckit+ angiogenic response probably represents a repair process that, interestingly, in light of the present VEGF intervention results, may in fact aggravate inflammation and arteriosclerosis in transplanted hearts. Importantly, there may be a balance between early capillary formation and later destruction of allograft capillaries as seen in skin transplants. (See Moulton et al., “Angiogenesis in the huPBL-SCID model of human transplant rejection,” Transplantation, (1999); 67:1626-1631.)

In experiments using marker gene animals and donor- or recipient-specific antibodies, we found only few recipient-derived EC in the transplanted hearts and they were restricted to severely fibrotic areas. These observations suggest that recipient-derived circulating cells do not differentiate into allograft EC unless the injury to the allograft extensive. Although this notion argues against direct involvement of recipient-derived EPC in allograft angiogenesis, these circulating cells may have important paracrine effects. Our results on the origin and c-kit+ phenotype of allograft EC further indicates that donor-derived progenitor cells—such as resident cardiac stem cells or adventitial stem cells—directly participate in allograft angiogenesis. Alternatively, the hypoxic and inflammatory signals related to the transplantation may have induced dedifferentiation of allograft EC to a more primitive phenotype. Interestingly, EPC-derived soluble factors such as VEGF, VEGF-B, stromal cell derived factor-1, and insulin-like growth factor-1, and also hepatocyte growth factor may regulate the functions of c-kit+ cardiac progenitor cells, and the present results suggest important role for VEGFR-2.

VEGF is perhaps the most important angiogenic cytokine and it also has many proinflammatory properties. The present findings support the ory of regulatory role of VEGF in the pathogenesis of alloimmune responses and CAV in transplanted hearts, and shed light to the mechanisms, and the two VEGFR involved. In transplanted hearts VEGFR-2 inhibition reduced myocardial angiogenesis to the level seen in normal hearts, consistent with the important angiogenic role for VEGFR-2. In addition, targeting VEGFR-2 decreased inflammatory cell infiltration, and production of IP-10 and MCP-1 in the allograft, similarly to previous reports with anti-VEGF therapies. (See, e.g., Reinders et al., “Proinflammatory functions of vascular endothelial growth factor in alloimmunity,” J. Clin. Invest., (2003); 112:1655-1665.) Our results thus suggest that VEGFR-2 in cardiac allografts functions mainly at the endothelial level and regulates both pathological capillary angiogenesis and inflammation. Involvement of VEGFR-2 in cardiac inflammation may be a more general phenomenon, as the receptor participates in cardiac dysfunction during sepsis, and also in vascular permeability following myocardial infarction.

In contrast to VEGFR-2, VEGFR-1 was primarily found in allograft SMC and in peripheral blood myelomonocytic cells. As VEGFR-1 directly regulates SMC during arterial injury, VEGFR-1 inhibition in the current study may have directly decreased SMC recruitment to the intima. VEGFR-1 inhibition also profoundly reduced myelomonocyte recruitment to the allograft, consistent with its role in monocytes and inflammatory diseases. Although VEGFR-2 inhibition prominently decreased the density of myocardial c-kit+ cells, VEGFR-1 inhibition had a similar but more subtle effect. This indicates that also VEGFR-1 may in part regulate the capillary angiogenesis, and possibly involves cross-talk with VEGFR-2, or in-direct inflammation-mediated effects. The reason why combined VEGFR-1 and -2 inhibition did not have a beneficial additive effect may be explained by the moderate injury in the current experimental setting. Supporting this, our unpublished trachea transplantation findings show additive beneficial effect after severe but not after moderate tracheal injury. (See also Sho et al., “Function of the Vascular Endothelial Growth Factor Receptors Flt-1 and Flk-1/KDR in the Alloimmune Response In Vivo,” Transplantation, (2005); 80: 717-722.)

In summary, these experiments demonstrated that chronic rejection in cardiac allografts induced donor-derived capillary angiogenesis. Also, selective VEGFR-inhibition prevented allograft angiogenesis and had beneficial effects on inflammation and arteriosclerosis. These results indicate therapeutic applications for anti-VEGF strategies during pathological angiogenesis and inflammation in transplanted hearts.

### Example 9

### Combination Therapy Targeting Receptors of Multiple Growth Factors

The data in Examples 7 and 8 implicate VEGFR-1, VEGFR-2, and VEGFR-3 (and the growth factor ligands of these receptors) in chronic allograft rejection, particularly with reference to the model system used: cardiac transplants.

The experiments of Examples 7 and 8 are modified in that new combinations of inhibitors of growth factors and/or growth factor receptors are employed, and the protective effects of the combinations are evaluated. Evidence exists that the PDGF receptors and PDGF ligands may have a role in allograft disease. See, e.g., Nykanen et al., “Angiogenic Growth Factors in Cardiac Allograft Rejection,” Transplantation, (2006); 82: S22-S24, incorporated herein by reference. It is expected that combinations of inhibitors that are directed to receptors of distinct growth factor ligands (and/or directed to distinct growth factors themselves) will have additive or synergistic effects. All combinations described herein are specifically contemplated, including but not limited to the following:
(a) inhibitor of VEGFR-3 interaction with its ligands (VEGF-C or D), in combination with one or more inhibitors of VEGFR-1 and its ligands, or inhibitors of VEGFR-2 and its ligands, or both;
(b) inhibitor of VEGFR-3 interaction with its ligands, in combination with one or more inhibitors of PDGFR-alpha and its ligands, or PDGFR-beta and its ligands, or both;
(c) inhibitor of VEGFR-3 interaction with its ligands in combination with both (i) inhibitor of VEGFR-1 and its ligands, or inhibitor of VEGFR-2 and its ligands; and (ii) inhibitor of PDGFR-alpha and its ligands, or PDGFR-beta and its ligands;
(d) inhibitors of VEGFR-3, VEGFR-2, VEGFR-1, PDGFR-alpha, and PDGFR-beta with their respective ligands;
(e) inhibitors of VEGFR-1 or VEGFR-2 in combination with inhibitors of PDGFR-alpha or PDGFR-beta, and their respective ligands.

The inhibition of multiple receptors or ligands can be achieved with multivalent inhibitor substances described herein; small molecule non-specific inhibitors (e.g., tyrosine kinase inhibitors); or with co-administration of multiple, selective inhibitors, such as those described herein. The inhibition can be directed to inhibit ligand/receptor interaction; or to inhibit expression of the ligands or receptors; or to inhibit downstream signaling, for example. A composition comprising an inhibitor can be administered, or a composition comprising a pro-drug that is metabolized into an inhibitor can be administered; or a polynucleotide that encodes an inhibitor can be administered in a manner that achieves expression of the encoded inhibitor in the recipient organism.

### Example 10

### Inhibition of Rejection of Other Organ Grafts and in Other Species

The experiments described in Examples 7-9 are repeated in rodent models for all other organ transplants, including kidney, liver, lung, pancreas, intestine, and esophagus; to demonstrate that therapy directed to these molecular targest for intervention is effective with respect to recipients of other organ transplants.

The experiments described in Examples 7-9, or in the preceding paragraph, are repeated in larger mammals (e.g., felines, canines, porcines, equines, bovines, primates) to demonstrate efficacy in other species that may be considered more representative of humans, as a prerequisite to proving efficacy in human clinical trials.

The foregoing description and examples have been set forth merely to illustrate the invention and are not intended to be limiting. Because modifications of the disclosed embodiments incorporating the spirit and substance of the invention may occur to persons skilled in the art, the invention should be construed to include everything within the scope of the appended claims and equivalents thereof. The patents, patent application publications and other publications (e.g., Journal articles, and web/Internet materials) referenced herein are incorporated in their entirety.

Although the applicant(s) invented the full scope of the claims appended hereto, the claims are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

The patents, patent application publications and other publications (e.g., Journal articles) referenced herein are incorporated in their entirety.

What is claimed is:

1. A method for inducing tolerance or inhibiting rejection of a cell, tissue, or organ transplant, or for inhibiting arteriosclerosis in a transplant recipient, comprising: administering to a mammalian transplant recipient a composition that comprises an endothelial growth factor inhibitor, in an amount effective to induce tolerance for the transplant by the recipient, or inhibit rejection, or inhibit arteriosclerosis.

2. A method for inducing tolerance or inhibiting rejection of a cell, tissue, or organ transplant, or for inhibiting arteriosclerosis in a transplant recipient, comprising: administering to a mammalian transplant recipient a composition that comprises an nucleic acid that comprises a nucleotide sequence that encodes an endothelial growth factor inhibitor, wherein the nucleic acid is expressible in cells of the recipient or expressible in the transplanted cell, tissue, or organ to produce an amount of the endothelial growth factor inhibitor effective to induce tolerance for the transplant by the recipient, or inhibit rejection, or inhibit arteriosclerosis.

3. The method of claim 2, wherein the nucleic acid further comprises at least one expression control sequence operatively connected to the sequence that encodes the endothelial growth factor inhibitor.

4. The method of claim 2 or 3, comprising administering an expression vector that comprises the nucleic acid.

5. The method of claim 4, wherein the vector comprises a replication deficient viral vector.

6. The method of claim 5, wherein the vector comprises at least one member selected from the group consisting of a retrovirus, an adenovirus, an adeno-associated virus, a vaccinia virus and a herpesvirus.

7. The method of any one of claims 4-6, wherein expression of the vector is inducible by administration of an exogenous pharmaceutical agent.

8. The method of any one of claims 4-6, wherein expression of the vector is induced by an endogenous stress in the organ transplant recipient.

9. The method of claim 8, wherein the stress comprises an elevation of a biological marker correlated with rejection.

10. The method of any one of claims 1-9, wherein the recipient is human.

11. The method of any one of claim 10, wherein the transplant is a xenograft, and the method induces tolerance for the xenograft or inhibits xenograft rejection.

12. The method of any one of claims 1-10, wherein the transplant is an allograft transplant, and the composition is administered in an amount effective to induce tolerance for the allograft or inhibit alloimmunity.

13. The method of any one of claims 1-12, wherein the composition further comprises a pharmaceutically acceptable carrier.

14. The method of any one of claims 1-13, where the transplant is a cell or tissue transplant.

15. The method of claim 14, wherein the cell or tissue comprises a member selected from the group consisting of embryonic stem cells, pluripotent stem cells, hematopoietic precursor cells, neuronal precursor cells, and endothelial precursor cells.

16. The method of claim 14, wherein the cell or tissue comprises a member selected from the group consisting of pancreatic islet cells, cardiac myocytes, bone marrow cells, endothelial cells, and skin cells.

17. The method of any one of claims 1-13, wherein the transplant is an organ or organ fragment capable of performing functions of the organ or capable of regenerating into the organ.

18. The method of any one of claims 1-13, wherein the recipient received at least one transplanted organ, or fragment thereof, selected from the group consisting of a heart, a kidney, a lung, a liver, an intestine, a pancreas, skin, and bone.

19. The method of any one of claims 1-13, wherein the recipient received at least one transplanted organ selected from the group consisting of heart, lung, liver, and kidney.

20. The method of any one of claims 1-13, wherein the recipient received a heart transplant.

21. The method of any one of claims 1-20, wherein the composition is administered locally to the transplanted cell, tissue, or organ in the recipient.

22. The method of any one of claims 1-21, wherein the composition is administered systemically to the recipient.

23. The method of any one of claims 1-22, wherein the composition is administered intravenously, intramuscularly, or intraperitoneally.

24. The method of any one of claims 1-23, wherein the composition is administered perorally.

25. The method of any one of claims 1-24, further comprising administering the composition to the organ or the organ donor before the transplant.

26. The method of any one of claims 1-25, further comprising repeated administration of the composition to the recipient.

27. The method of any one of claims 1-26, wherein the composition is administered to the recipient perioperatively, relative to the transplant operation.

28. The method of any one of claims 1-27, wherein the composition is administered to the recipient for 1-90 days post-operatively, relative to the transplant operation.

29. The method of any one of claims 1-27, wherein the composition is administered to the recipient for 1-60 days post-operatively, relative to the transplant operation.

30. The method of any one of claims 1-27, wherein the composition is administered to the recipient for 1-30 days post-operatively, relative to the transplant operation.

31. The method of any one of claims 1-27, wherein the composition is administered to the recipient for 1-15 days post-operatively, relative to the transplant operation.

32. The method of any one of claims 1-31, comprising:
screening the organ transplant recipient for symptoms of an acute rejection reaction; and
administering the composition to the recipient upon detection of symptoms of acute rejection, in an amount effective to inhibit the rejection.

33. The method of any one of claims 1-32, wherein the endothelial growth factor inhibitor comprises a compound that inhibits stimulation of at least one receptor selected from the group consisting of VEGFR-1, VEGFR-2, and VEGFR-3, by a growth factor ligand of said at least one receptor.

34. The method of any one of claims 1-32, wherein the endothelial growth factor inhibitor comprises a compound that inhibits stimulation of VEGFR-3 by VEGF-C or inhibits stimulation of VEGFR-3 by VEGF-D.

35. The method of claim 34, wherein the compound comprises an antibody substance selected from the group consisting of antibody substances that immunoreacts with VEGFR-3, antibody substances that immunoreact with VEGF-C, and antibody substances that immunoreact with VEGF-D.

36. The method of claim 35, wherein the antibody substance is selected from the group consisting of a humanized antibody, a human antibody, a monoclonal antibody, a fragment of an antibody, and a polypeptide that comprises an antigen binding fragment of an antibody.

37. The method of claim 35, wherein the antibody substance is a monoclonal antibody that binds VEGFR-3 or VEGF-C or VEGF-D and inhibits binding between VEGFR-3 and VEGF-C or -D.

38. The method of any one of claims 1-34, wherein the endothelial growth factor inhibitor comprises a soluble receptor that binds to at least one endothelial cell growth factor.

39. The method of claim 38, wherein the endothelial growth factor inhibitor comprises a soluble VEGFR-3 polypeptide that binds to VEGF-C or VEGF-D.

40. The method of claim 39, wherein the soluble VEGFR-3 polypeptide comprises the VEGFR-3 extracellular domain, or a fragment thereof sufficient to bind VEGF-C or VEGF-D.

41. The method of claim 40, wherein the soluble VEGFR-3 polypeptide comprises the first and second immunoglobulin-like domains of the VEGFR-3.

42. The method of claim 40, wherein the soluble VEGFR-3 polypeptide comprises the first, second, and third immunoglobulin-like domains of the VEGFR-3.

43. The method of any one of claims 38-42, wherein the soluble receptor is fused to an immunoglobulin constant domain.

44. The method of any one of claims 1-34, wherein the inhibitor comprises a polypeptide that comprises an amino acid sequence at least 95% identical to amino acids 138-226 of SEQ ID NO: 6.

45. The method of any one of claims 1-34, wherein the inhibitor comprises a polypeptide that comprises an amino acid sequence at least 95% identical to amino acids 47-224 of SEQ ID NO: 6.

46. The method of any one of claims 1-34, wherein the inhibitor comprises a polypeptide that comprises an amino acid sequence at least 95% identical to amino acids 47-314 of SEQ ID NO: 6.

47. The method of any one of claims 1-34, wherein the inhibitor comprises a polypeptide that comprises an amino acid sequence at least 95% identical to amino acids 24-775 of SEQ ID NO: 6 or fragments thereof that bind VEGF-C.

48. The method of any one of claims 1-32, wherein the inhibitor comprises an antisense nucleic acid or an interfering RNA nucleic acid that inhibits expression of an endothelial cell growth factor or endothelial cell growth factor receptor.

49. The method of claim 48, wherein the inhibitor is a short interfering RNA that inhibits expression of a protein selected from the group consisting of VEGFR-3, VEGF-C, and VEGF-D.

50. The method of claim 48, wherein the inhibitor is an antisense nucleic acid that inhibits expression of a protein selected from the group consisting of VEGFR-3, VEGF-C, and VEGF-D.

51. The method of any one of claims 34-50, further comprising administering to the recipient a composition that comprises at least one growth factor inhibitor selected from the group consisting of:
inhibitors of VEGFR-1 with one or more of its ligands;
inhibitors of VEGFR-2 with one or more of its ligands;
inhibitors of PDFFR-alpha with one or more of its ligands;
inhibitors of PDGFR-beta with one or more of its ligands;
wherein the combination of inhibitors are administered in amounts effective to induce tolerance for the transplant by the recipient, or inhibit rejection, or inhibit arteriosclerosis.

52. The method of any one of claims 1-32, wherein the compound comprises bevacizumab (Avastin®) or Ranibizumab (Lucentis®).

53. The method of any one of claims 1-32, wherein the compound is a multivalent inhibitor of two or more receptors selected from the group consisting of VEGFR-1, VEGFR-2, and VEGFR-3.

54. The method of claim 53, wherein the multivalent compound inhibits VEGFR-3 and at least one receptor selected from VEGFR-1 and VEGFR-2.

55. The method of any one of claims 1-32, comprising administering to the recipient a composition that inhibits ligand binding to VEGFR-2 and inhibits ligand binding to VEGFR-3.

56. The method of any one of claims 1-55, further comprising administering an immunosuppressive agent to the organ transplant recipient.

57. The method of claim 56, wherein the immunosuppressive agent comprises at least one agent selected from the group consisting of corticosteriods, calcineurine inhibitors, antiproliferative agents, monoclonal antilymphocyte antibodies, and polyclonal antilymphocyte antibodies.

58. The method of claim 56, wherein the immunosuppressive agent comprises at least one compound selected from the group consisting of: Tacrolimus, Mycophenolic acid, Prednisone, Ciclosporin, Azathioprine, Basiliximab, Cyclosporine, Daclizumab, Muromonab-CD3, Mycophenolate Mofetil, Sirolimus, Methylprednisolone, Atgam, Thymoglobulin, OKT3, Rapamycin, Azathioprine, Cyclosporine, and Interleukin-2 Receptor Antagonist.

59. The method of any one of claims 1-58, further comprising administering an antibiotic or antifungal agent to the recipient.

60. The method of any one of claims 1-59, further comprising administering to a donor organism a composition that comprises an endothelial growth factor inhibitor, prior to harvesting a cell, tissue, or organ for transplantation into the recipient.

61. The method of any one of claims 1-59, further comprising contacting a cell, tissue, or organ with a composition that comprises an endothelial growth factor inhibitor, prior to transplanting the cell, tissue, or organ into the mammalian organ transplant recipient.

62. The method of any one of claims 1-59, further comprising administering to a donor organism, prior to harvesting cells, tissue, or an organ for transplantation, a composition that comprises an nucleic acid that comprises a nucleotide sequence that encodes an endothelial growth factor inhibitor, wherein the nucleic acid is expressible in cells of the tissue or organ to be transplanted.

63. The method of any one of claims 1-59, further comprising contacting a cell, tissue, or organ with a composition that comprises an nucleic acid that comprises a nucleotide sequence that encodes an endothelial growth factor inhibitor, prior to transplanting the cell, tissue, or organ into the recipient.

64. A method of preparing a donor cell, tissue, or organ for allograft or xenograft transplantation comprising contacting the cell, tissue, or organ with a composition that comprises an endothelial growth factor inhibitor, prior to transplanting the cell, tissue, or organ into a mammalian organ transplant recipient.

65. A method of preparing a donor cell, tissue, or organ for allograft or xenograft transplantation comprising contacting the cell, tissue, or organ with a composition that comprises an nucleic acid that comprises a nucleotide sequence that encodes an endothelial growth factor inhibitor, prior to transplanting the cell, tissue, or organ into a mammalian organ transplant recipient.

66. A composition that comprises an endothelial growth factor inhibitor, an immunosuppressant, and a pharmaceutically acceptable carrier.

67. A composition according to claim 66, wherein the inhibitor and the immunosuppressant are present in the composition in synergistically effective amounts.
